SEQUENCE LISTING (78 sequences)

SEQ ID NO: 1 - protein, 770 aa, homo sapiens
  Full STAT3 polypeptide sequence
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 2 - protein, 51 aa, homo sapiens
  STAT3 polypeptide encoded by Exon 8
SIVSELAGLLSAMEYVQKTLTDEELADWKRRQQIACIGGPPNICLDRLENW

SEQ ID NO: 3 - protein, 53 aa, homo sapiens
  STAT3 polypeptide encoded by Exon 9
ITSLAESQLQTRQQIKKLEELQQKVSYKGDPIVQHRPMLEERIVELFRNLMKS

SEQ ID NO: 4 - protein, 31 aa, homo sapiens
  STAT3 polypeptide encoded by Exon 10
AFVVERQPCMPMHPDRPLVIKTGVQFTTKVR

SEQ ID NO: 5 - protein, 20 aa, homo sapiens
  STAT3 polypeptide encoded by Exon 11
LLVKFPELNYQLKIKVCIDK

SEQ ID NO: 6 - protein, 10 aa, homo sapiens
  STAT3 polypeptide encoded by Exon 12
DSGDVAALRG

SEQ ID NO: 7 - protein, 31 aa, homo sapiens
  STAT3 polypeptide encoded by Exon 13
SRKFNILGTNTKVMNMEESNNGSLSAEFKHL

SEQ ID NO: 8 - protein, 16 aa, homo sapiens
  STAT3 polypeptide encoded by Exon 14
TLREQRCGNGGRANCD

SEQ ID NO: 9 - protein, 28 aa, homo sapiens
  STAT3 polypeptide encoded by Exon 15
ASLIVTEELHLITFETEVYHQGLKIDLE

SEQ ID NO: 10 - protein, 33 aa, homo sapiens
  STAT3 polypeptide encoded by Exon 16
THSLPVVVISNICQMPNAWASILWYNMLTNNPK

SEQ ID NO: 11 - protein, 45 aa, homo sapiens
  STAT3 polypeptide encoded by Exon 17
NVNFFTKPPIGTWDQVAEVLSWQFSSTTKRGLSIEQLTTLAEKLL

SEQ ID NO: 12 - protein, 17 aa, homo sapiens
  STAT3 polypeptide encoded by Exon 18
PGVNYSGCQITWAKFCK

SEQ ID NO: 13 - protein, 32 aa, homo sapiens
  STAT3 polypeptide encoded by Exon 19
ENMAGKGFSFWVWLDNIIDLVKKYILALWNEG

SEQ ID NO: 14 - protein, 47 aa, homo sapiens
  STAT3 polypeptide encoded by Exon 20
YIMGFISKERERAILSTKPPGTFLLRFSESSKEGGVTFTWVEKDISG

SEQ ID NO: 15 - protein, 71 aa, homo sapiens
  STAT3 polypeptide encoded by Exon 21
KTQIQSVEPYTKQQLNNMSFAEIIMGYKIMDATNILVSPLVYLYPDIPKEEAFGKYCRPESQEHPEADPGS

SEQ ID NO: 16 - protein, 14 aa, homo sapiens
  STAT3 polypeptide encoded by Exon 22
AAPYLKTKFICVTP

SEQ ID NO: 17 - protein, 38 aa, homo sapiens
  STAT3 polypeptide encoded by Exon 23
TTCSNTIDLPMSPRTLDSLMQFGNNGEGAEPSAGGQFE

SEQ ID NO: 18 - protein, 17 aa, homo sapiens
  STAT3 polypeptide encoded by Exon 24
SLTFDMELTSECATSPM

SEQ ID NO: 19 - protein, 770 aa, homo sapiens; identical to SEQ ID NO: 1
  STAT3alpha isoform

SEQ ID NO: 20 - protein, 721 aa, homo sapiens
  STAT3beta isoform
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGAAPYLKTKFICVTPFIDAVWK

SEQ ID NO: 21 - protein, 925 aa, artificial
  TALEN i7 Left monomer
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 22 - protein, 925 aa, artificial
  TALEN i7 right monomer
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 23 - protein, 925 aa, artificial
  TALEN i8b Left monomer
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 24 - protein, 925 aa, artificial
  TALEN i8b right monomer
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 25 - protein, 925 aa, artificial
  TALEN i9 Left monomer
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVHHGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 26 - protein, 925 aa, artificial
  TALEN i9 right monomer
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 27 - DNA, 280 bp, homo sapiens
  Intron 22 STAT3
gtaagtggcttcctttccccgttttgccttcatttctaatatcctcagttatccctgggaatgggacactgggtgagagttaatctgccaaaggttggaagcccctgggctatgtttagtactcaaagtgaccttgtgtgtttaaaaagcttgagcttttatttttctgttggagaccagagtttgatggcttgtgtgtgtgtgttttgttcttttttttttttccattgtgtcttgtcaaccccccgtttcccctcctgctgccccccatttcctacag

SEQ ID NO: 28 - DNA, 54 bp, artificial
  Artificial splice site
acaagctaggcccttttgctaatcatgttcatactgtcccttttttttccacag

SEQ ID NO: 29 - DNA, 54 bp, artificial
  Artificial splice site
acaagctaggcccttttgctaatcatgttcatactgtcccttttttttcctcag

SEQ ID NO: 30 - DNA, 176 bp, homo sapiens
  Intron 7 STAT3
gtaagggcataggtcggaccacttcccccatgtgtctcgctcacttgcgggatttcagcgtcttgtggcagaacttgcttggtttctaagaagttcctgctctggagttgactaaagaatgtggttagagacagtctgaggaaatgttttctgactttgttttggtttccaaccag

SEQ ID NO: 31 - DNA, 3385 bp, homo sapiens
  Intron 8 STAT3
gtaaaggatgaaagaagcttttcctttctttctcgaaagctagattgaattctgatcttaactgcaggcccacagaattggtactatatctccaacgtggggacttttccatattcaaatttagcccaagaattaaagtttttactttatttcggccaggcgctgtggctcacacctgtaatcccagcactttgggagaccaagatgggcggatcacttgaggtcaggagtttgagaccagcctggccaacatggtgaaaacacatctctactaaaaacataaaaaaattagccgggcgtggtggtgcgcacctgtagtcccagctactctgggcggctgaggcaggagaatcacttgaacctgggatatggaagttgcagtgagcggagatcttactaccgcacaccaaccagcctgggagacagagtgagactccatctcaaaaaaataaaaataaaataaagtttttactttatttggagaaactttgttttaaaaaatgtatttatattattatattttaagtatattttacttaataattcaattaaggcttttggtttaactgtatttaacagatagacaaaccttttaattttagttattttagtaatctaaaatgacacatgccctttttaagggaaaaaattcaaatacagaaaattaatcaagagaagaaaaaatttttaaatgaaatcatcagcagtactagtagttaaaatttagttgatgctcaatctagacatctgtcattatgtatatacacattatgtatatacacataaagatagaaatttatacagtttatattaggatcatttttttttctttttttggagtcagggtctcactgtgttacccagtctagagtacagttatgcagtcatggctcactggagccttgacctcctgggctcaggcagtcttcccaccttagccttctcagtagctgggactacaggcatgcaccaccacacctggctaatttttaaattttttatagagacagggtcttactttgttgcctgggctggtctcaaattcctgggctcaagggatcatcccacttcggcctctcaaaagctctggaattatagatgtgagctgccgtgcccagcccaggatcttcctttatatgcttttctgtaatttgcacttttaccttcatccagcatatcttactgcaacccttcctgtgcaaggccctatagtgagcatgttgcaccagcttgccttaggagaaacttgagatacagagcctgcactggaaatttagcgcaactctacatgagaatgcctgtctattcatatcctcactaaccctgagtgttgttaatttactgaaagcagttttaaatgcttcctgaccagggaacgaagaagcttaagttctgggaatgggaggatagaagtgccagaaaagagctcaggagttcagaaatccctgcagcggtccccctccctctcctttcactttctgtctttctggtcttttggtctttgttacactagtgataaaccatcaaagaatgatggaatgatgctaacttctctctttttttaatttttttgagacagagtctcactctgtcacccaggttagagtgcagtggcatgatcttggcttactgcaacctcctcctcccaggttcaagcgattcttagtcacaaccttccaagtagctgggattacaggcccatgccaccatgcctggctatttttttgtattttagtagatcgacctgcctcggcctctcaaatttttgggattacaggtgtcagccactgcacctggcctaatatctctattcttggagatagatttaatgagctttttctccctctctattcacttattccttgtgcatgttatcaatattttgaaacataatgtcatgtcctttgatcagttgaaggctgacattgaaaaggcttatggggattgggtgttgtggctcacgcctgtaaatcccaatgctttgggaggcagagtcgggaggatcacttgaacccaggagtttgagaccagcctgggcaacaaagtgagatcccatccctacaaaaatttaaaaaactagacatgtgccattacacttcagcctgggtgacagagtgagactccatctcaaaaaactaaactaaactaaacaggcatggtggcacacacctatagttctagctactcaggaagctgaggtaggaggatcactcatgtccaggagttggaggaggcagtgagctatgatcatgccattgcactgcactaggccacagagtgggaccctgtctcaaaaaaaaaaaaagaaagaaagaaaagaaagggctcatgtagttcaagcccttctcttcatgcaaggggatgctaaggcccatgatggtgaagggcctggcaaagcttgcacagatagtgtgtgacagagctggctcaaacccatctttgggagctgtctaatctctttttctgagtctttatgttcatagacaagttaggatgagtaaagtaagtgctaaattccatatttcgtgttctgcatatctgggctcagatgcttgtcattttccagtgataactccatcaatgcctcctagtggtataaattttaatacttcttgtgtgcccagccccctcttagaaatttgagattttaggaagggactagtaataaaaggtaaaataaattattttctggccaggcatggtggctcacacctgtaatgccagtacttcgggaggtcgaggcagatggatcacctgaggtcaggagttcaagaccagcctggccaacaaggcaaaatcccatctctactaaaaatgcaaaaattatccgggagtggtggtgggtgcctgtaatcccagctacttgggaggctgaggcaggagaatcacttgaacttgggaggcggaggttgcagtgagctgagactgtgccactgcactccagcctgggcaacagagtaagactctatctcaaaaaaaaaaaaaaaaaaaaaaaggccaggcgcagtggcttacacctgtaatctctcaggaggctcaggcaggagaatcacttgaacccgggaaatggaggttgcagtgagccgagattgcaccactgcactccagctcaaaaaataaataaataaataaattattttctttttttatttattttttcagcatccacccaacatggtgaaaaattcctcttttcttaatgtcactgaactgtaaacttaagatgaaaaattgtaaatttcatgctatatatatttcaccacaataaaaaaattccttgttcttattgtagtggtctccatgtcttcagtatttccttccccttctccatctcacctgtatacattcactttggtaattagcatctttcttaatttattggcag

SEQ ID NO: 32 - DNA, 125 bp, homo sapiens
  Intron 9 STAT3
gtaatttagcatccttgtccctttccctcatctaaaaaatacctaaagactcacgtggtagagtgagaggcgggctgacttctggtcatggccgtggcgcgtgagcccatcttctctttcctcag

SEQ ID NO: 33 - DNA, 2253 bp, artificial
  STAT3 inserted template sequence 1
tgatacaagctaggcccttttgctaatcatgttcatactgtcccttttttttccacagagcatcgtgagtgagctggctgggttgctttcagctatggaatatgtgcaaaagacgttgacagacgaggaactggccgactggaagcggaggcaacaaattgcttgcattggaggaccacccaatatatgcctggatagattggagaactggataacgtcattagcagaaagccaactgcaaactagacagcaaataaagaagctggaagaactccagcaaaaagtttcctataagggtgaccctatagtgcagcacaggcccatgttggaggaacgcattgtggagttgtttagaaatctgatgaaaagtgcctttgtggtggagcgacaaccgtgcatgcctatgcaccctgaccggcccttggtgataaagaccggagtacaattcactactaaagtgaggctgctcgtaaagttccctgaactcaattaccagttgaagataaaagtgtgcatcgataaggactccggcgatgttgccgcgctccggggaagtcgaaagttcaacatcttgggcaccaatacaaaggtgatgaacatggaggaatcaaacaacgggtccctttccgcggagttcaaacatttgacattgcgcgagcagagatgtgggaacgggggaagagcaaattgtgacgcttctctgatcgtaactgaggagctgcatctcataacattcgagactgaggtctaccatcagggattgaagatcgacctcgaaactcattcactgcctgtagttgttattagtaatatttgccagatgccgaatgcgtgggctagcatactttggtataacatgcttacgaataatccaaagaacgttaattttttcactaaacctccaatcggaacttgggaccaggtcgcagaagtgctgagctggcaattttcctccaccaccaaacgcggtcttagcatcgaacaattgacgaccctcgcagaaaagcttttgggtcctggcgttaattactcagggtgtcagataacttgggcaaagttctgcaaagaaaacatggcaggtaagggcttttcattttgggtctggctggataacattatagatttggtgaaaaagtacattcttgccctgtggaacgaaggctacatcatgggtttcattagcaaggagcgcgagagagcgatcctgtcaactaaaccccctggaacgtttctccttagattttccgaaagcagcaaggaaggtggcgtgactttcacgtgggttgaaaaggacatcagcggaaagacgcaaattcagagcgttgaaccttacaccaagcagcaacttaacaacatgagcttcgccgagataataatgggttacaaaataatggacgctactaatatcctcgtctcacccctcgtgtacttgtatccagacattcccaaagaggaggcgttcgggaagtactgtcgcccggagtcacaagaacatcctgaagctgacccaggaagtgcagctccgtaccttaagaccaagtttatctgtgtgacaccgtaagtggcttcctttccccgttttgccttcatttctaatatcctcagttatccctgggaatgggacactgggtgagagttaatctgccaaaggttggaagcccctgggctatgtttagtactcaaagtgaccttgtgtgtttaaaaagcttgagcttttatttttctgttggaagaccagagtttgatggcttgtgtgtgtgtgttttgttcttttttttttttccattgtgtcttgtcaaccccccgtttcccctcctgctgccccccatttcctacagaacgacctgcagcaataccattgacctgccgatgtccccccgcactttagattcattgatgcagtttggaaataatggtgaaggtgctgaaccatccgctggtggacagtttgagagccttacgtttgatatggagttgacaagcgagtgtgctacatcacctatgtgagcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacggcttgattatgatca

SEQ ID NO: 34 - DNA, 2252 bp, artificial
  STAT3 inserted template sequence 2
tgatacaagctaggcccttttgctaatcatgttcatactgtcccttttttttcctcagagcatcgtgagtgagctggctgggttgctttcagctatggaatatgtgcaaaagacgttgacagacgaggaactggccgactggaagcggaggcaacaaattgcttgcattggaggaccacccaatatatgcctggatagattggagaactggataacgtcattagcagaaagccaactgcaaactagacagcaaataaagaagctggaagaactccagcaaaaagtttcctataagggtgaccctatagtgcagcacaggcccatgttggaggaacgcattgtggagttgtttagaaatctgatgaaaagtgcctttgtggtggagcgacaaccgtgcatgcctatgcaccctgaccggcccttggtgataaagaccggagtacaattcactactaaagtgaggctgctcgtaaagttccctgaactcaattaccagttgaagataaaagtgtgcatcgataaggactccggcgatgttgccgcgctccggggaagtcgaaagttcaacatcttgggcaccaatacaaaggtgatgaacatggaggaatcaaacaacgggtccctttccgcggagttcaaacatttgacattgcgcgagcagagatgtgggaacgggggaagagcaaattgtgacgcttctctgatcgtaactgaggagctgcatctcataacattcgagactgaggtctaccatcagggattgaagatcgacctcgaaactcattcactgcctgtagttgttattagtaatatttgccagatgccgaatgcgtgggctagcatactttggtataacatgcttacgaataatccaaagaacgttaattttttcactaaacctccaatcggaacttgggaccaggtcgcagaagtgctgagctggcaattttcctccaccaccaaacgcggtcttagcatcgaacaattgacgaccctcgcagaaaagcttttgggtcctggcgttaattactcagggtgtcagataacttgggcaaagttctgcaaagaaaacatggcaggcaagggcttttcattttgggtctggctggataacattatagatttggtgaaaaagtacattcttgccctgtggaacgaaggctacatcatgggtttcattagcaaggagcgcgagagagcgatcctgtcaactaaaccccctggaacgtttctccttcgcttttccgaaagcagcaaggaaggtggcgtgactttcacgtgggttgaaaaggacatcagcggaaagacgcaaattcagagcgttgaaccttacaccaagcagcaacttaacaacatgagcttcgccgagataataatgggttacaaaataatggacgctactaatatcctcgtctcacccctcgtgtacttgtatccggacattcccaaagaggaggcgttcgggaagtactgtcgcccggagtcacaagaacatcctgaagctgacccaggaagtgcagctccgtaccttaagaccaagtttatctgcgtgacaccgtaagtggcttcctttccccgttttgccttcatttctaatatcctcagttatccctgggaatgggacactgggtgagagttaatctgccaaaggttggaagcccctgggctatgtttagtactcaaagtgaccttgtgtgtttaaaaagcttgagcttttatttttctgttggagaccagagtttgatggcttgtgtgtgtgtgttttgttcttttttttttttccattgtgtcttgtcaaccccccgtttcccctcctgctgccccccatttcctacagaacgacctgcagcaataccattgacctgccgatgtccccccgcactttagattcattgatgcagtttggaaataatggtgaaggtgctgaaccatccgctggtggacagtttgagagccttacgtttgatatggagttgacaagcgagtgtgctacatcacctatgtgagcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacggcttgattatgatca

SEQ ID NO: 35 - DNA, 2252 bp, artificial
  STAT3 inserted template sequence 3
tgatacaagctaggcccttttgctaatcatgttcatactgtcccttttttttcctcagagcatcgtgagtgagctggctgggttgctttcagctatggaatatgtgcaaaagacgttgacagacgaggaactggccgactggaagcggaggcaacaaattgcttgcattggaggaccacccaatatatgcctggatagattggagaactggataacgtcattagcagaaagccaactgcaaactagacagcaaataaagaagctggaagaactccagcaaaaagtttcctataagggtgaccctatagtgcagcacaggcccatgttggaggaacgcattgtggagttgtttagaaatctgatgaaaagtgcctttgtggtggagcgacaaccgtgcatgcctatgcaccctgaccggcccttggtgataaagaccggagtacaattcactactaaagtgaggctgctcgtaaagttccctgaactcaattaccagttgaagataaaagtgtgcatcgataaggactccggcgatgttgccgcgctccggggaagtcgaaagttcaacatcttgggcaccaatacaaaggtgatgaacatggaggaatcaaacaacgggtccctttccgcggagttcaaacatttgacattgcgcgagcagagatgtgggaacgggggaagagcaaattgtgacgcttctctgatcgtaactgaggagctgcatctcataacattcgagactgaggtctaccatcagggattgaagatcgacctcgaaactcattcactgcctgtagttgttattagtaatatttgccagatgccgaatgcgtgggctagcatactttggtataacatgcttacgaataatccaaagaacgttaattttttcactaaacctccaatcggaacttgggaccaggtcgcagaagtgctgagctggcaattttcctccaccaccaaacgcggtcttagcatcgaacaattgacgaccctcgcagaaaagcttttgggtcctggcgttaattactcagggtgtcagataacttgggcaaagttctgcaaagaaaacatggcaggcaagggcttttcattttgggtctggctggataacattatagatttggtgaaaaagtacattcttgccctgtggaacgaaggctacatcatgggtttcattagcaaggagcgcgagagagcgatcctgtcaactaaaccccctggaacgtttctccttcgcttttccgaaagcagcaaggaaggtggcgtgactttcacgtgggttgaaaaggacatcagcggaaagacgcaaattcagagcgttgaaccttacaccaagcagcaacttaacaacatgagcttcgccgagataataatgggttacaaaataatggacgctactaatatcctcgtctcacccctcgtgtacttgtatccggacattcccaaagaggaggcgttcgggaagtactgtcgcccggagtcacaagaacatcctgaagctgacccaggaagtgcagctccgtaccttaagaccaagtttatctgtgtgacaccgtaagtggcttcctttccccgttttgccttcatttctaatatcctcagttatccctgggaatgggacactgggtgagagttaatctgccaaaggttggaagcccctgggctatgtttagtactcaaagtgaccttgtgtgtttaaaaagcttgagcttttatttttctgttggagaccagagtttgatggcttgtgtgtgtgtgttttgttcttttttttttttccattgtgtcttgtcaaccccccgtttcccctcctgctgccccccatttcctacagaacgacctgcagcaataccattgacctgccgatgtccccccgcactttagattcattgatgcagtttggaaataatggtgaaggtgctgaaccatccgctggtggacagtttgagagccttacgtttgatatggagttgacaagcgagtgtgctacatcacctatgtgagcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacggcttgattatgatca

SEQ ID NO: 36 - DNA, 2252 bp, artificial
  STAT3 inserted template sequence 4
tgatacaagctaggcccttttgctaatcatgttcatactgtcccttttttttcctcagagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagcgcctttgtggtggagcggcagccctgcatgcccatgcatcctgaccggcccctcgtcatcaagaccggcgtccagttcactactaaagtcagattgctggtcaaattccctgagttgaattatcagcttaaaattaaagtgtgcattgacaaagactctggggacgttgcagctctcagaggatcccggaagttcaacatcttgggcaccaacacaaaagtgatgaacatggaagaatccaacaacggcagcctctctgccgaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgaacaattgacgaccctggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaaggatacatcatgggctttatcagtaaggagcgggagagagcgatcctgtcaactaaacctccaggcaccttcctgctaagattcagtgaaagcagcaaagaaggaggcgtcactttcacttgggtggagaaggacatcagcggaaagacgcaaattcagagcgtggaaccatacacaaagcagcagctgaacaacatgtcatttgctgaaatcatcatgggctataagatcatggatgctaccaatatcctggtgtctccactggtctatctctatccggacattcccaaagaggaggcgttcggaaagtactgtcgcccggagtcacaggagcatcctgaagctgacccaggcagcgctgccccatacctgaagaccaagtttatctgtgtgacaccgtaagtggcttcctttccccgttttgccttcatttctaatatcctcagttatccctgggaatgggacactgggtgagagttaatctgccaaaggttggaagcccctgggctatgtttagtactcaaagtgaccttgtgtgtttaaaaagcttgagcttttatttttctgttggagaccagagtttgatggcttgtgtgtgtgtgttttgttcttttttttttttccattgtgtcttgtcaaccccccgtttcccctcctgctgccccccatttcctacagaacgacctgcagcaataccattgacctgccgatgtccccccgcactttagattcattgatgcagtttggaaataatggtgaaggtgctgaaccctcagcaggaggacagtttgagagccttacgtttgatatggagttgacctcggagtgcgctacctcccccatgtgagcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacggcttgattatgatca

SEQ ID NO: 37 - DNA, 2252 bp, artificial
  STAT3 inserted template sequence 5
tgatacaagctaggcccttttgctaatcatgttcatactgtcccttttttttcctcagagcatcgtgagtgagctggctgggttgctttcagctatggaatatgtgcaaaagacgttgacagacgaggaactggccgactggaagcggaggcaacaaattgcttgcattggaggaccacccaatatatgcctggatagattggagaactggataacgtcattagcagaaagccaactgcaaactagacagcaaataaagaagctggaagaactccagcaaaaagtttcctataagggtgaccctatagtgcagcacaggcccatgttggaggaacgcattgtggagttgtttagaaatctgatgaaaagtgcctttgtggtggagcgacaaccgtgcatgcctatgcaccctgaccggcccttggtgataaagaccggagtacaattcactactaaagtgaggctgctcgtaaagttccctgaactcaattaccagttgaagataaaagtgtgcatcgataaagactctggggacgttgcagctctcagaggatcccggaagttcaacatcttgggcaccaacacaaaagtgatgaacatggaagaatccaacaacggcagcctctctgccgaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgaacaattgacgaccctggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaaggatacatcatgggctttatcagtaaggagcgggagagagcgatcctgtcaactaaacctccaggcaccttcctgctaagattcagtgaaagcagcaaagaaggaggcgtcactttcacttgggtggagaaggacatcagcggaaagacgcaaattcagagcgtggaaccatacacaaagcagcagctgaacaacatgtcatttgctgaaatcatcatgggctataagatcatggatgctaccaatatcctggtgtctccactggtctatctctatccggacattcccaaagaggaggcgttcggaaagtactgtcgcccggagtcacaggagcatcctgaagctgacccaggcagcgctgccccatacctgaagaccaagtttatctgtgtgacaccgtaagtggcttcctttccccgttttgccttcatttctaatatcctcagttatccctgggaatgggacactgggtgagagttaatctgccaaaggttggaagcccctgggctatgtttagtactcaaagtgaccttgtgtgtttaaaaagcttgagcttttatttttctgttggagaccagagtttgatggcttgtgtgtgtgtgttttgttcttttttttttttccattgtgtcttgtcaaccccccgtttcccctcctgctgccccccatttcctacagaacgacctgcagcaataccattgacctgccgatgtccccccgcactttagattcattgatgcagtttggaaataatggtgaaggtgctgaaccctcagcaggaggacagtttgagagccttacgtttgatatggagttgacctcggagtgcgctacctcccccatgtgagcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacggcttgattatgatca

SEQ ID NO: 38 - DNA, 49 bp, homo sapiens
  target sequence TALEN i7
tctaagaagttcctgctctggagttgactaaagaatgtggttagagaca

SEQ ID NO: 39 - DNA, 49 bp, homo sapiens
  target sequence TALEN i8b
tagaaatttgagattttaggaagggactagtaataaaaggtaaaataaa

SEQ ID NO: 40 - DNA, 49 bp, homo sapiens
  target sequence TALEN i9
tgacttctggtcatggccgtggcgcgtgagcccatcttctctttcctca

SEQ ID NO: 41 - DNA, 2922 bp, artificial
  STAT3  DNA repair template intron 7 sequence 1
gtcaaggagtattccctcaggtcaaggagttttttcttccttcgcagacatgcaagatctgaatggaaacaaccagtcagtgaccaggcagaagatgcagcagctggaacagatgctcactgcgctggaccagatgcggagagtaagggcataggtcggaccacttcccccatgtgtctcgctcacttgcgggatttcagcgtcttgtggcagaacttgcttggtttctaagaagttcctgctctggagttgatacaagctaggcccttttgctaatcatgttcatactgtcccttttttttccacagagcatcgtgagtgagctggctgggttgctttcagctatggaatatgtgcaaaagacgttgacagacgaggaactggccgactggaagcggaggcaacaaattgcttgcattggaggaccacccaatatatgcctggatagattggagaactggataacgtcattagcagaaagccaactgcaaactagacagcaaataaagaagctggaagaactccagcaaaaagtttcctataagggtgaccctatagtgcagcacaggcccatgttggaggaacgcattgtggagttgtttagaaatctgatgaaaagtgcctttgtggtggagcgacaaccgtgcatgcctatgcaccctgaccggcccttggtgataaagaccggagtacaattcactactaaagtgaggctgctcgtaaagttccctgaactcaattaccagttgaagataaaagtgtgcatcgataaggactccggcgatgttgccgcgctccggggaagtcgaaagttcaacatcttgggcaccaatacaaaggtgatgaacatggaggaatcaaacaacgggtccctttccgcggagttcaaacatttgacattgcgcgagcagagatgtgggaacgggggaagagcaaattgtgacgcttctctgatcgtaactgaggagctgcatctcataacattcgagactgaggtctaccatcagggattgaagatcgacctcgaaactcattcactgcctgtagttgttattagtaatatttgccagatgccgaatgcgtgggctagcatactttggtataacatgcttacgaataatccaaagaacgttaattttttcactaaacctccaatcggaacttgggaccaggtcgcagaagtgctgagctggcaattttcctccaccaccaaacgcggtcttagcatcgaacaattgacgaccctcgcagaaaagcttttgggtcctggcgttaattactcagggtgtcagataacttgggcaaagttctgcaaagaaaacatggcaggtaagggcttttcattttgggtctggctggataacattatagatttggtgaaaaagtacattcttgccctgtggaacgaaggctacatcatgggtttcattagcaaggagcgcgagagagcgatcctgtcaactaaaccccctggaacgtttctccttagattttccgaaagcagcaaggaaggtggcgtgactttcacgtgggttgaaaaggacatcagcggaaagacgcaaattcagagcgttgaaccttacaccaagcagcaacttaacaacatgagcttcgccgagataataatgggttacaaaataatggacgctactaatatcctcgtctcacccctcgtgtacttgtatccagacattcccaaagaggaggcgttcgggaagtactgtcgcccggagtcacaagaacatcctgaagctgacccaggaagtgcagctccgtaccttaagaccaagtttatctgtgtgacaccgtaagtggcttcctttccccgttttgccttcatttctaatatcctcagttatccctgggaatgggacactgggtgagagttaatctgccaaaggttggaagcccctgggctatgtttagtactcaaagtgaccttgtgtgtttaaaaagcttgagcttttatttttctgttggaagaccagagtttgatggcttgtgtgtgtgtgttttgttcttttttttttttccattgtgtcttgtcaaccccccgtttcccctcctgctgccccccatttcctacagaacgacctgcagcaataccattgacctgccgatgtccccccgcactttagattcattgatgcagtttggaaataatggtgaaggtgctgaaccatccgctggtggacagtttgagagccttacgtttgatatggagttgacaagcgagtgtgctacatcacctatgtgagcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacggcttgattatgatcatgactaaagaatgtggttagagacagtctgaggaaatgttttctgactttgttttggtttccaaccagagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggtaaaggatgaaagaagcttttcctttctttctcgaaagctagattgaattctgatcttaactgcaggcccacagaattggtactatatctccaacgtggggacttttccatattcaaatttagcccaagaattaaagtttttactttatttcggccaggcgctgtggctcacacctgtaatcccagcactttgggaga

SEQ ID NO: 42 - DNA, 2921 bp, artificial
  STAT3  DNA repair template intron 7 sequence 2
gtcaaggagtattccctcaggtcaaggagttttttcttccttcgcagacatgcaagatctgaatggaaacaaccagtcagtgaccaggcagaagatgcagcagctggaacagatgctcactgcgctggaccagatgcggagagtaagggcataggtcggaccacttcccccatgtgtctcgctcacttgcgggatttcagcgtcttgtggcagaacttgcttggtttctaagaagttcctgctctggagttgatacaagctaggcccttttgctaatcatgttcatactgtcccttttttttcctcagagcatcgtgagtgagctggctgggttgctttcagctatggaatatgtgcaaaagacgttgacagacgaggaactggccgactggaagcggaggcaacaaattgcttgcattggaggaccacccaatatatgcctggatagattggagaactggataacgtcattagcagaaagccaactgcaaactagacagcaaataaagaagctggaagaactccagcaaaaagtttcctataagggtgaccctatagtgcagcacaggcccatgttggaggaacgcattgtggagttgtttagaaatctgatgaaaagtgcctttgtggtggagcgacaaccgtgcatgcctatgcaccctgaccggcccttggtgataaagaccggagtacaattcactactaaagtgaggctgctcgtaaagttccctgaactcaattaccagttgaagataaaagtgtgcatcgataaggactccggcgatgttgccgcgctccggggaagtcgaaagttcaacatcttgggcaccaatacaaaggtgatgaacatggaggaatcaaacaacgggtccctttccgcggagttcaaacatttgacattgcgcgagcagagatgtgggaacgggggaagagcaaattgtgacgcttctctgatcgtaactgaggagctgcatctcataacattcgagactgaggtctaccatcagggattgaagatcgacctcgaaactcattcactgcctgtagttgttattagtaatatttgccagatgccgaatgcgtgggctagcatactttggtataacatgcttacgaataatccaaagaacgttaattttttcactaaacctccaatcggaacttgggaccaggtcgcagaagtgctgagctggcaattttcctccaccaccaaacgcggtcttagcatcgaacaattgacgaccctcgcagaaaagcttttgggtcctggcgttaattactcagggtgtcagataacttgggcaaagttctgcaaagaaaacatggcaggcaagggcttttcattttgggtctggctggataacattatagatttggtgaaaaagtacattcttgccctgtggaacgaaggctacatcatgggtttcattagcaaggagcgcgagagagcgatcctgtcaactaaaccccctggaacgtttctccttcgcttttccgaaagcagcaaggaaggtggcgtgactttcacgtgggttgaaaaggacatcagcggaaagacgcaaattcagagcgttgaaccttacaccaagcagcaacttaacaacatgagcttcgccgagataataatgggttacaaaataatggacgctactaatatcctcgtctcacccctcgtgtacttgtatccggacattcccaaagaggaggcgttcgggaagtactgtcgcccggagtcacaagaacatcctgaagctgacccaggaagtgcagctccgtaccttaagaccaagtttatctgcgtgacaccgtaagtggcttcctttccccgttttgccttcatttctaatatcctcagttatccctgggaatgggacactgggtgagagttaatctgccaaaggttggaagcccctgggctatgtttagtactcaaagtgaccttgtgtgtttaaaaagcttgagcttttatttttctgttggagaccagagtttgatggcttgtgtgtgtgtgttttgttcttttttttttttccattgtgtcttgtcaaccccccgtttcccctcctgctgccccccatttcctacagaacgacctgcagcaataccattgacctgccgatgtccccccgcactttagattcattgatgcagtttggaaataatggtgaaggtgctgaaccatccgctggtggacagtttgagagccttacgtttgatatggagttgacaagcgagtgtgctacatcacctatgtgagcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacggcttgattatgatcatgactaaagaatgtggttagagacagtctgaggaaatgttttctgactttgttttggtttccaaccagagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggtaaaggatgaaagaagcttttcctttctttctcgaaagctagattgaattctgatcttaactgcaggcccacagaattggtactatatctccaacgtggggacttttccatattcaaatttagcccaagaattaaagtttttactttatttcggccaggcgctgtggctcacacctgtaatcccagcactttgggaga

SEQ ID NO: 43 - DNA, 2921 bp, artificial
  STAT3  DNA repair template intron 7 sequence 3
gtcaaggagtattccctcaggtcaaggagttttttcttccttcgcagacatgcaagatctgaatggaaacaaccagtcagtgaccaggcagaagatgcagcagctggaacagatgctcactgcgctggaccagatgcggagagtaagggcataggtcggaccacttcccccatgtgtctcgctcacttgcgggatttcagcgtcttgtggcagaacttgcttggtttctaagaagttcctgctctggagttgatacaagctaggcccttttgctaatcatgttcatactgtcccttttttttcctcagagcatcgtgagtgagctggctgggttgctttcagctatggaatatgtgcaaaagacgttgacagacgaggaactggccgactggaagcggaggcaacaaattgcttgcattggaggaccacccaatatatgcctggatagattggagaactggataacgtcattagcagaaagccaactgcaaactagacagcaaataaagaagctggaagaactccagcaaaaagtttcctataagggtgaccctatagtgcagcacaggcccatgttggaggaacgcattgtggagttgtttagaaatctgatgaaaagtgcctttgtggtggagcgacaaccgtgcatgcctatgcaccctgaccggcccttggtgataaagaccggagtacaattcactactaaagtgaggctgctcgtaaagttccctgaactcaattaccagttgaagataaaagtgtgcatcgataaggactccggcgatgttgccgcgctccggggaagtcgaaagttcaacatcttgggcaccaatacaaaggtgatgaacatggaggaatcaaacaacgggtccctttccgcggagttcaaacatttgacattgcgcgagcagagatgtgggaacgggggaagagcaaattgtgacgcttctctgatcgtaactgaggagctgcatctcataacattcgagactgaggtctaccatcagggattgaagatcgacctcgaaactcattcactgcctgtagttgttattagtaatatttgccagatgccgaatgcgtgggctagcatactttggtataacatgcttacgaataatccaaagaacgttaattttttcactaaacctccaatcggaacttgggaccaggtcgcagaagtgctgagctggcaattttcctccaccaccaaacgcggtcttagcatcgaacaattgacgaccctcgcagaaaagcttttgggtcctggcgttaattactcagggtgtcagataacttgggcaaagttctgcaaagaaaacatggcaggcaagggcttttcattttgggtctggctggataacattatagatttggtgaaaaagtacattcttgccctgtggaacgaaggctacatcatgggtttcattagcaaggagcgcgagagagcgatcctgtcaactaaaccccctggaacgtttctccttcgcttttccgaaagcagcaaggaaggtggcgtgactttcacgtgggttgaaaaggacatcagcggaaagacgcaaattcagagcgttgaaccttacaccaagcagcaacttaacaacatgagcttcgccgagataataatgggttacaaaataatggacgctactaatatcctcgtctcacccctcgtgtacttgtatccggacattcccaaagaggaggcgttcgggaagtactgtcgcccggagtcacaagaacatcctgaagctgacccaggaagtgcagctccgtaccttaagaccaagtttatctgtgtgacaccgtaagtggcttcctttccccgttttgccttcatttctaatatcctcagttatccctgggaatgggacactgggtgagagttaatctgccaaaggttggaagcccctgggctatgtttagtactcaaagtgaccttgtgtgtttaaaaagcttgagcttttatttttctgttggagaccagagtttgatggcttgtgtgtgtgtgttttgttcttttttttttttccattgtgtcttgtcaaccccccgtttcccctcctgctgccccccatttcctacagaacgacctgcagcaataccattgacctgccgatgtccccccgcactttagattcattgatgcagtttggaaataatggtgaaggtgctgaaccatccgctggtggacagtttgagagccttacgtttgatatggagttgacaagcgagtgtgctacatcacctatgtgagcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacggcttgattatgatcatgactaaagaatgtggttagagacagtctgaggaaatgttttctgactttgttttggtttccaaccagagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggtaaaggatgaaagaagcttttcctttctttctcgaaagctagattgaattctgatcttaactgcaggcccacagaattggtactatatctccaacgtggggacttttccatattcaaatttagcccaagaattaaagtttttactttatttcggccaggcgctgtggctcacacctgtaatcccagcactttgggaga

SEQ ID NO: 44 - DNA, 2921 bp, artificial
  STAT3  DNA repair template intron 7 sequence 4
gtcaaggagtattccctcaggtcaaggagttttttcttccttcgcagacatgcaagatctgaatggaaacaaccagtcagtgaccaggcagaagatgcagcagctggaacagatgctcactgcgctggaccagatgcggagagtaagggcataggtcggaccacttcccccatgtgtctcgctcacttgcgggatttcagcgtcttgtggcagaacttgcttggtttctaagaagttcctgctctggagttgatacaagctaggcccttttgctaatcatgttcatactgtcccttttttttcctcagagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagcgcctttgtggtggagcggcagccctgcatgcccatgcatcctgaccggcccctcgtcatcaagaccggcgtccagttcactactaaagtcagattgctggtcaaattccctgagttgaattatcagcttaaaattaaagtgtgcattgacaaagactctggggacgttgcagctctcagaggatcccggaagttcaacatcttgggcaccaacacaaaagtgatgaacatggaagaatccaacaacggcagcctctctgccgaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgaacaattgacgaccctggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaaggatacatcatgggctttatcagtaaggagcgggagagagcgatcctgtcaactaaacctccaggcaccttcctgctaagattcagtgaaagcagcaaagaaggaggcgtcactttcacttgggtggagaaggacatcagcggaaagacgcaaattcagagcgtggaaccatacacaaagcagcagctgaacaacatgtcatttgctgaaatcatcatgggctataagatcatggatgctaccaatatcctggtgtctccactggtctatctctatccggacattcccaaagaggaggcgttcggaaagtactgtcgcccggagtcacaggagcatcctgaagctgacccaggcagcgctgccccatacctgaagaccaagtttatctgtgtgacaccgtaagtggcttcctttccccgttttgccttcatttctaatatcctcagttatccctgggaatgggacactgggtgagagttaatctgccaaaggttggaagcccctgggctatgtttagtactcaaagtgaccttgtgtgtttaaaaagcttgagcttttatttttctgttggagaccagagtttgatggcttgtgtgtgtgtgttttgttcttttttttttttccattgtgtcttgtcaaccccccgtttcccctcctgctgccccccatttcctacagaacgacctgcagcaataccattgacctgccgatgtccccccgcactttagattcattgatgcagtttggaaataatggtgaaggtgctgaaccctcagcaggaggacagtttgagagccttacgtttgatatggagttgacctcggagtgcgctacctcccccatgtgagcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacggcttgattatgatcatgactaaagaatgtggttagagacagtctgaggaaatgttttctgactttgttttggtttccaaccagagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggtaaaggatgaaagaagcttttcctttctttctcgaaagctagattgaattctgatcttaactgcaggcccacagaattggtactatatctccaacgtggggacttttccatattcaaatttagcccaagaattaaagtttttactttatttcggccaggcgctgtggctcacacctgtaatcccagcactttgggaga

SEQ ID NO: 45 - DNA, 2921 bp, artificial
  STAT3  DNA repair template intron 7 sequence 5
gtcaaggagtattccctcaggtcaaggagttttttcttccttcgcagacatgcaagatctgaatggaaacaaccagtcagtgaccaggcagaagatgcagcagctggaacagatgctcactgcgctggaccagatgcggagagtaagggcataggtcggaccacttcccccatgtgtctcgctcacttgcgggatttcagcgtcttgtggcagaacttgcttggtttctaagaagttcctgctctggagttgatacaagctaggcccttttgctaatcatgttcatactgtcccttttttttcctcagagcatcgtgagtgagctggctgggttgctttcagctatggaatatgtgcaaaagacgttgacagacgaggaactggccgactggaagcggaggcaacaaattgcttgcattggaggaccacccaatatatgcctggatagattggagaactggataacgtcattagcagaaagccaactgcaaactagacagcaaataaagaagctggaagaactccagcaaaaagtttcctataagggtgaccctatagtgcagcacaggcccatgttggaggaacgcattgtggagttgtttagaaatctgatgaaaagtgcctttgtggtggagcgacaaccgtgcatgcctatgcaccctgaccggcccttggtgataaagaccggagtacaattcactactaaagtgaggctgctcgtaaagttccctgaactcaattaccagttgaagataaaagtgtgcatcgataaagactctggggacgttgcagctctcagaggatcccggaagttcaacatcttgggcaccaacacaaaagtgatgaacatggaagaatccaacaacggcagcctctctgccgaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgaacaattgacgaccctggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaaggatacatcatgggctttatcagtaaggagcgggagagagcgatcctgtcaactaaacctccaggcaccttcctgctaagattcagtgaaagcagcaaagaaggaggcgtcactttcacttgggtggagaaggacatcagcggaaagacgcaaattcagagcgtggaaccatacacaaagcagcagctgaacaacatgtcatttgctgaaatcatcatgggctataagatcatggatgctaccaatatcctggtgtctccactggtctatctctatccggacattcccaaagaggaggcgttcggaaagtactgtcgcccggagtcacaggagcatcctgaagctgacccaggcagcgctgccccatacctgaagaccaagtttatctgtgtgacaccgtaagtggcttcctttccccgttttgccttcatttctaatatcctcagttatccctgggaatgggacactgggtgagagttaatctgccaaaggttggaagcccctgggctatgtttagtactcaaagtgaccttgtgtgtttaaaaagcttgagcttttatttttctgttggagaccagagtttgatggcttgtgtgtgtgtgttttgttcttttttttttttccattgtgtcttgtcaaccccccgtttcccctcctgctgccccccatttcctacagaacgacctgcagcaataccattgacctgccgatgtccccccgcactttagattcattgatgcagtttggaaataatggtgaaggtgctgaaccctcagcaggaggacagtttgagagccttacgtttgatatggagttgacctcggagtgcgctacctcccccatgtgagcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacggcttgattatgatcatgactaaagaatgtggttagagacagtctgaggaaatgttttctgactttgttttggtttccaaccagagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggtaaaggatgaaagaagcttttcctttctttctcgaaagctagattgaattctgatcttaactgcaggcccacagaattggtactatatctccaacgtggggacttttccatattcaaatttagcccaagaattaaagtttttactttatttcggccaggcgctgtggctcacacctgtaatcccagcactttgggaga

SEQ ID NO: 46 - DNA, 246 bp, artificial
  SV40polyA
gcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacggcttgattatgatca

SEQ ID NO: 47 - DNA, 2791 bp, artificial
  STAT3  DNA repair template intron 9 sequence
gtctccatgtcttcagtatttccttccccttctccatctcacctgtatacattcactttggtaattagcatctttcttaatttattggcaggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaaggtaatttagcatccttgtccctttccctcatctaaaaaatacctaaagactcacgtggtagagtgagaggcgggctgacttctggtcatggccgtggcgtgatacaagctaggcccttttgctaatcatgttcatactgtcccttttttttccacagtgcctttgtggtggagcgacaaccgtgcatgcctatgcaccctgaccggcccttggtgataaagaccggagtacaattcactactaaagtgaggctgctcgtaaagttccctgaactcaattaccagttgaagataaaagtgtgcatcgataaggactccggcgatgttgccgcgctccggggaagtcgaaagttcaacatcttgggcaccaatacaaaggtgatgaacatggaggaatcaaacaacgggtccctttccgcggagttcaaacatttgacattgcgcgagcagagatgtgggaacgggggaagagcaaattgtgacgcttctctgatcgtaactgaggagctgcatctcataacattcgagactgaggtctaccatcagggattgaagatcgacctcgaaactcattcactgcctgtagttgttattagtaatatttgccagatgccgaatgcgtgggctagcatactttggtataacatgcttacgaataatccaaagaacgttaattttttcactaaacctccaatcggaacttgggaccaggtcgcagaagtgctgagctggcaattttcctccaccaccaaacgcggtcttagcatcgaacaattgacgaccctcgcagaaaagcttttgggtcctggcgttaattactcagggtgtcagataacttgggcaaagttctgcaaagaaaacatggcaggtaagggcttttcattttgggtctggctggataacattatagatttggtgaaaaagtacattcttgccctgtggaacgaaggctacatcatgggtttcattagcaaggagcgcgagagagcgatcctgtcaactaaaccccctggaacgtttctccttagattttccgaaagcagcaaggaaggtggcgtgactttcacgtgggttgaaaaggacatcagcggaaagacgcaaattcagagcgttgaaccttacaccaagcagcaacttaacaacatgagcttcgccgagataataatgggttacaaaataatggacgctactaatatcctcgtctcacccctcgtgtacttgtatccagacattcccaaagaggaggcgttcgggaagtactgtcgcccggagtcacaagaacatcctgaagctgacccaggaagtgcagctccgtaccttaagaccaagtttatctgtgtgacaccgtaagtggcttcctttccccgttttgccttcatttctaatatcctcagttatccctgggaatgggacactgggtgagagttaatctgccaaaggttggaagcccctgggctatgtttagtactcaaagtgaccttgtgtgtttaaaaagcttgagcttttatttttctgttggaagaccagagtttgatggcttgtgtgtgtgtgttttgttcttttttttttttccattgtgtcttgtcaaccccccgtttcccctcctgctgccccccatttcctacagaacgacctgcagcaataccattgacctgccgatgtccccccgcactttagattcattgatgcagtttggaaataatggtgaaggtgctgaaccatccgctggtggacagtttgagagccttacgtttgatatggagttgacaagcgagtgtgctacatcacctatgtgagcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtacggcttgattatgatcacgtgagcccatcttctctttcctcagtgcctttgtggtggagcggcagccctgcatgcccatgcatcctgaccggcccctcgtcatcaagaccggcgtccagttcactactaaagtcaggtaggccatgccacttccatttccagtagagattttactgagggacactgttagggtgagggtagagttggtggccagggtcattctttccaggtgtggtgtcacaggcagtacactgttgcggggttgaaatttgttgccatactatctgcttgctctctgattctgatgtcaaaagcaaaagagcagtcatctttttgaaggtacctgggcatattcctatgattgtagacctggagtctcaggccacagcttctccttctgcccaagggacaaaataatgtcatctattttctgttctttgaggctactcttccctgtggattttaagggaaagagtaaggcttagtgatggggaagctgagaggccccagggcaggt

SEQ ID NO: 48 - DNA, 20 bp, artificial
  oligo 2628
agtcagtgaccaggcagaag

SEQ ID NO: 49 - DNA, 24 bp, artificial
  oligo 2852
cagttttctagccgatctaggcag

SEQ ID NO: 50 - DNA, 20 bp, artificial
  oligo 4961
ctcttcatgcaaggggatgc

SEQ ID NO: 51 - DNA, 20 bp, artificial
  oligo 5029
ccaccaccactcccggataa

SEQ ID NO: 52 - DNA, 28 bp, artificial
  oligo 3036
cttctccatctcacctgtatacattcac

SEQ ID NO: 53 - DNA, 21 bp, artificial
  oligo 4206
agaatgaccctggccaccaac

SEQ ID NO: 54 - DNA, 20 bp, artificial
  oligo 2629
tttctgcacgtactccatcg

SEQ ID NO: 55 - DNA, 24 bp, artificial
  oligo 5480
catatctgggctcagatgcttgtc

SEQ ID NO: 56 - DNA, 21 bp, artificial
  oligo 5482
tcaacttcagacccgtcaaca

SEQ ID NO: 57 - DNA, 20 bp, artificial
  oligo 5483
aactggacgccggtcttgat

SEQ ID NO: 58 - DNA, 20 bp, artificial
  oligo 4963
tgacattcccaaggaggagg

SEQ ID NO: 59 - DNA, 22 bp, artificial
  oligo 5037
caaaggtgagggactcaaactg

SEQ ID NO: 60 - DNA, 20 bp, artificial
  oligo 5325
agtactgtcgcccggagtca

SEQ ID NO: 61 - DNA, 24 bp, artificial
  oligo 5326
tcaaacgtaaggctctcaaactgt

SEQ ID NO: 62 - DNA, 20 bp, artificial
  oligo 5526
aaggagcctggtcattaagg

SEQ ID NO: 63 - DNA, 23 bp, artificial
  oligo 5728
catgattagcaaaagggcctagc

SEQ ID NO: 64 - DNA, 24 bp, artificial
  oligo 5729
cctctacaaatgtggtacggcttg

SEQ ID NO: 65 - DNA, 19 bp, artificial
  oligo 5722
tgatccgcccatcttggtc

SEQ ID NO: 66 - DNA, 25 bp, artificial
  oligo 5726
ccttgttcttattgtagtggtctcc

SEQ ID NO: 67 - DNA, 22 bp, artificial
  oligo 5725
cctgagcctcctgagagattac

SEQ ID NO: 68 - DNA, 19 bp, artificial
  oligo 5341
aacacatgctgtgcagctg

SEQ ID NO: 69 - DNA, 23 bp, artificial
  oligo 5730
cttgggcaacataacaagacacc

SEQ ID NO: 70 - DNA, 20 bp, artificial
  oligo 5485
tggctgcagtctgtagaagg

SEQ ID NO: 71 - DNA, 20 bp, artificial
  oligo 3035
tggagtctggctgtagccca

SEQ ID NO: 72 - DNA, 24 bp, artificial
  oligo 4025
gtcctgtttctccttgtcctcagt

SEQ ID NO: 73 - DNA, 20 bp, artificial
  oligo 4965
attgctgcaggtcgttggtg

SEQ ID NO: 74 - DNA, 24 bp, artificial
  oligo 4964
tccaaactgcatcaatgaatggtg

SEQ ID NO: 75 - DNA, 19 bp, artificial
  oligo 4428
cttcgattcgggaccagcc

SEQ ID NO: 76 - DNA, 17 bp, artificial
  oligo 4429
cggagccagcgtggatc

SEQ ID NO: 77 - DNA, 18 bp, artificial
  oligo 4406
ccctggcgtcgtgattag

SEQ ID NO: 78 - DNA, 23 bp, artificial
  oligo 4407
catgaggaataaacaccctttcc

(19) United States
(12) Patent Application Publication
(10) Pub. No.: US 2024/0299453 A1
(43) Pub. Date: Sep. 12, 2024

(54) GENE THERAPY FOR THE TREATMENT OF HYPER-IgE SYNDROME (HIES) BY TARGETED GENE INTEGRATION

(71) Applicants: CELLECTIS S.A., Paris (FR); ALBERT-LUDWIGS-UNIVERSITÄT FREIBURG, Freiburg (DE)

(72) Inventors: Toni Cathomen, Freiburg (DE); Tatjana Cornu, Freiburg (DE); Viviane Dettmer-Monaco, Freiburg (DE); Simone Haas, Freiburg (DE); Julia Rositzka, Freiburg (DE); Philippe Duchateau, Paris (FR); Alexandre Juillerat, Paris (FR)

(21) Appl. No.: 18/562,536

(22) PCT Filed: May 20, 2022
(86) PCT No.: PCT/EP2022/063762

§ 371 Date: Nov. 20, 2023

(30) Foreign Application Priority Data
May 20, 2021 (DK) PA202170257

(51) Int. Cl. A61K 35/17; A61K 48/00; C12N 5/0783; C12N 9/22; C12N 15/86; C12N 15/90

(52) U.S. Cl. CPC A61K 35/17; A61K 48/005; C12N 5/0636; C12N 9/22; C12N 15/86; C12N 15/907; C12N 2501/2302; C12N 2501/51; C12N 2501/515; C12N 2501/53; C12N 2510/00; C12N 2750/14143; C12N 2800/80; C12N 2830/42

(57) ABSTRACT

The present invention generally relates to the field of genome engineering (gene editing), and more specifically to gene therapy for the treatment of Hyper-lgE syndrome (HIES). In particular, the present invention provides means and methods for genetically modifying HSCs or T-cells involving gene editing reagents, such as TALE-nucleases, that specifically target an endogenous STATS gene comprising at least one mutation causing Hyper-lgE syndrome (HIES), thereby allowing the restoration of the normal cellular phenotype. The present invention also provides populations of engineered HSCs or T-cells which comprise cells comprising an exogenous polynucleotide sequence comprising at least a partial or complete sequence of a functional STATS gene, said exogenous polynucleotide sequence being integrated in an endogenous STATS gene comprising at least one mutation causing Hyper-lgE syndrome (HIES), resulting in the expression of a functional STATS polypeptide. The present invention further provides pharmaceutical compositions comprising the cell populations of the invention, and their use in gene therapy for the treatment of Hyper-lgE syndrome (HIES).

### FIELD OF THE INVENTION

The present invention generally relates to the field of genome engineering (gene editing), and more specifically to gene therapy for the treatment of Hyper-IgE syndrome (HIES). In particular, the present invention provides means and methods for genetically modifying HSCs or T-cells involving gene editing reagents, such as TALE-nucleases, that specifically target the endogenous STAT3 gene comprising at least one mutation causing Hyper-IgE syndrome (HIES), thereby allowing the restoration of the normal cellular phenotype. The present invention also provides populations of engineered HSCs or T-cells which comprise cells comprising an exogenous polynucleotide sequence comprising at least a partial or complete sequence of a functional STAT3 gene, said exogenous polynucleotide sequence being integrated in an endogenous STAT3 gene comprising at least one mutation causing Hyper-IgE syndrome (HIES), resulting in the expression of a functional STAT3 polypeptide. The present invention further provides pharmaceutical compositions comprising the cell populations of the invention, and their use in gene therapy for the treatment of Hyper-IgE syndrome (HIES).

### BACKGROUND OF THE INVENTION

Hyper-IgE syndrome (HIES) is a rare immunodeficiency characterized by recurrent skin and pulmonary abscesses and elevated levels of IgE in serum [1]. The disease is also known as Job's Syndrome and its prevalence is about 1 to 9 in 100.000. Although HIES patients have a severely reduced quality of life, allogeneic hematopoietic stem cell transplantation (HSCT) is generally not indicated because of the potential severe side effects, such as graft-versus-host disease.

Mutations leading to HIES are most frequently found in the STAT3 locus, most frequently in exons encoding the DNA binding domain or the SH2 domain (FIG. 1). They are predominantly missense mutations, resulting in single amino acid changes or short in-frame deletions [2-5]. Interestingly, nonsense mutations have not been identified, suggesting that hemizygosity is either lethal or does not lead to a phenotype. Mice with a complete deletion of a STAT3 allele are phenotypically normal [6].

The described mutations in STAT3 result in the failure of naïve T cells to differentiate into Th17 cells, with subsequent failure of IL-17 and IL-22 secretion which explains the increased susceptibility of HIES patients to infection [7-10]. Because STAT3 proteins form homo- and heterodimers with other STAT proteins upon activation, the phenotype of the above-described mutations can either be caused by a dominant-negative effect of mutated STAT3 proteins in preventing the formation of functional STAT dimers or by haploinsufficiency.

As mentioned above, STAT3 is the main mediator of IL-6-type cytokine signaling and an important transcriptional regulator of cell proliferation, maturation and survival. Moreover, STAT3 has been described as a key player in both cancer development as well as a potent tumor suppressor. This heterogeneity partially depends on its expression as different isoforms. Alternative splicing gives rise to two STAT3 isoforms, STAT3a and its truncated version STAT3β (FIG. 2A). Both isoforms are transcriptionally active and display distinct functions under physiological and pathological conditions. In fact, while STAT3α is widely described as a transcriptional activator or as an oncogene, STAT3β has gained attention as a potential tumor suppressor [11].

While gene therapy for genetic disorders with recessive inheritance generally aims at correcting or replacing the missing gene function with a gene addition type approach, diseases caused by dominant mutations or by mutations in tightly regulated loci, such as the genes encoding the JAK/STAT pathway proteins [12], require more complex strategies [13]. However, such gene therapy strategies for treating Hyper-IgE syndrome (HIES) have not been proposed yet, partly due to the difficulty of expressing STAT3α and STAT3β isoforms in a controlled and balanced manner.

Thus, there is a need for new gene therapy approaches to treat Hyper-IgE syndrome (HIES) taking into account the whole complex structure of the STAT3 gene. The present invention not only aims at correcting the mutations causing HIES but also at restoring full STAT3 gene function.

### SUMMARY OF THE INVENTION

The present invention addresses this need by providing the first gene therapy approach to treat Hyper-IgE syndrome (HIES) enabling proper STAT3α and STAT3β isoforms expression. Particularly, the present invention provides means and methods for gene editing of an endogenous STAT3 gene which comprises at least one mutation causing Hyper-IgE syndrome (HIES). As a result, populations of engineered hematopoietic stem cells (HSCs) or T-cells are provided, in which at least a partial or complete sequence of a functional STAT3 gene, in particular intron 22 has been integrated in an endogenous STAT3 gene comprising at least one mutation causing Hyper-IgE syndrome (HIES), thereby restoring the normal cellular phenotype by enabling alternative splicing and hence expression of both STAT3 isoforms.

The present invention can be further summarized by the following items:
1. A population of engineered hematopoietic stem cells (HSCs) or T-cells originating from a patient suffering from HIES, comprising cells comprising an exogenous polynucleotide sequence comprising at least a partial or complete sequence of a functional STAT3 gene, said exogenous polynucleotide sequence being integrated in an endogenous STAT3 gene comprising at least one mutation causing Hyper-IgE syndrome (HIES), resulting in the expression of a functional STAT3 polypeptide.
2. The population of engineered HSCs or T-cells according to item 1, wherein said exogenous polynucleotide sequence integrated into said endogenous STAT3 gene comprises at least one exon selected from Exons 8 to 24 of STAT3 encoding the amino acid sequence of SEQ ID NOs: 2 to 18, respectively.
3. The population of engineered HSCs or T-cells according to item 1 or 2, wherein said exogenous polynucleotide sequence integrated into said endogenous STAT3 gene comprises at least one exon selected from Exons 8 to 22 of STAT3 encoding the amino acid sequence of SEQ ID NOs: 2 to 16, respectively.
4. The population of engineered HSCs or T-cells according to item 3, wherein said exogenous polynucleotide sequence integrated into said endogenous STAT3 gene further comprises at least Exon 23 of STAT3 encoding the amino acid sequence of SEQ ID NO: 17, optionally further comprising Exon 24 of STAT3 encoding the amino acid sequence of SEQ ID NO: 18.
5. The population of engineered hematopoietic stem cells (HSCs) or T-cells according to item 4, wherein said exogenous polynucleotide sequence comprises Intron 22 of STAT3 according to SEQ ID NO: 27, which is located upstream of Exon 23 and enables an alternative splicing to Exon 23.
6. The population of engineered hematopoietic stem cells (HSCs) or T-cells according to any one of items 1 to 5, wherein said exogenous polynucleotide sequence has been inserted into an intron sequence of the endogenous STAT3 gene.
7. The population of engineered hematopoietic stem cells (HSCs) or T-cells according to item 6, wherein said exogenous polynucleotide sequence has been inserted into an intron of the endogenous STAT3 gene selected from Intron 7 (SEQ ID NO:32), Intron 8 (SEQ ID NO:33) or Intron 9 (SEQ ID NO:34).
8. The population of engineered hematopoietic stem cells (HSCs) or T-cells according to any one of items 1 to 7, wherein said exogenous polynucleotide sequence comprises in consecutive order at least Exons 8 to 22 of STAT3, Intron 22 of STAT3 and Exons 23 to 24 of STAT3, wherein Exons 8 to 24 encode the amino acid sequences of SEQ ID NOs: 2 to 18, respectively.
9. The population of engineered hematopoietic stem cells (HSCs) or T-cells according to item 8, wherein said exogenous polynucleotide sequence has been inserted into Intron 7 of the endogenous STAT3 gene.
10. The population of engineered hematopoietic stem cells (HSCs) or T-cells according to any one of items 1 to 7, wherein said exogenous polynucleotide sequence comprises in consecutive order at least Exons 9 to 22 of STAT3, Intron 22 of STAT3 and Exons 23 to 24 of STAT3, wherein Exons 9 to 24 encode the amino acid sequences of SEQ ID NOs: 3 to 18, respectively.
11. The population of engineered hematopoietic stem cells (HSCs) or T-cells according to item 10, wherein said exogenous polynucleotide sequence has been inserted into Intron 8 of the endogenous STAT3 gene.
12. The population of engineered hematopoietic stem cells (HSCs) or T-cells according to any one of items 1 to 7, wherein said exogenous polynucleotide sequence comprises in consecutive order at least Exons 10 to 22 of STAT3, Intron 22 of STAT3 and Exons 23 to 24 of STAT3, wherein Exons 10 to 24 encode the amino acid sequences of SEQ ID NOs: 4 to 18, respectively.
13. The population of engineered hematopoietic stem cells (HSCs) or T-cells according to item 12, wherein said exogenous polynucleotide sequence has been inserted into Intron 10 of the endogenous STAT3 gene.
14. The population of engineered hematopoietic stem cells (HSCs) or T-cells according to any one of items 1 to 13, wherein said partial or complete sequence of functional STAT3 comprised by the exogenous polynucleotide sequence is codon optimized.
16. The population of engineered hematopoietic stem cells (HSCs) or T-cells according to any one of items 1 to 15, wherein said exogenous polynucleotide sequence comprises upstream of the partial or complete sequence of functional STAT3 an artificial splice site, such as the artificial splice site set forth in SEQ ID NO: 28 or SEQ ID NO: 29.
17. The population of engineered hematopoietic stem cells (HSCs) or T-cells according to any one of items 1 to 16, wherein said exogenous polynucleotide sequence comprises a sequence encoding a functional STAT3 polypeptide.
18. The population of engineered hematopoietic stem cells (HSCs) or T-cells according to item 17, wherein said functional STAT3 polypeptide has at least 80%, preferably at least 90%, more preferably at least 95% and even more preferably at least 99% sequence identity with SEQ ID NO:1.
19. The population of engineered hematopoietic stem cells (HSCs) or T-cells according to item 17, wherein said functional STAT3 polypeptide comprises the amino acid sequence of SEQ ID NO:1.
20. The population of engineered hematopoietic stem cells (HSCs) or T-cells according to any one of items 1 to 19, wherein said exogenous polynucleotide sequence allows the expression of STAT3α and STAT3ß by said engineered cells.
21. The population of engineered hematopoietic stem cells (HSCs) or T-cells according to any one of items 1 to 20, wherein said engineered cells express STAT3alpha (SEQ ID NO:19) and STAT3beta (SEQ ID NO:20).
22. The population of engineered hematopoietic stem cells (HSCs) or T-cells according to item 20 or 21, wherein STAT3alpha has at least 80%, preferably at least 90%, more preferably at least 95% and even more preferably at least 99% sequence identity with SEQ ID NO:19 and STAT3beta has at least 80%, preferably at least 90%, more preferably at least 95% and even more preferably at least 99% sequence identity with SEQ ID NO:20.
23. The population of engineered hematopoietic stem cells (HSCs) or T-cells according to any one of items 1 to 18, wherein said STAT3alpha comprises the amino acid sequence of SEQ ID NO:19 and said STAT3beta comprises the amino acid sequence SEQ ID NO:20.
24. The population of engineered hematopoietic stem cells (HSCs) or T-cells according to any one of items 1 to 23, wherein said engineered cells express STAT3alpha and STAT3beta isoforms in a ratio from about 3:1 to about 7:1, or comprised from about 4:1 to about 6:1, such as about 4:1 or about 5:1 (STAT3α:STAT3β).
25. The population of engineered hematopoietic stem cells (HSCs) or T-cells according to any one of items 1 to 24, wherein said exogenous polynucleotide sequence has been inserted by site-directed gene integration by using a sequence-specific reagent inducing DNA cleavage, such as rare-cutting endonuclease or nickase.
26. The population of engineered hematopoietic stem cells (HSCs) or T-cells according to any one of items 1 to 25, wherein said exogenous polynucleotide sequence is integrated by homologous recombination or by non-homologous end-joining (NHEJ).
27. The population of engineered hematopoietic stem cells (HSCs) or T-cells according to any one of items 1 to 24, wherein the partial or complete sequence of functional STAT3 gene has been inserted under the transcriptional control of the endogenous STAT3 promoter.
28. The population of engineered hematopoietic stem cells (HSCs) or T-cells according to any one of items 1 to 27, wherein said endogenous STAT3 gene comprises at least one mutation mentioned in FIG. 1.
29. The population of engineered hematopoietic stem cells (HSCs) or T-cells according to any one of items 1 to 28, wherein said endogenous STAT3 gene comprises at least one mutation selected from the group consisting of C328_P330dup, H332Y, H332L, R335W, K340E, G342D, c. 1020delGAC, V343F, V343L, D369del, c.110-1G>a, c. 110-1G>G, c.1139+1G>T, c.1139+1G>A, c.1139+2insT, c.1140-2A>C, R382W, R382L, R382Q, F384S, F384L, T3891, T412A, R423Q, R432M, H437P, H437Y, V463del, S465A, N466D, N466S, N466T, N466K, Q469H, Q469R, N472D, K531E, 1568F, K591E, S611N, S611I, S611G, S614G, G617E, G617V, T620A, F621V, S636Y, V637M, V637L, V637A, V638G, P639A, P639, Y640N, K642E, Q644P, Q644del, N647D, E652K, Y657C, Y657N, Y657S, M660T, M663S, 1665N, S668F, S668Y, E690_P699del, Y705H, L706P, L706M, T708S, T708N, K709E, F710C, I711T, V713M, V713L, T7141, T714A, and c.2144+1G>A.
30. The population of engineered hematopoietic stem cells (HSCs) or T-cells according to any one of items 1 to 29, wherein said HSCs or T-cells are primary cells.
31. The population of engineered hematopoietic stem cells (HSCs) according to any one of items 1 to 30, wherein said cells are CD34+.
32. The population of engineered T-cells according to any one of items 1 to 30, wherein said T-cells are CD4+ or CD8+.
33. The population of engineered T-cells according to any one of items 1 to 30 and 32, wherein said T-cells comprise at least 1%, such as at least 10% of long-lived T-cell, such as naive T-cells (Th0), effector memory (TEM), central memory (TCM), and stem cell memory (TSCM) T-cells.
34. A pharmaceutical composition comprising a population of cells according to any one of claims 1 to 33, and a pharmaceutically acceptable excipient and/or carrier.
35. The population of engineered HSCs or T-cells according to any one of items 1 to 33 or the pharmaceutical composition according to item 34 for use in the treatment of Hyper-IgE syndrome (HIES).
36. The population of engineered HSCs according to any one of items 1 to 31 or the pharmaceutical composition according to item 34 comprising a population of engineered HSCs for use in stem cell transplantation, such as bone marrow transplantation.
37. A polynucleotide donor template, such as a DNA donor template, characterized in that it comprises at least a partial or complete sequence of a functional STAT3 gene.
38. The polynucleotide donor template according to item 37, comprising at least one exon selected from Exons 8 to 24 of STAT3 encoding the amino acid sequence of SEQ ID NOs: 2 to 18, respectively.
39. The polynucleotide donor template according to item 37 or 38, comprising at least one exon selected from Exons 8 to 22 of STAT3 encoding the amino acid sequence of SEQ ID NOs: 2 to 16, respectively.
40. The polynucleotide donor template according to item 39, further comprising at least Exon 23 of STAT3 encoding the amino acid sequence of SEQ ID NO: 17, optionally further comprising Exon 24 of STAT3 encoding the amino acid sequence of SEQ ID NO: 18.
41. The polynucleotide donor template according to item 38, comprising Intron 22 of STAT3 according to SEQ ID NO: 27, which is located upstream of Exon 23 and enables an alternative splicing to Exon 23.
42. The polynucleotide donor template according to any one of items 37 to 41, comprising in consecutive order at least Exons 8 to 22 of STAT3, Intron 22 of STAT3 and Exons 23 to 24 of STAT3, wherein Exons 8 to 24 encode the amino acid sequences of SEQ ID NOs: 2 to 18, respectively.
43. The polynucleotide donor template according to any one of items 37 to 41, comprising in consecutive order at least Exons 9 to 22 of STAT3, Intron 22 of STAT3 and Exons 23 to 24 of STAT3, wherein Exons 9 to 24 encode the amino acid sequences of SEQ ID NOs: 3 to 18, respectively.
44. The polynucleotide donor template according to any one of items 37 to 41, comprising in consecutive order at least Exons 10 to 22 of STAT3, Intron 22 of STAT3 and Exons 23 to 24 of STAT3, wherein Exons 10 to 24 encode the amino acid sequences of SEQ ID NOs: 4 to 18, respectively.
45. The polynucleotide donor template according to any one of items 37 to 44, wherein said partial or complete sequence of a functional STAT3 gene is codon optimized.
46. The polynucleotide donor template according to any one of items 37 to 45, comprising upstream of the partial or complete sequence of a functional STAT3 gene an artificial splice site, such as the artificial splice site set forth in SEQ ID NO: 28 or SEQ ID NO: 29.
47. The polynucleotide donor template according to any one of items 37 to 46, comprising a sequence encoding a functional STAT3 polypeptide.
48. The polynucleotide donor template according to 47, wherein said functional STAT3 polypeptide has at least 80%, preferably at least 90%, more preferably at least 95% and even more preferably at least 99% sequence identity with SEQ ID NO: 1.
49. The polynucleotide donor template according to 47, wherein said functional STAT3 polypeptide comprises the amino acid sequence of SEQ ID NO: 1.
50. The polynucleotide donor template according to any one of items 37 to 49, allowing the expression of STAT3alpha and STAT3beta.
51. The polynucleotide donor template according to 50, wherein STAT3alpha has at least 80%, preferably at least 90%, more preferably at least 95% and even more preferably at least 99% sequence identity with SEQ ID NO: 19 and STAT3beta has at least 80%, preferably at least 90%, more preferably at least 95% and even more preferably at least 99% sequence identity with SEQ ID NO:20.
52. The polynucleotide donor template according to 50, wherein said STAT3alpha comprises the amino acid sequence of SEQ ID NO: 19 and said STAT3beta comprises the amino acid sequence SEQ ID NO: 20.
53. The polynucleotide donor template according to any one of item 37 to 52, further comprising a polyadenylation sequence, such as SV40polyA.
54. The polynucleotide donor template according to any one of items 37 to 53, further comprising left and/or right homology sequences having at least 80% sequence identity with the endogenous polynucleotide sequence encoding STAT3.
55. The polynucleotide donor template according to item 54, wherein said left and/or right homology sequence comprises a sequence having at least 80% identity with SEQ ID NO: 30, 31 or 32.
56. The polynucleotide donor template according to any one of items 37 to 55, comprising a polynucleotide sequence having at least 70% sequence identity with SEQ ID NO: 33.
57. An AAV or IDLV vector comprising a polynucleotide donor template according to any one of items 37 to 56.
58. The AAV vector according to item 57, which is an AAV6 vector.
59. The polynucleotide donor template according to any one of items 37 to 56 or the vector according to item 57 or 58 for use in the treatment of Hyper-IgE syndrome (HIES).
60. A rare-cutting endonuclease or nickase, characterized in that it is capable of cleaving a sequence within the STAT3 gene, preferably comprised within the intron 7 polynucleotide sequence (SEQ ID NO: 30), intron 8 polynucleotide sequence (SEQ ID NO: 31) or intron 9 polynucleotide sequence (SEQ ID NO: 32).
61. The rare-cutting endonuclease according to item 60, wherein said rare-cutting endonuclease is a meganuclease, zinc finger nuclease (ZFN), TALE-nuclease, megaTAL or RNA-guided endonuclease.
62. The rare-cutting endonuclease according to item 60 or 61, wherein said rare-cutting endonuclease is a TALE-nuclease.
63. The rare-cutting endonuclease according to item 56, wherein said TALE-nuclease comprises a monomer targeting a STAT3 polynucleotide sequence selected from SEQ ID NO: 38, SEQ ID NO: 39 and SEQ ID NO: 40.
64. The rare-cutting endonuclease according to item 63, wherein said TALE-nuclease monomer has at least 80% sequence identity with the polypeptide sequence of any one of SEQ ID NOs: 21 to 26.
65. The rare-cutting endonuclease according to item 62 or 63, wherein said TALE-nuclease comprises a first monomer having at least 80% sequence identity with the polypeptide sequence of SEQ ID NO: 21 and second monomer having at least 80% sequence identity with the polypeptide sequence of SEQ ID NO: 22; a first monomer having at least 80% sequence identity with the polypeptide sequence of SEQ ID NO: 23 and second monomer having at least 80% sequence identity with the polypeptide sequence of SEQ ID NO: 24; or a first monomer having at least 80% sequence identity with the polypeptide sequence of SEQ ID NO: 25 and second monomer having at least 80% sequence identity with the polypeptide sequence of SEQ ID NO: 26.
66. A polynucleotide encoding a rare-cutting endonuclease or nickase according to any one of items 60 to 65.
67. The rare-cutting endonuclease or nickase according to any one of items 60 to 65 or the polynucleotide according to item 66 for use in the treatment of Hyper-IgE syndrome (HIES).
68. The rare-cutting endonuclease or nickase according to any one of items 60 to 65 or the polynucleotide according to item 66 for use in combination with a polynucleotide donor template according to any one of items 37 to 56 to gene edit T-cells or HSCs.
69. The rare-cutting endonuclease for use according to item 68, for its use to gene edit T-cells or HSCs ex-vivo.
70. A cell from the hematopoietic cell lineage, characterized in that it has been transfected with a polynucleotide donor template according to any one of items 37 to 56 and/or a polynucleotide according to item 66.
71. The cell according to item 70, for its use in the manufacture of a therapeutic composition or population of cells.
72. Method for engineering a population of T-cells or HSCs comprising the steps of:
   Introducing in T-cells or HSCs originating from a patient suffering from HIES a polynucleotide donor template comprising at least a partial or complete sequence of a functional STAT3 gene;
   Introducing in said T-cells or HSCs a sequence-specific reagent inducing DNA cleavage to obtain cleavage of the endogenous STAT3 gene in an intron sequence, preferably in Intron 7, 8 or 9, and inserting at this locus said polynucleotide donor template by homologous recombination or NHEJ; and
   Optionally, cultivating the cells for expression of STAT3alpha and STAT3beta isoforms.
73. The method according to item 72, wherein said polynucleotide donor template is the polynucleotide donor template according to any one of items 37 to 56.
74. The method according to item 72 or 73, wherein said polynucleotide donor template is introduced in said T-cells or HSCs via a vector according to item 51 or 52.
75. The method according to any one of items 72 to 74, wherein said sequence-specific reagent inducing DNA cleavage is a rare-cutting endonuclease or nickase according to any one of items 60 to 65.
76. A population of cells obtainable by the method according to any one of items 72 to 75.
77. Method of treating Hyper-IgE syndrome (HIES) in patient in need thereof, the method comprising administering a therapeutically effective amount of a population of HSCs or T-cells according to any one of items 1 to 33, or a pharmaceutical composition according to item 34 to said patient.
78. Method of treating Hyper-IgE syndrome (HIES) in a patient in need thereof by heterologous expression of at least a partial or complete sequence of a functional STAT3 gene, wherein said heterologous partial or complete STAT3 gene sequence restores functional expression of STAT3alpha and STAT3beta isoforms in T-cells.
79. The method according to item 78, wherein the partial or complete STAT3 gene sequence is comprised in an exogenous polynucleotide sequence as defined in any one of items 1 to 25.
80. The method according to item 79, wherein said functional expression of STAT3 isoforms allows a ratio from about 3:1 to about 7:1, or from about 4:1 to 6:1, such as about 4:1 or about 5:1 (STAT3α:STAT3β).
81. Method of treating Hyper-IgE syndrome (HIES) in a patient in need thereof by gene therapy, wherein said gene therapy comprises introducing into HSCs or T-cells at least one corrected STAT3 exon sequence into an endogenous genomic STAT3 sequence by targeted gene integration.
82. The method according to item 81, wherein said HSCs or T-cells originate from the patient.
83. The method according to item 81 or 82, wherein said gene therapy comprises introducing into HSCs or T-cells at least one exogenous STAT3 gene sequence as defined in any one of items 1 to 25.

### BRIEF DESCRIPTION OF THE FIGURES

FIG. 1: Hyper IgE Syndrome-causing STAT3 mutations. Schematic overview of the STAT3 gene locus and known disease-causing mutations. Exon regions 1-24 are depicted as boxes, functional regions of the STAT3 gene are highlighted in different shades of grey. All known STAT3 mutations with the main cluster spanning exons 10-24 are listed, with black lines referring back to the region in the genome.

FIG. 2: Gene editing strategy. A) STAT3 locus and mRNA. STAT3 exons 1-24 are depicted as boxes, intronic sequences are shown as connective lines between them. Alternative splicing of exons 22-23 to produce the STAT3α (grey box segments) and STAT3β (black box segments) isoforms is highlighted. Exons 7-10 are zoomed out to highlight the three different TALEN target sites. B) AAV6 donor vector design. The three corresponding donors are shown with arrowed boxes indicating each encoded STAT3 exon. Integration is mediated by left and right homology arms (HA) flanking the donor template that are specific for each construct. Cleavage and donor integration will generate a full length STAT3 transcript which contains the alternative splice donor and splice acceptor sites required for the expression of STAT3α and STAT3ß isoforms.

FIG. 3: Assessment of indel frequencies. A-B) T7E1 assay results from untreated (−) or TALEN i7, TALEN i8 or TALEN i9 treated (+) T-cells. Shown are representative agarose gels (A) or a bar plot (B). C) Genotyping by NGS of indicated TALEN treated (+) or untreated (−) T cells. Shown is results of NGS analyses by bar plot.

FIG. 4: Assessment of targeted integration and relative transgene expression. A-B) Detection of targeted integration induced by TALEN i7 or TALEN i9 alone or with increasing amounts of their corresponding donors. In/out-PCR was assessed qualitatively by agarose gel electrophoresis (A) and quantitatively by ddPCR (B). C-D) STAT3 transgene-specific mRNA expression in indicated edited cells. RT-PCR was assessed qualitatively by agarose gel electrophoresis (C) and quantitatively by RT-ddPCR (D). Transgene expression is displayed relative to endogenous expression of STAT3α and STAT3β. E) STAT3β/a ratios: STAT3β/a mRNA ratios from transgene and endogene quantified by RT-ddPCR. Confidence intervals (CI) of B/a ratios were determined based on endogenous mRNA expression in untreated cells. E: endogene, ST: STAT3 Transgene.

FIG. 5: Characterization of PBMCs of three Hyper IgE Syndrome patients. A) Relative STAT3p/STAT3 protein expression after IL-6 treatment (+) or not (−) on PBMCs from Healthy donor (HD) or from Hyper IgE Syndrome patients with mutations V637M, R382W and K340E. B) SOCS3 expression after IL-6 treatment (+) or not (−) on PBMCs from HD, V637M, R382W and K340E. C-F) Release of cytokines IL-17 (C), IFNγ (D), TNF (E) and IL-10 (F) after PMA/lonomycin stimulation (+) or not (−) in PBMCs from HD, V637M, R382W and K340E. Numbers in graph is the fold increase relative to the unstimulated control.

FIG. 6: Validation of gene editing approach in PBMCs of three Hyper IgE Syndrome patients. A-B) T7E1 assay results on TALEN-edited (+) PBMCs from HD, V637M, R382W and K340E. Untreated controls (−). Shown are a representative agarose gel (A) and a bar plot (B). C) Targeted integration efficiency measured by quantitative in/out-ddPCR in untreated, TALENi7 treated, and TALENi7/AAVi7 treated PBMCs from HD, V637M, R382W and K340E. D) STAT3 transgene-specific mRNA expression. Transgene expression is displayed relative to endogenous expression of STAT3α and STAT3β. E) T cell subset analysis in untreated, TALENi7 treated, and TALENi7/AAVi7 treated PBMCs from HD, V637M, R382W and K340E, after or not restimulation (CD2/CD3/CD28). Shown are the percentages of effector (T_{eff}+Tₑₘ) to central memory (T_{cm}+T_{scm}) phenotypes.

### DETAILED DESCRIPTION OF THE INVENTION

Unless specifically defined herein, all technical and scientific terms used herein have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, genetics, transgenic biology, microbiology, recombinant DNA, and immunology, which are within the skill of the art. Such techniques are explained fully in the literature. See, for example, Current Protocols in Molecular Biology [Frederick M. AUSUBEL (2000) Wiley and son Inc, Library of Congress, USA; Molecular Cloning: A Laboratory Manual, Third Edition] [Sambrook et al (2001) Cold Spring Harbor, New York: Cold Spring Harbor Laboratory Press]; Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

### Cells and Cell Populations of the Present Invention

To the inventor's knowledge, the present invention is the first gene therapy approach to treat Hyper-IgE syndrome (HIES) enabling proper STAT3α and STAT3β isoforms expression. Particularly, the present invention provides means and methods for gene editing of an endogenous STAT3 gene which comprises at least one mutation causing Hyper-IgE syndrome (HIES). As a result, populations of engineered hematopoietic stem cells (HSCs) or T-cells are provided, which comprise cells comprising an exogenous polynucleotide sequence comprising at least a partial or complete sequence of a functional STAT3 gene, said exogenous polynucleotide sequence being integrated in an endogenous STAT3 gene comprising at least one mutation causing Hyper-IgE syndrome (HIES), resulting in the expression of a functional STAT3 polypeptide.

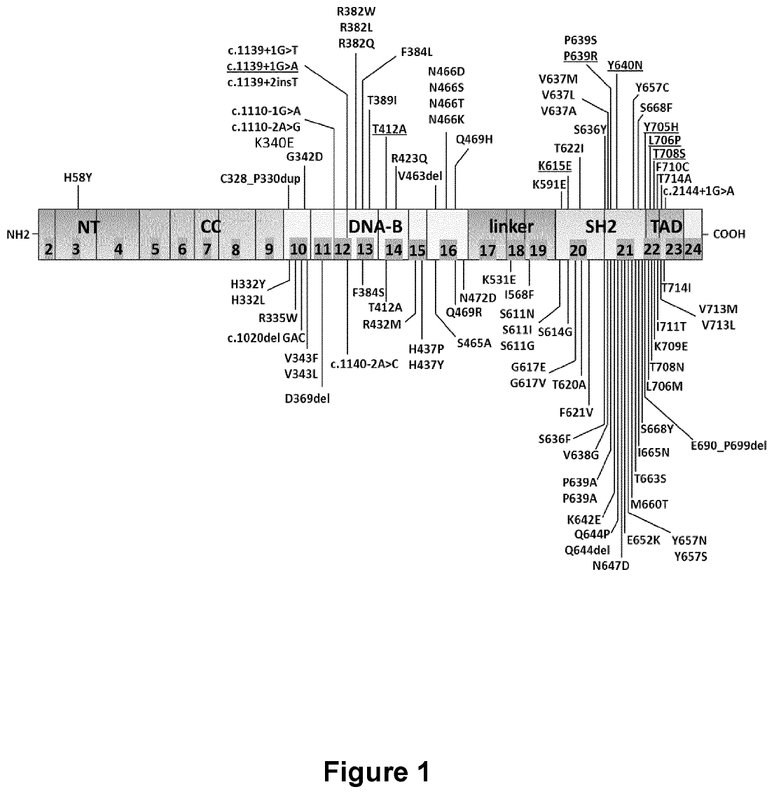

Given that most mutations leading to HIES affect primarily the DNA binding and the SH2 domains (FIG. 1), the present invention particularly aims at replacing the defective STAT3 gene sequence downstream of Intron 7 of the endogenous STAT3 gene in HSCs or T-cells of a patient affected by HIES by a corrective STAT3 sequence, thereby restoring the normal cellular phenotype by enabling alternative splicing and hence expression of both STAT3 isoforms. Generally, the corrective STAT3 sequence should include at least those exons of STAT3 which have been identified as exons harboring HIES causing mutations in the endogenous STAT3 gene of the HIES patient. By way of example, a corrective STAT3 sequence within the meaning of the present invention may include at least one exon selected from Exons 8 to 24 of STAT3 encoding the amino acid sequence of SEQ ID NOs: 2 to 18, respectively. The corrective STAT3 sequence may, for example, encompass at least Exons 8 to 24, at least Exons 9 to 24 or at least Exons 10 to 24, respectively, as well as Intron 22 (280 bp) to enable alternative splicing to Exon 23. Yet, the presence of other introns of STAT3, notably those adjacent to the respective exons, is not excluded. Such further introns may also be included in the corrective STAT3 sequence should this be preferred.

While the present invention contemplates targeted integration of a corrective STAT3 sequence at any intron within the endogenous STAT3 gene, most suitable sites for targeted integration will be Intron 7, 8 or 9 of STAT3. Such an approach allows the treatment of the great majority of HIES patients using a single approach, as all disease-causing mutations downstream of Intron 7 can be corrected with the very same strategy and tools, respectively. Based on clinical observations, the targeted integration of the corrective STAT3 sequence (e.g., exons8-22-intron22-exons23-24) will restore the normal cellular phenotype by enabling alternative splicing and hence expression of both STAT3 isoforms. While allele-specific gene disruption will represent a highly individualized treatment approach, the targeted integration of at least a partial, intron-containing exogenous STAT3 gene sequence into a selected endogenous STAT3 intron represents a powerful treatment option that will serve as paradigm for treating most STAT3 disorders caused by mutations in this tightly regulated gene.

Thus, in a general aspect, the present invention is drawn to a population of engineered hematopoietic stem cells (HSCs) or T-cells which comprises cells comprising an exogenous polynucleotide sequence comprising at least a partial or complete sequence of a functional STAT3 gene, said exogenous polynucleotide sequence being integrated in an endogenous STAT3 gene comprising at least one mutation causing Hyper-IgE syndrome (HIES), resulting in the expression of a functional STAT3 polypeptide.

More specifically, the present invention is drawn to a population of engineered hematopoietic stem cells (HSCs) or T-cells originating from a patient suffering from HIES, comprising cells comprising an exogenous polynucleotide sequence comprising at least a partial or complete sequence of a functional STAT3 gene, said exogenous polynucleotide sequence being integrated in an endogenous STAT3 gene comprising at least one mutation causing Hyper-IgE syndrome (HIES), resulting in the expression of a functional STAT3 polypeptide.

Preferably, at least 10% of the total cells of the cell population are cells comprising an exogenous polynucleotide sequence comprising at least a partial or complete sequence of a functional STAT3 gene, said exogenous polynucleotide sequence being integrated in an endogenous STAT3 gene comprising at least one mutation causing Hyper-IgE syndrome (HIES), resulting in the expression of a functional STAT3 polypeptide.

According to some embodiments, at least 20%, such as at least 30% or at least 40%, of the total cells of the cell population are cells comprising an exogenous polynucleotide sequence comprising at least a partial or complete sequence of a functional STAT3 gene, said exogenous polynucleotide sequence being integrated in an endogenous STAT3 gene comprising at least one mutation causing Hyper-IgE syndrome (HIES), resulting in the expression of a functional STAT3 polypeptide.

According to some embodiments, at least 50%, such as at least 60% or at least 70%, of the total cells of the cell population are cells comprising an exogenous polynucleotide sequence comprising at least a partial or complete sequence of a functional STAT3 gene, said exogenous polynucleotide sequence being integrated in an endogenous STAT3 gene comprising at least one mutation causing Hyper-IgE syndrome (HIES), resulting in the expression of a functional STAT3 polypeptide.

According to some embodiments, at least 80%, such as at least 90% or at least 95%, of the total cells of the cell population are cells comprising an exogenous polynucleotide sequence comprising at least a partial or complete sequence of a functional STAT3 gene, said exogenous polynucleotide sequence being integrated in an endogenous STAT3 gene comprising at least one mutation causing Hyper-IgE syndrome (HIES), resulting in the expression of a functional STAT3 polypeptide.

With “functional STAT3 gene” it is meant an assembly of genetic sequences either under DNA or RNA form, that concurs to the expression of a functional human STAT3 polypeptide, preferably a STAT3 polypeptide of SEQ ID NO: 1, and more specifically the two STAT3 isoforms STAT3alpha (SEQ ID NO: 19) and STAT3beta (SEQ ID NO: 20). The “functional STAT3 gene” does not contain any mutations which would cause a disease state such as Hyper-IgE syndrome (HIES), but rather provides for a normal cellular phenotype by enabling alternative splicing and hence expression of both STAT3 isoforms.

With “functional STAT3 polypeptide” it is meant a STAT3 polypeptide as occurring in the human population in healthy subjects, such as the representative STAT3 sequence of SEQ ID NO: 1, and more specifically the two STAT3 isoforms STAT3alpha (SEQ ID NO: 19) and STAT3beta (SEQ ID NO:20). The definition of functional STAT3 polypeptide encompasses human variants of SEQ ID NO: 1 having at least 80%, preferably at least 90%, more preferably at least 95% and even more preferably at least 99% sequence identity with SEQ ID NO:1, while retaining an equivalent effect on Th17 cells differentiation.

According to some embodiments, the population is a population of engineered HSCs. Preferably, the population of engineered HSCs comprises at least 50%, more preferably at least 70%, and even more preferably at least 90% of CD34+ cells.

According to some embodiments, the population is a population of engineered T-cells. The T-cells may be CD4+ or CD8+. According to some embodiments, said T-cells comprise at least 1%, preferably at least 5%, more preferably at least 10%, even more preferably at least 15%, most preferably at least 20% of long-lived T-cell, such as naive T-cells (Th0), effector memory (TEM), central memory (TCM), and stem cell memory (TSCM) T-cells.

The HSCs or T-cells comprised by the population may be primary cells. Primary cells are generally used in cell therapy as they are deemed more functional and less tumorigenic. In general, primary cells can be obtained from the patient suffering from HIES through a variety of methods known in the art, as for instance by leukapheresis techniques as reviewed by Schwartz J. et al. [Guidelines on the use of therapeutic apheresis in clinical practice-evidence-based approach from the Writing Committee of the American Society for Apheresis: the sixth special issue (2013) J. Clin. Apher. 28(3): 145-284]. HSCs and progenitor cells can be taken from bone marrow, and more particularly from the pelvis, at the iliac crest, using a needle or syringe. Alternatively, HSCs may be harvested from the circulating peripheral blood, while blood donors are injected with a HSC mobilizing agent, such as granulocyte-colony stimulating factor (G-CSF) and/or plerixafor, that induces cells to leave the bone marrow and circulate in the blood vessels. HSCs may also be harvested from cord blood.

Said HSCs or T-cells are generally human cells.

According to some embodiments, said exogenous polynucleotide sequence integrated into said endogenous STAT3 gene comprises at least one exon selected from Exons 8 to 24 of STAT3 encoding the amino acid sequence of SEQ ID NOs: 2 to 18, respectively.

According to some embodiments, said exogenous polynucleotide sequence integrated into said endogenous STAT3 gene comprises at least one exon selected from Exons 8 to 22 of STAT3 encoding the amino acid sequence of SEQ ID NOs: 2 to 16, respectively.

According to some embodiments, said exogenous polynucleotide sequence integrated into said endogenous STAT3 gene comprises at least Exons 8 to 22 of STAT3 encoding the amino acid sequence of SEQ ID NOs: 2 to 16, respectively.

According to some embodiments, said exogenous polynucleotide sequence integrated into said endogenous STAT3 gene comprises at least Exons 9 to 22 of STAT3 encoding the amino acid sequence of SEQ ID NOs: 3 to 16, respectively.

According to some embodiments, said exogenous polynucleotide sequence integrated into said endogenous STAT3 gene comprises at least Exons 10 to 22 of STAT3 encoding the amino acid sequence of SEQ ID NOs: 4 to 16, respectively.

According to some embodiments, said exogenous polynucleotide sequence integrated into said endogenous STAT3 gene further comprises Exon 23 of STAT3 encoding the amino acid sequence of SEQ ID NO: 17, optionally further comprising Exon 24 of STAT3 encoding the amino acid sequence of SEQ ID NO: 18.

According to some embodiments, said exogenous polynucleotide sequence comprises Intron 22 of STAT3 according to SEQ ID NO: 27, which is located upstream of Exon 23 and enables an alternative splicing to Exon 23.

According to some embodiments, said exogenous polynucleotide sequence has been inserted into an intron sequence of the endogenous STAT3 gene.

According to some embodiments, said exogenous polynucleotide sequence has been inserted into an intron of the endogenous STAT3 gene selected from Intron 7 (SEQ ID NO: 30), Intron 8 (SEQ ID NO: 31) or Intron 9 (SEQ ID NO: 32).

According to some embodiments, said exogenous polynucleotide sequence has been inserted into Intron 7 (SEQ ID NO: 30) of the endogenous STAT3 gene.

According to some embodiments, said exogenous polynucleotide sequence has been inserted into Intron 8 (SEQ ID NO: 31) of the endogenous STAT3 gene.

According to some embodiments, said exogenous polynucleotide sequence has been inserted into Intron 9 (SEQ ID NO: 32) of the endogenous STAT3 gene.

According to some embodiments, said exogenous polynucleotide sequence comprises in consecutive order (i.e. from 5′ to 3′), at least Exons 8 to 22 of STAT3, Intron 22 of STAT3 and Exons 23 to 24 of STAT3, wherein Exons 8 to 24 encode the amino acid sequences of SEQ ID NOs: 2 to 18, respectively. Suitably, said exogenous polynucleotide sequence has been inserted into Intron 7 of the endogenous STAT3 gene.

According to some embodiments, said exogenous polynucleotide sequence comprises the polynucleotide sequence of SEQ ID NO: 33, SEQ ID NO: 34, SEQ ID NO:35, SEQ ID NO:36 or SEQ ID NO:37. Suitably, said exogenous polynucleotide sequence has been inserted into Intron 7 of the endogenous STAT3 gene.

According to some embodiments, said exogenous polynucleotide sequence comprises in consecutive order (i.e. from 5′ to 3′) at least Exons 9 to 22 of STAT3, Intron 22 of STAT3 and Exons 23 to 24 of STAT3, wherein Exons 9 to 24 encode the amino acid sequences of SEQ ID NOs: 3 to 18, respectively. Suitably, said exogenous polynucleotide sequence has been inserted into Intron 8 of the endogenous STAT3 gene.

According to some embodiments, said exogenous polynucleotide sequence comprises in consecutive order (i.e. from 5′ to 3′) at least Exons 10 to 22 of STAT3, Intron 22 of STAT3 and Exons 23 to 24 of STAT3, wherein Exons 10 to 24 encode the amino acid sequences of SEQ ID NOs: 4 to 18, respectively. Suitably, said exogenous polynucleotide sequence has been inserted into Intron 9 of the endogenous STAT3 gene.

Generally, once integrated in endogenous STAT3 gene, said exogenous polynucleotide sequence allows the expression of a functional STAT3 polypeptide, preferably of SEQ ID NO: 1, and more specifically the expression of the two STAT3 isoforms STAT3alpha and STAT3beta, by said engineered cells. Preferably, said engineered cells express STAT3alpha and STAT3beta isoforms in a ratio from about 3:1 to 7:1, or from about 4:1 to about 6:1, such as about 4:1 or about 5:1 (STAT3α:STAT3β). According to some embodiments, said engineered cells express STAT3alpha and STAT3beta isoforms is in a ratio of about 4:1 (STAT3α:STAT3β). According to some embodiments, said engineered cells express STAT3alpha and STAT3beta isoforms is in a ratio of about 5:1 (STAT3α:STAT3β). According to some embodiments, said engineered cells express STAT3alpha and STAT3beta isoforms is in a ratio of about 6:1 (STAT3α:STAT3β).

In order to improve the expression of the functional STAT3 polypeptide and to avoid homologous recombination with the endogenous STAT3 coding sequence, said partial or complete sequence of a functional STAT3 gene comprised by the exogenous polynucleotide sequence may be codon optimized. Thus, according to some embodiments, said partial or complete sequence of a functional STAT3 gene is codon optimized.

With “codon optimized” it is meant that the polynucleotide sequence has been adapted for expression in the cells of a given vertebrate, such as a human or other mammal, by replacing at least one, or more than one, or a significant number, of codons with one or more codons that are more frequently used in the genes of that vertebrate. Accordingly, the partial or complete sequence of a functional STAT3 gene can be tailored for optimal gene expression in a given organism based on codon optimization. Codon optimization can be done based on established codon usage tables, for example, at the “Codon Usage Database” available at http://www.kazusa.or.jp/codon/(hosted by Kazusa DNA Research Institute, Japan), or other kind of computer algorithms. A non-limiting example is the OptimumGene PSO algorithm from GenScript® which takes into consideration a variety of critical factors involved in different stages of protein expression, such as codon adaptability, mRNA structure, and various cis-elements in transcription and translation. By utilizing codon usage tables or other kind of computer algorithms, one of ordinary skill in the art can apply the frequencies to any given polypeptide sequence, and produce a nucleic acid fragment of a codon-optimized coding region which encodes the polypeptide, but which uses codons optimal for a given organism.

Preferably, codon optimization also includes the removal of any cryptic splicing site as well as any microRNA target site and/or a sequence which would generate a mRNA secondary structure impeding translation. Thus, according to some embodiments, said codon optimized sequence has been further optimized to remove at least one cryptic splicing site and/or to remove at least one microRNA target site and/or a sequence which would generate a mRNA secondary structure impeding translation. Computer algorithms allowing the prediction of cryptic splicing site are well-known to the skilled person, and include, for example, the splice prediction tool available at http://wangcomputing.com/assp/. Similarly, one of ordinary skill in the art can use established computer algorithms, such as the miRNA target prediction tool available at http://mirdb.org/, to identify miRNA target sites. Codon optimization also allows to remove identical polynucleotide sequences that would be prompt to unwanted genetic recombination that could interfere with the process of targeted gene integration.

The exogenous polynucleotide sequence may comprise upstream of the partial or complete sequence of functional STAT3 a natural or artificial splice site. Such splice site has the advantage of allowing easier splicing and/or better transcription/translation of downstream sequences. Thus, according to some embodiments, said exogenous polynucleotide sequence comprises upstream of the partial or complete sequence of functional STAT3 a natural or artificial splice site. According to some embodiments, said exogenous polynucleotide sequence comprises upstream of the partial or complete sequence of functional STAT3 an artificial splice site, such as such as the artificial splice site set forth in SEQ ID NO: 28 or SEQ ID NO:29.

Further, in order to allow regulation of translation of the coding sequence from mRNA, said exogenous polypeptide sequence may further comprise a 5′ untranslated region (5′ UTR) (also known as a leader sequence, transcript leader, or leader RNA) placed between said natural or artificial splice site and the partial or complete sequence of functional STAT3. A non-limiting example of such 5′UTR is set for in SEQ ID NO: 5UTR.

Preferably, the exogenous polynucleotide sequence is integrated by homologous recombination or by non-homologous end-joining (NHEJ). Moreover, the partial or complete sequence of functional STAT3 gene comprised by the exogenous polynucleotide sequence has been inserted under the transcriptional control of the endogenous STAT3 promoter.

Targeted (i.e. site-directed) integration of the exogenous polynucleotide sequence into the endogenous STAT3 gene is suitable done by using a sequence-specific reagent inducing DNA cleavage, such as a rare-cutting endonuclease or nickase. Thus, according to some embodiments, said exogenous polynucleotide sequence has been inserted by site-directed gene integration by using a sequence-specific reagent inducing DNA cleavage. Further details on sequence-specific reagents inducing DNA cleavage are given below, and apply mutatis mutandis.

As noted above, most mutations leading to HIES affect the DNA binding and the SH2 domains (FIG. 1). Thus, said endogenous STAT3 gene may comprise at least one mutation selected from the group consisting of C328_P330dup, H332Y, H332L, R335W, G342D, c. 1020delGAC, K340E, V343F, V343L, D369del, c.110-1G>a, c.110-1G>G, c.1139+1G>T, c.1139+1G>A, c.1139+2insT, c.1140-2A>C, R382W, R382L, R382Q, F384S, F384L, T3891, T412A, R423Q, R432M, H437P, H437Y, V463del, S465A, N466D, N466S, N466T, N466K, Q469H, Q469R, N472D, K531E, 1568F, K591E, S611N, S611I, S611G, S614G, G617E, G617V, T620A, F621V, S636Y, V637M, V637L, V637A, V638G, P639A, P639, Y640N, K642E, Q644P, Q644del, N647D, E652K, Y657C, Y657N, Y657S, M660T, M663S, 1665N, S668F, S668Y, E690_P699del, Y705H, L706P, L706M, T708S, T708N, K709E, F710C, I711T, V713M, V713L, T7141, T714A, and c.2144+1G>A.

Further included within the scope of the present invention are engineered hematopoietic stem cells (HSCs) or T-cells as detailed above. Particularly, the present invention provides a hematopoietic stem cell (HSC) or T-cell, which comprises an exogenous polynucleotide sequence comprising at least a partial or complete sequence of a functional STAT3 gene, said exogenous polynucleotide sequence being integrated in an endogenous STAT3 gene comprising at least one mutation causing Hyper-IgE syndrome (HIES), resulting in the expression of a functional STAT3 polypeptide. More specifically, the present invention provides a hematopoietic stem cell (HSC) or T-cell originating from a patient a originating from a patient suffering from HIES, which comprises an exogenous polynucleotide sequence comprising at least a partial or complete sequence of a functional STAT3 gene, said exogenous polynucleotide sequence being integrated in an endogenous STAT3 gene comprising at least one mutation causing Hyper-IgE syndrome (HIES), resulting in the expression of a functional STAT3 polypeptide.

It is understood that all details given above with respect to the population of cells, especially the details on the exogenous polynucleotide sequence and the type of cells, also apply to the engineered hematopoietic stem cells (HSCs) and T-cells of the present invention.

The present invention further provides engineered HSCs or T-cells cells as well as populations of engineered HSCs or T-cells obtainable by any of the production methods disclosed herein.

### Means and Method for Gene Editing According to the Invention

The present invention further provides means and methods for genetically modifying HSCs or T-cells involving gene editing reagents that specifically target an endogenous STAT3 gene comprising at least one mutation causing Hyper-IgE syndrome (HIES), thereby allowing the restoration of the normal cellular phenotype. Targeted (i.e. site-directed) integration to achieve gene function is suitably done by using sequence-specific reagents inducing DNA cleavage, such as a rare-cutting endonuclease or nickase, and exogenous polynucleotide donor templates bearing homology to the target site and comprising the corrective sequence. Targeted integration can also been achieved using sequence-specific reagents inducing transposition such as described by Owns et al. [15], Voigt et al. or Bhatt and Chalmers [17].

The present invention thus provides sequence-specific reagents inducing DNA cleavage specifically targeting an endogenous STAT3 gene comprising at least one mutation causing Hyper-IgE syndrome (HIES), and polynucleotide donor templates bearing homology to the target site and comprising the corrective sequence, which is preferably optimized.

Accordingly, the present invention provides a polynucleotide donor template characterized in that it comprises at least a partial or complete sequence of a functional STAT3 gene. Such polynucleotide donor template, which is exogenous to the cells, can be provided under different forms, either as double or single stranded polynucleotides that can be electroporated into the cells, such as single-stranded DNAs (ssDNAs), or as part of a viral vector, preferably an AAV vector, through viral transduction. These polynucleotide donor templates are introduced into the cells by methods well known in the art.

In the present invention, single-stranded DNAs or double-stranded DNAs can be advantageous over viral vectors in several respects. For example, single-stranded DNAs or double-stranded DNAs do not contain vector-specific sequences such as LTR or ITR. They may also avoid contamination by undesired plasmid sequences during production process. Furthermore, ssDNAs and dsDNAs may be cheaper to produce under good manufacturing practices (GMP) than viral vectors which are produced in host cells. According to some embodiments, said single-stranded DNA (“ssDNA”) or double-stranded DNAs (dsDNAs) can comprise protection of DNA ends or specific structures (such as hairpin, loop) that will protect the donor template from degradation. They can also comprise modifications such as modified sugar moiety or modified inter-nucleoside linkage as described in WO2012065143. In some embodiments, dsDNA ends can also be covalently closed. The sequences of the ssDNAs and dsDNAs can be optimized more easily as they are usually synthetized in vitro, thereby allowing the optional incorporation of modified bases (e.g., methylation, biotinylation . . . ). In some embodiments, the sequences of the ssDNAs and dsDNAs can incorporate the sequence of a site-specific nuclease such as one described in the present invention. In terms of specificity, ssDNAs are regarded as allowing more specific and stable genomic integration as resorting mainly to rad51 independent mechanism rather than classic homologous recombination (rad51 dependent). Such integration can be further promoted by treating the cells with specific molecules, such as inhibitors of 53BP1 and/or GSE56; and/or siRNA such .rad51siRNA,; and/or Rad59mRNA or helicases mRNAs such as Srs2, UvrD, PcrA, Rep or FBH1.

According to some embodiments, said polynucleotide donor template comprises at least one exon selected from Exons 8 to 24 of STAT3 encoding the amino acid sequence of SEQ ID NOs: 2 to 18, respectively.

According to some embodiments, said polynucleotide donor template comprises at least one exon selected from Exons 8 to 22 of STAT3 encoding the amino acid sequence of SEQ ID NOs: 2 to 16, respectively.

According to some embodiments, said polynucleotide donor template comprises at least Exons 8 to 22 of STAT3 encoding the amino acid sequence of SEQ ID NOs: 2 to 16, respectively.

According to some embodiments, said polynucleotide donor template comprises at least Exons 9 to 22 of STAT3 encoding the amino acid sequence of SEQ ID NOs: 3 to 16, respectively.

According to some embodiments, said polynucleotide donor template comprises at least Exons 10 to 22 of STAT3 encoding the amino acid sequence of SEQ ID NOs: 4 to 16, respectively.

According to some embodiments, said polynucleotide donor template further comprises Exon 23 of STAT3 encoding the amino acid sequence of SEQ ID NO: 17, optionally further comprising Exon 24 of STAT3 encoding the amino acid sequence of SEQ ID NO: 18.

According to some embodiments, said polynucleotide donor template comprises Intron 22 of STAT3 according to SEQ ID NO: 27, which is located upstream of Exon 23 and enables an alternative splicing to Exon 23.

According to some embodiments, said polynucleotide donor template comprises in consecutive order (i.e. from 5′ to 3′), at least Exons 8 to 22 of STAT3, Intron 22 of STAT3 and Exons 23 to 24 of STAT3, wherein Exons 8 to 24 encode the amino acid sequences of SEQ ID NOs: 2 to 18, respectively.

According to some embodiments, said polynucleotide donor template comprises in consecutive order (i.e. from 5′ to 3′) at least Exons 9 to 22 of STAT3, Intron 22 of STAT3 and Exons 23 to 24 of STAT3, wherein Exons 9 to 24 encode the amino acid sequences of SEQ ID NOs: 3 to 18, respectively.

According to some embodiments, said polynucleotide donor template comprises in consecutive order (i.e. from 5′ to 3′) at least Exons 10 to 22 of STAT3, Intron 22 of STAT3 and Exons 23 to 24 of STAT3, wherein Exons 10 to 24 encode the amino acid sequences of SEQ ID NOs: 4 to 18, respectively.

Generally, once integrated in an endogenous STAT3 gene, said polynucleotide donor template allows the expression of a functional STAT3 polypeptide, preferably of SEQ ID NO: 1, and more specifically the expression of the two STAT3 isoforms STAT3alpha and STAT3beta, by said engineered cells.

In order to improve the expression of the functional STAT3 polypeptide and to avoid homologous recombination, said partial or complete sequence of a functional STAT3 gene comprised by said polynucleotide donor template may be codon optimized. Thus, according to some embodiments, said partial or complete sequence of a functional STAT3 gene is codon optimized.

The polynucleotide donor template may comprise upstream of the partial or complete sequence of functional STAT3 a natural or artificial splice site. Such splice site has the advantage of allowing easier splicing and/or better transcription/translation of downstream sequences. Thus, according to some embodiments, said polynucleotide donor template comprises upstream of the partial or complete sequence of functional STAT3 a natural or artificial splice site. According to some embodiments, said polynucleotide donor template comprises upstream of the partial or complete sequence of functional STAT3 an artificial splice site, such as such as the artificial splice site set forth in SEQ ID NO: 28 or SEQ ID NO:29. In order to facilitate targeted (i.e. site-directed) integration in an endogenous STAT3 gene via homologous recombination, the polynucleotide donor template may further comprise left and/or right homology sequences having at least 80% sequence identity with a part of the endogenous STAT3 gene, and more specifically with the target sequence. The polynucleotide donor template may comprise a left homology sequence located 5′ to the partial or complete sequence of a functional STAT3 gene and/or a right homology sequence located 3′ to the partial or complete sequence of a functional STAT3 gene.

Preferred, target sites for site-directed integration are Introns 7, 8 or 9 of the endogenous STAT3 gene. Thus, according to some embodiments, the polynucleotide donor template comprises left and/or right homology sequences having at least 80% sequence identity with SEQ ID NO: 30, 31 or 32. For site-directed integration into Intron 7, the polynucleotide donor template may comprise a left homology sequence having at least 80% sequence identity with SEQ ID NO: 30 located 5′ to the partial or complete sequence of a functional STAT3 gene and/or a right homology sequence having at least 80% sequence identity with SEQ ID NO: 30 located 3′ to the partial or complete sequence of a functional STAT3 gene. For site-directed integration into Intron 8, the polynucleotide donor template may comprise a left homology sequence having at least 80% sequence identity with SEQ ID NO: 31 located 5′ to the partial or complete sequence of a functional STAT3 gene and/or a right homology sequence having at least 80% sequence identity with SEQ ID NO: 31 located 3′ to the partial or complete sequence of a functional STAT3 gene. For site-directed integration into Intron 9, the polynucleotide donor template may comprise a left homology sequence having at least 80% sequence identity with SEQ ID NO: 32 located 5′ to the partial or complete sequence of a functional STAT3 gene and/or a right homology sequence having at least 80% sequence identity with SEQ ID NO: 32 located 3′ to the partial or complete sequence of a functional STAT3 gene.

The polynucleotide donor template may not only comprise sequences homologous to a STAT3 intron of interest, but may also comprise sequences homologous to the adjacent exon(s). By way of example, said left homology sequence may comprise part of Exon 7 and a part of intron 7, while the right homology sequence may comprise part of Intron 7 and part of Exon 8.

According to some embodiments, the polynucleotide donor template comprises a polynucleotide sequence having at least 70%, such as at least 80%, at least 85%, at least 90% or at least 95%, sequence identity with SEQ ID NO: 33, SEQ ID NO:34, SEQ ID NO:35, SEQ ID NO:36, SEQ ID NO:37, SEQ ID NO:41, SEQ ID NO:42, SEQ ID NO:43, SEQ ID NO:44, SEQ ID NO:45 and SEQ ID NO:47. These polynucleotide sequences which are generally ssDNAs or comprised into an AAV vector are generally codon optimized and therefore easily detectable by PCR or hybridization tools.

Preferred optimized versions of the polynucleotide donor template according to the present invention are SEQ ID NO:41, SEQ ID NO:42, SEQ ID NO:43, SEQ ID NO:44, SEQ ID NO:45 and SEQ ID NO:47. Best expression results were obtained by using polynucleotide donor templates comprising SEQ ID NO:45, especially in conjunction with cleavage with a rare cutting endonuclease into the target sequence SEQ ID NO:38, especially a TALE-nuclease heterodimer comprising at least one polypeptides of SEQ ID NO:21 or SEQ ID NO:22, or a TALE-nuclease monomer cleaving SEQ ID NO:38 or at least 90%, preferably 95% identity with SEQ ID NO:21 or SEQ ID NO:22.

In order to facilitate targeted (i.e. site-directed) integration in an endogenous STAT3 gene via NHEJ repair mechanism at the cleaved locus, the polynucleotide donor template may comprise microhomologies, i.e. short homologous DNA sequences.

Further, in order to facilitate the nuclear export, translation and stability of mRNA, the polynucleotide donor template may further comprise a polyadenylation sequence. Non-limiting examples of a polyadenylation sequence include polyadenylation sequences from SV40, hGH (human Growth Hormone), bGH (bovine Growth Hormone), or rbGlob (rabbit beta-globin). According to some embodiments, the polyadenylation sequence comprises a polynucleotide sequence having at least 70%, such as at least 80%, at least 85%, at least 90% or at least 95%, sequence identity with SEQ ID NO: 46.

The present invention further provides a vector, such as plasmid, PCR product, viral vector, and more specifically a non-integrative viral vector such as an AAV or IDLV vector, comprising a polynucleotide donor template of the present invention. Thus, according to some embodiments, the vector is an AAV vector, preferably an AAV6 vector. AAV vectors, and especially AAV6, are particularly suited for transduction of the polynucleotide donor template into cells and to perform integration by homologous recombination directed by rare-cutting endonucleases as described for instance by Sather, B. D. et al. [18].

The present invention further provides the ex vivo use of the polynucleotide donor template according to the invention or the vector of the invention comprising same in gene editing HSCs or T-cells, notably HSCs or T-cells of a patient suffering from HIES.

The present invention further provides sequence-specific reagents inducing DNA cleavage that are capable of targeting and cleaving a sequence within an endogenous STAT3 gene, and more specifically within an endogenous STAT3 gene of a patient suffering from HIES. According to some embodiments, the sequence-specific reagents inducing DNA cleavage is capable of cleaving a sequence within the STAT3 gene, preferably comprised within the intron 7 polynucleotide sequence (SEQ ID NO: 30), intron 8 polynucleotide sequence (SEQ ID NO: 31) or intron 9 polynucleotide sequence (SEQ ID NO: 32).

Non-limiting examples of a “sequence-specific reagent inducing DNA cleavage” according to the invention include reagents that have nickase or endonuclease activity. The sequence-specific reagent can be a chimeric polypeptide comprising a DNA binding domain and another domain displaying catalytic activity. Such catalytic activity can be for instance a nuclease to perform gene inactivation, or nickase or double nickase to preferentially perform gene insertion by creating cohesive ends to facilitate gene integration by homologous recombination, or to perform base editing as described in Komor et al. [19].

In general, the sequence specific reagents of the present invention have the ability to recognize and bind a “target sequence” within the endogenous STAT3 gene, notably a “target sequence” within an intron of the endogenous STAT3 gene, such as Intron 7 (SEQ ID NO: 30), Intron 8 (SEQ ID NO: 31) or Intron 9 (SEQ ID NO: 32). The “target sequence” which is recognized and bound by the sequence specific reagents is usually selected to be rare or unique in the cell's genome, and more extensively in the human genome, as can be determined using software and data available from human genome databases, such as http://www.ensembl.org/index.html. Such “target sequences” are preferably spanned by those having identity with SEQ ID NO. 30, SEQ ID NO. 31 or SEQ ID NO. 32.

According to some embodiments, the sequence-specific reagent inducing DNA cleavage is a rare-cutting endonuclease. “Rare-cutting endonucleases” are sequence-specific endonuclease reagents of choice, insofar as their recognition sequences generally range from 10 to 50 successive base pairs, preferably from 12 to 30 bp, and more preferably from 14 to 20 bp.

According to some embodiments, said rare-cutting endonuclease is an “engineered” or “programmable” rare-cutting endonuclease, such as a homing endonuclease as described for instance by Arnould S., et al. (WO2004067736), a zing finger nuclease (ZFN) as described, for instance, by Urnov F., et al. [20], a TALE-nuclease as described, for instance, by Mussolino et al. [21], or a MegaTAL nuclease as described, for instance by Boissel et al. [22].

Due to their higher specificity, TALE-nuclease have proven to be particularly appropriate sequence specific nuclease reagents for therapeutic applications, especially under heterodimeric forms—i.e. working by pairs with a “right” monomer (also referred to as “5” or “forward”) and ‘left” monomer (also referred to as “3” or “reverse”) as reported for instance by Mussolino et al. [23]. Thus, according to some embodiments, said rare-cutting endonuclease is a TALE-nuclease.

According to some embodiments, said heterodimeric TALE-nuclease comprises a monomer targeting a STAT3 polynucleotide sequence selected from, SEQ ID NO:38, SEQ ID NO:39 and SEQ ID NO:40. According to some embodiments, said TALE-nuclease monomer has at least 80% sequence identity with the polypeptide sequence of any one of SEQ ID NOs: 21 to 26.

According to some embodiments, said TALE-nuclease comprises a first monomer having at least 80%, such as at least 85%, at least 90% or at least 95%, sequence identity with the polypeptide sequence of SEQ ID NO: 21 and second monomer having at least 80%, such as at least 85%, at least 90% or at least 95%, sequence identity with the polypeptide sequence of SEQ ID NO: 22.

According to some embodiments, said TALE-nuclease comprises a first monomer having at least 80%, such as at least 85%, at least 90% or at least 95%, sequence identity with the polypeptide sequence of SEQ ID NO: 23 and second monomer having at least 80%, such as at least 85%, at least 90% or at least 95%, sequence identity with the polypeptide sequence of SEQ ID NO: 24.

According to some embodiments, said TALE-nuclease comprises or a first monomer having at least 80%, such as at least 85%, at least 90% or at least 95%, sequence identity with the polypeptide sequence of SEQ ID NO: 25 and second monomer having at least 80%, such as at least 85%, at least 90% or at least 95%, sequence identity with the polypeptide sequence of SEQ ID NO: 26.

According to some embodiment, the rare-cutting endonuclease is an RNA guided endonuclease, such as Cas9 or Cpf1, to be used in conjunction with a RNA-guide as per, inter alia, the teaching by Doudna, J. et al., and Zetsche, B. et al. [25]. The RNA-guide is design such to hybridize a target sequence.

According to some embodiments, the sequence-specific reagent inducing DNA cleavage is a nickase, such as described in EP3004349.

The present invention further provides a polynucleotide encoding the sequence-specific reagent inducing DNA cleavage of the present invention.

The present invention further provides a vector, such as a viral vector, and more specifically a non-integrative viral vector such as an AAV or IDLV vector, comprising a polynucleotide encoding the sequence-specific reagent inducing DNA cleavage of the present invention.

The present invention further provides the ex vivo use of the sequence specific reagent inducing DNA cleavage of the invention, the polynucleotide of the invention encoding same or the vector of the invention comprising said polynucleotide in gene editing HSCs or T-cells, notably HSCs or T-cells of a patient suffering from HIES.

The present invention further provides a method for engineering a population of T-cells or HSCs comprising the steps of:

Introducing into T-cells or HSCs originating from a patient suffering from HIES a polynucleotide donor template comprising at least a partial or complete sequence of a functional STAT3 gene, such as the polynucleotide donor template according to the invention;

Introducing into said T-cells or HSCs a sequence-specific reagent inducing DNA cleavage to obtain cleavage of the endogenous STAT3 gene in an intron sequence, preferably in Intron 7, 8 or 9, and inserting at this locus said polynucleotide donor template by homologous recombination or NHEJ; and

Optionally, cultivating the cells for expression of STAT3alpha and STAT3beta isoforms.

The method has been particularly designed to obtain engineered HSCs or T-cells for the treatment of a patient suffering from HIES by gene therapy, more particularly by integrating corrected polynucleotide sequences at the endogenous STAT3 locus using the polynucleotide donor template and the sequence-specific reagent inducing DNA cleavage described herein.

The method is preferably practiced ex vivo to obtain stably engineered HSCs or T-cells. The resulting engineered HSCs or T-cells can be then engrafted into a patient in need thereof for a long term in-vivo production of engineered cells that will comprise said polynucleotide donor template, respectively said exogenous polynucleotide sequence as detailed herein.

According to some embodiments, said polynucleotide donor template is introduced into said T-cells or HSCs via a vector of the present invention.

According to some embodiments, said sequence-specific reagent inducing DNA cleavage is a sequence-specific reagent inducing double strand break of genomic DNA.

According to some embodiments, the sequence-specific reagent inducing DNA cleavage, such as a rare-cutting endonuclease, is transiently expressed or delivered in the cells, meaning that said reagent is not supposed to integrate into the genome or persist over a long period of time, such as be the case of RNA, more particularly mRNA, proteins or complexes mixing proteins and nucleic acids (e.g.: Ribonucleoproteins). In general, 80% of the sequence-specific reagent is degraded within 30 hours, preferably by 24, more preferably by 20 hours after transfection. Preferably, the sequence-specific reagent inducing DNA cleavage, such as a rare-cutting endonuclease, is introduced into the cell in the form of a nucleic acid molecule, such as under DNA or RNA molecule, preferably mRNA molecule, encoding said sequence-specific reagent, and will be expressed by the transfected cell. A sequence-specific reagent inducing DNA cleavage, such as a rare-cutting endonuclease, under mRNA form is preferably synthetized with a cap to enhance its stability according to techniques well known in the art, as described, for instance, by Kore A. L., et al. (Locked nucleic acid (LNA)-modified dinucleotide mRNA cap analogue: synthesis, enzymatic incorporation, and utilization (2009) J Am Chem Soc. 131(18):6364-5).

According to some embodiments, said sequence-specific reagent inducing DNA cleavage is introduced into said T-cells or HSCs via a vector of the present invention.

According to some embodiments, said sequence-specific reagent inducing DNA cleavage is introduced into said T-cells or HSCs as mRNA by electroporation.

According to some embodiments, said sequence-specific reagent inducing DNA cleavage is introduced into said T-cells or HSCs via nanoparticles, preferably nanoparticles which are coated with ligands, such as antibodies, having a specific affinity towards a HSC surface protein, such as CD105 (Uniprot #P17813), or a T-cell surface protein. Preferred nanoparticles are biodegradable polymeric nanoparticles in which the sequence specific reagent under polynucleotide form is complexed with a polymer of polybeta amino ester and coated with polyglutamic acid (PGA).

According to some embodiments, methods of non-viral delivery of the polynucleotide donor template and/or the sequence-specific reagent inducing DNA cleavage can be used such as electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery.

According to some embodiments, electroporation steps can be used to transfect cells. In general, electroporation steps that are used to transfect cells are typically performed in closed chambers comprising parallel plate electrodes producing a pulse electric field between said parallel plate electrodes greater than 100 volts/cm and less than 5,000 volts/cm, substantially uniform throughout the treatment volume such as described in WO/2004/083379, especially from page 23, line 25 to page 29, line 11. One such electroporation chamber preferably has a geometric factor (cm-1) defined by the quotient of the electrode gap squared (cm2) divided by the chamber volume (cm3), wherein the geometric factor is less than or equal to 0.1 cm-1, wherein the suspension of the cells and the sequence-specific reagent is in a medium which is adjusted such that the medium has conductivity in a range spanning 0.01 to 1.0 milliSiemens. In general, the suspension of cells undergoes one or more pulsed electric fields. With the method, the treatment volume of the suspension is scalable, and the time of treatment of the cells in the chamber is substantially uniform.

### Activation and Gene Editing of HSCs

Peripheral blood cells are preferably used from mobilised peripheral blood (MPB) leukapheresis, CD34+ cells are generally processed and enriched using immunomagnetic beads such as CliniMACS, Purified CD34+ cells are seeded on culture bags at 1×10⁶ cells/ml in serum-free medium in the presence of cell culture grade Stem Cell Factor (SCF), preferably 300 ng/ml, preferably with FMS-like tyrosine kinase 3 ligand (FLT3L) 300 ng/ml, and Thrombopoietin (TPO), preferably around 100 ng/ml and further interleukline IL-3, preferably more than 60 ng/ml (all from CellGenix) during between preferably 12 and 24 hours before being transferred to an electroporation buffer comprising mRNA encoding the sequence specific reagent. Upon electroporation, the cells are preferably cryopreserved.

### Activation and Expansion of T Cells

Whether prior to or after genetic modification, the T-cells according to the present invention can be activated or expanded, even if they can activate or proliferate independently of antigen binding mechanisms. T-cells, in particular, can be activated and expanded using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo. T cells are generally expanded by contact with an agent that stimulates a CD3 TCR complex and a co-stimulatory molecule on the surface of the T cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T-cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, 1L-4, 1L-7, GM-CSF, -10, -2, 1L-15, TGFp, and TNF- or any other additives for the growth of cells known to the skilled artisan. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02). T cells that have been exposed to varied stimulation times may exhibit different characteristics

In another particular embodiment, said cells can be expanded by co-culturing with tissue or cells. Said cells can also be expanded in vivo, for example in the patient's blood after administrating said cell into the patient.

### Therapeutic Methods, Compositions and Uses of the Invention

The present invention described above allows producing engineered HSCs or T-cells or populations comprising such engineered cells in which the initially defective endogenous STAT3 gene sequence causing Hyper-IgE syndrome (HIES) has been replaced by a corrective STAT3 sequence, thereby restoring the normal cellular phenotype by enabling alternative splicing and hence expression of both STAT3 isoforms. This makes the engineered HSCs or T-cells, respectively the population of HSCs or T-cells of the present invention particularly useful in the treatment of HIES. These cells or population of cells may also be used in the manufacture of a medicament, such as a medicament for use in the treatment of Hyper-IgE syndrome (HIES).

The present invention thus provides the engineered HSCs or T-cells, respectively the population of HSCs or T-cells of the present invention for use in the treatment of Hyper-IgE syndrome (HIES).

The present invention thus provides the engineered HSCs, respectively the population of HSCs of the present invention for use in stem cell transplantation, such as bone marrow transplantation.

The present invention also provides a pharmaceutical composition comprising at least one engineered HSC or T-cell of the present invention, or a population of engineered hematopoietic stem cells (HSCs) or T-cells of the present invention, and a pharmaceutically acceptable excipient and/or carrier.

Suitable, such composition comprises the engineered HSC or T-cell, or the population of engineered hematopoietic stem cells (HSCs) or T-cells, in a therapeutically effective amount. An “effective amount” or “therapeutically effective amount” refers to that amount of a composition described herein which, when administered to a subject, is sufficient to provides a therapeutic or prophylactic benefit, i.e. aids in treating or preventing the disease. The amount of a composition that constitutes a “therapeutically effective amount” may vary depending on the cell preparations, the condition and its severity, the manner of administration, and the age of the subject to be treated, but can be determined routinely by one of ordinary skill in the art having regard to his own knowledge and to this disclosure.

The invention is thus more particularly drawn to a therapeutically effective population of engineered HSCs or T-cells, wherein at least 30%, preferably 50%, more preferably 80% of the cells in said population have been modified according to any one the methods described herein. Said therapeutically effective population of engineered HSCs or T-cells, as per the present invention, comprises cells with a corrected endogenous STAT3 locus, allowing the expression of both STAT3 isoforms.

Suitable pharmaceutically acceptable excipients and carriers are well-known to the skilled person, and have been described in the literature, such as in Remington's Pharmaceutical Sciences, the Handbook of Pharmaceutical Additives or the Handbook of Pharmaceutical Excipients.

In some embodiments, the invention provides a cryopreserved pharmaceutical composition comprising: (a) a viable composition of engineered HSCs or T-cells (b) an amount of cryopreservative sufficient for the cryopreservation of the HSC or T-cells; and (c) a pharmaceutically acceptable carrier.

As used herein, “cryopreservation” refers to the preservation of cells by cooling to low sub-zero temperatures, such as (typically) 77 K or −196° C. (the boiling point of liquid nitrogen). At these low temperatures, any biological activity, including the biochemical reactions that would lead to cell death, is effectively stopped. Cryoprotective agents are often used at sub-zero temperatures to preserve the cells from damage due to freezing at low temperatures or warming to room temperature. The injurious effects associated with freezing can be circumvented by (a) use of a cryoprotective agent, (b) control of the freezing rate, and (c) storage at a temperature sufficiently low to minimize degradative reactions. Cryoprotective agents which can be used include but are not limited to dimethyl sulfoxide (DMSO), glycerol, polyvinylpyrrolidine, polyethylene glycol, albumin, dextran, sucrose, ethylene glycol, i-erythritol, D-Sorbitol, D-mannitol, D-sorbitol, i-inositol, D-lactose, choline chloride, amino acids, methanol, acetamide, glycerol monoacetate, and inorganic salts. In a preferred embodiment, DMSO is used, a liquid which is nontoxic to cells in low concentration. Being a small molecule, DMSO freely permeates the cell and protects intracellular organelles by combining with water to modify its freezability and prevent damage from ice formation. Addition of plasma (e.g., to a concentration of 20-25%) can augment the protective effect of DMSO. After the addition of DMSO, cells should be kept at 0-4° C. until freezing, since DMSO concentrations of about 1% are toxic at temperatures above 4° C.

The present invention provides the pharmaceutical composition of the present invention for use in the treatment of Hyper-IgE syndrome (HIES).

The present invention also provides the pharmaceutical composition of the present invention for use in stem cell transplantation, such as bone marrow transplantation.

The present invention also provides the polynucleotide donor template according to the invention or the vector of the invention comprising same for use in the treatment of Hyper-IgE syndrome (HIES).

The present invention also provides the sequence specific reagent inducing DNA cleavage of the invention, the polynucleotide of the invention encoding same or the vector of the invention comprising said polynucleotide for use in the treatment of Hyper-IgE syndrome (HIES).

The present invention also provides the sequence specific reagent inducing DNA cleavage of the invention, the polynucleotide of the invention encoding same or the vector of the invention comprising said polynucleotide in combination with the polynucleotide donor template according to the invention or the vector of the invention comprising same for use in the treatment of Hyper-IgE syndrome (HIES).

The present invention also provides the sequence specific reagent inducing DNA cleavage of the invention, the polynucleotide of the invention encoding same or the vector of the invention comprising said polynucleotide in combination with the polynucleotide donor template according to the invention or the vector of the invention comprising same for use in gene editing in vivo HSCs or T-cells, notably HSCs or T-cells of a patient suffering from HIES.

The present invention also provides a method of treating Hyper-IgE syndrome (HIES) in a patient in need thereof, the method comprising administering a therapeutically effective amount of an engineered HSC or T-cell, respectively a population of HSCs or T-cells of the present invention to said patient.

The present invention also provides a method of treating Hyper-IgE syndrome (HIES) in a patient in need thereof, by heterologous expression of at least a partial or complete sequence of a functional STAT3 gene, wherein said heterologous partial or complete STAT3 gene sequence restores functional expression of STAT3alpha and STAT3beta isoforms in T-cells. According to some embodiments, the partial or complete STAT3 gene sequence is comprise by an exogenous polynucleotide sequence as defined herein above.

The present invention also provides a method of treating Hyper-IgE syndrome (HIES) in a patient in need thereof by gene therapy, wherein said gene therapy comprises introducing into HSCs or T-cells at least one corrected STAT3 exon sequence into an endogenous genomic STAT3 sequence by targeted gene integration. According to some embodiments, said HSCs or T-cells originate from the patient. According to some embodiments, said gene therapy comprises introducing into HSCs or T-cells at least one exogenous STAT3 gene sequence as defined herein above.

Generally, the treatment of HIES according to the invention can be ameliorating, curative or prophylactic.

The administration of the cells or population of cells according to the present invention may be carried out in any convenient manner, including injection, transfusion, implantation or transplantation. The cells or population of cells according to the present invention may be administered to a patient by intravenous injection.

The administration of the cells or population of cells can consist of the administration of 10⁴-10⁸ gene edited cells per kg body weight, preferably 10⁵ to 10⁶ cells/kg body weight including all integer values of cell numbers within those ranges. The present invention thus can provide more than 10 doses comprising between 10⁴ to 10⁶ gene edited cells per kg body weight originating from a single patient's sampling.

The cells or population of cells can be administrated in one or more doses. According to some embodiments, the therapeutic effective number of cells is administrated as a single dose, especially when permanent engraftment of the engineered HSCs is sought to definitely cure the disease.

As an alternative to permanent HSCs engraftment to cure HIES, which is a heavy and aggressive treatment for the immune system, the present invention provides with the method of directly engineering T-cells, which are subsequently expanded and frozen for their sequential use. This strategy allows the possibility of multiple re-dosing of the patient over a long period of time, which can span several years. According to some embodiments, the therapeutic effective number of cells is administrated as more than one dose over a period time. Timing of administration is within the judgment of managing physician and depends on the clinical condition of the patient. The dosage administrated will be dependent upon the age, health and weight of the patient receiving the treatment, the kind of concurrent treatment, if any, frequency of treatment and the nature of the effect desired.

### Other Definitions

Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a ort, v represents g, a or c, b represents g, t or c, h represents a, tor c, and n represents g, a, t or c.

“As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.

By “mutation” is intended the substitution, deletion, insertion of up to one, two, three, four, five, six, seven, eight, nine, ten, eleven, twelve, thirteen, fourteen, fifteen, twenty, twenty five, thirty, forty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.

By “gene” is meant the basic unit of heredity, consisting of a segment of DNA arranged in a linear manner along a chromosome, which codes for a specific protein or segment of protein. A gene typically includes a promoter, a 5′ untranslated region, one or more coding sequences (exons), optionally introns, a 3′ untranslated region. The gene may further comprise a terminator, enhancers and/or silencers.

As used herein, the term “locus” is the specific physical location of a DNA sequence (e.g. of a gene, such as the STAT3 gene) in a genome. The term “locus” can refer to the specific physical location of a rare-cutting endonuclease target sequence on a chromosome. Such a locus can comprise a target sequence that is recognized and/or cleaved by a sequence-specific reagent according to the invention. It is understood that the locus of interest of the present invention can not only qualify a nucleic acid sequence that exists in the main body of genetic material (i.e. in a chromosome) of a cell but also a portion of genetic material that can exist independently to said main body of genetic material such as plasmids, episomes, virus, transposons or in organelles such as mitochondria as non-limiting examples.

By “DNA target”, “DNA target sequence”, “target DNA sequence”, “nucleic acid target sequence”, or “target sequence” it is intended a polynucleotide sequence that can be targeted and processed by a sequence-specific reagent according to the present invention. These terms refer to a specific DNA location, preferably a genomic location in a cell, but also a portion of genetic material that can exist independently to the main body of genetic material such as plasmids, episomes, virus, transposons or in organelles such as mitochondria as non-limiting example. The target sequence is defined by the 5′ to 3′ sequence of one strand of said target. Generally, the target sequence is adjacent or in the proximity of the locus to be processed either upstream (5′ location) or downstream (3′ location). In a preferred embodiment, the target sequences and the proteins are designed in order to have said locus to be processed located between two such target sequences. Depending on the catalytic domains of the proteins, the target sequences may be distant from 5 to 50 bases (bp), preferably from 10 to 40 bp, more preferably from 15 to 30, even more preferably from 15 to 25 bp. These later distances define the spacer referred to in the description and the examples. It can also define the distance between the target sequence and the nucleic acid sequence being processed by the catalytic domain on the same molecule.

As used herein, “exogenous” sequence generally refers to any nucleotide or nucleic acid sequence that was not initially present at the selected locus. By opposition “endogenous sequence” means a cell genomic sequence initially present at a locus. An “exogenous” sequence is thus a foreign sequence introduced into the cell, and thus allows distinguishing engineered cells over sister cells that have not integrated this exogenous sequence at the locus.

By “sequence-specific reagent inducing DNA cleavage” it is meant any active molecule that has the ability to specifically recognize a selected polynucleotide sequence in a genomic locus, preferably of at least 9 bp, more preferably of at least 10 bp and even more preferably of at least 12 bp in length, and that catalyzes the breakage of the covalent backbone of a polynucleotide. Non-limiting examples of a “sequence-specific reagent inducing DNA cleavage” according to the invention include reagents that have nickase or endonuclease activity. The sequence-specific reagent can be a chimeric polypeptide comprising a DNA binding domain and another domain displaying catalytic activity. Such catalytic activity can be for instance a nuclease to perform gene inactivation, or nickase or double nickase to preferentially perform gene insertion by creating cohesive ends to facilitate gene integration by homologous recombination, or to perform base editing as described in Komor et al. (2016) Nature 19; 533(7603):420-4.

The term “endonuclease” generally refers to any wild-type or variant enzyme capable of catalyzing the hydrolysis (cleavage) of bonds between nucleic acids within a DNA or RNA molecule, preferably a DNA molecule. Endonucleases do not cleave the DNA or RNA molecule irrespective of its sequence, but recognize and cleave the DNA or RNA molecule at specific polynucleotide sequences, further referred to as “target sequences” or “target sites”. Endonucleases can be classified as rare-cutting endonucleases when having typically a polynucleotide recognition site greater than 10 base pairs (bp) in length, more preferably of 14-55 bp. Rare-cutting endonucleases significantly increase homologous recombination by inducing DNA double-strand breaks (DSBs) at a defined locus thereby allowing gene repair or gene insertion therapies (Pingoud, A. and G. H. Silva (2007). Precision genome surgery. Nat. Biotechnol. 25(7): 743-4.).

The term “cleavage” refers to the breakage of the covalent backbone of a polynucleotide. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA/RNA hybrid cleavage can result in the production of either blunt ends or staggered ends.

By “vector” is meant a nucleic acid molecule capable of transporting another nucleic acid to which it has been linked. A “vector” in the present invention includes, but is not limited to, a viral vector, a plasmid, a PCR product, a RNA vector or a linear or circular DNA or RNA molecule which may consists of a chromosomal, non-chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e.g. adeno-associated viruses, AAV), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

By “hematopoietic stem cells” it is meant multipotent stem cells derived from the bone marrow, such as heatopoietic progenitor cells (HPC), that have the capacity to self-renew and the unique ability to differentiate into all of the different cell types and tissues of the myeloid or lymphoid cell lineages, including but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSC are CD34−. In addition, HSC also refer to long-term repopulating HSC (LT-HSC) and short-term repopulating HSC (ST-HSC). LT-HSC and ST-HSC are differentiated, based on functional potential and on cell surface marker expression. For example, in some embodiments, human HSC are CD34+, CD38−, CD45RA−, CD90+, CD133+ or CD34+, CD38−, CD45RA−, CD90−, CD133−. In addition, ST-HSC are less quiescent (i.e., more active) and more proliferative than LT-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSC can be used in any of the methods described herein. In some embodiments, ST-HSC are useful because they are highly proliferative and thus, can more quickly give rise to differentiated progeny.

By “long term repopulating HSC” or “LT-HSC” it is meant a type of hematopoietic stem cells capable of maintaining self-renewal and multilineage differentiation potential throughout life. Phenotype markers characteristic for LT-HSCs include, but are not limited to, CD34+, CD38−, CD45RA−, CD90+, and CD133+.

By “primary cell” or “primary cells” it is meant cells taken directly from living tissue (e.g. biopsy material or blood sample) and established for growth in vitro for a limited amount of time, meaning that they can undergo a limited number of population doublings. Primary cells are opposed to continuous tumorigenic or artificially immortalized cell lines. Non-limiting examples of such cell lines are CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; Hela cells; HT-1080 cells; HCT-116 cells; Hu-h7 cells; Huvec cells; Molt 4 cells.

By “originating from” it is meant that a cell or cells, such as HSCs or T-cells, have been obtained from a patient suffering from HIES. In general, cells are provided from patients through a variety of methods known in the art, as for instance by leukapheresis techniques as reviewed by Schwartz J. et al. [Guidelines on the use of therapeutic apheresis in clinical practice-evidence-based approach from the Writing Committee of the American Society for Apheresis: the sixth special issue (2013) J. Clin. Apher. 28(3): 145-284]. HSCs and progenitor cells can be taken from bone marrow, and more particularly from the pelvis, at the iliac crest, using a needle or syringe. Alternatively, HSCs may be harvested from the circulating peripheral blood, while blood donors are injected with a HSC mobilizing agent, such as granulocyte-colony stimulating factor (G-CSF) and/or plerixafor, that induces cells to leave the bone marrow and circulate in the blood vessels. HSCs may also be harvested from cord blood. HSCs may also be obtained from induced pluripotent stem (iPS) cells derived from the patient.

“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, at least 85%, at least 90%, at least 95%, at least 98% or at least 99% identity to specific polypeptides described herein and preferably exhibiting substantially the same functions, as well as polynucleotide encoding such polypeptides, are contemplated. Unless otherwise stated, the present invention encompasses polypeptides and polynucleotides that have the same function and share at least 70%, generally at least 80%, more generally at least 85%, preferably at least 90%, more preferably at least 95% and even more preferably at least 97% with those described herein.

The term “subject” or “patient” as used herein means a human, and more specifically a human suffering from HIES.

As used herein, the term “about” means plus or minus 10% of the numerical value of the number with which it is being used.

Where a numerical limit or range is stated herein, the endpoints are included. Also, all values and subranges within a numerical limit or range are specifically included as if explicitly written out.

As used herein, the indefinite articles “a” and “an” mean “at least one” or “one or more” unless the context clearly dictates otherwise.

As used herein, the terms “comprising”, “including”, “having” and grammatical variants thereof are to be taken as specifying the stated features, steps or components but do not preclude the addition of one or more additional features, steps, components or groups thereof.

The above written description of the invention provides a manner and process of making and using it such that any person skilled in this art is enabled to make and use the same, this enablement being provided in particular for the subject matter of the appended claims, which make up a part of the original description.

Having generally described this invention, a further understanding can be obtained by reference to certain specific examples, which are provided herein for purposes of illustration only, and are not intended to limit the scope of the claimed invention.

### EXAMPLES

STAT3 sequencing analyses of Hyper IgE syndrome patients indicated that the vast majority of disease-causing mutations are located within STAT3 coding exons 10-24 (FIG. 1). Based on this finding, a one-therapy-fits-all approach was developed that sought to replace the STAT3 exon 10-24 sequence with a functional one, so restoring STAT3 expression regardless of the mutation.

### Example 1: Materials and Method

### Cell Culture and Activation

Peripheral blood mononuclear cells (PBMCs) of healthy donors (HD) were isolated from leukoreduction system (LRS) chambers that were kindly provided by the Blood Donation Center of the Medical Center-University of Freiburg (donor consent, anonymized). In brief, blood was retrieved from the LRS chamber, washed with phosphate-buffered saline (PBS, life technologies, Cat. No. 50004147) supplemented with 2 mM EDTA (Sigma-Aldrich, Cat. No. 59418C), and subjected to Biocoll Separation Solution (Biochrom GmbH, Cat. No. 50002912) density gradient centrifugation according to the manufacturer's instructions. After removal of the top plasma layer, white blood cells were washed and frozen in CryoStor CS10 (StemCell Technologies, Cat. No. 07930) at a concentration of 5-50×10⁶ cells/ml and stored in liquid nitrogen until further usage.

PBMCs of STAT3 patients were obtained from the Freeze Biobank of the Medical Center-University of Freiburg (donor consent, anonymized, approval of ethics committee). PBMCs were thawed and seeded for 4 h in X-Vivo 15 medium (Lonza, Cat. No. 60121783) supplemented with 5% human AB Serum (Sigma-Aldrich, Cat. No. H4522) and 200 IU/ml of recombinant human interleukin 2 (rhIL-2; Immunotools, Cat. No. 11340027) at a concentration of 2×10⁶ cells/ml.

For activation, cells were counted and reseeded at a density of 1×10⁶ cells/ml for 3 days in the presence of 5 μl/ml CD2/3/28 Immunocult conjugated with antibodies against CD2, CD3, and CD28 beads (Stemcell Technologies, Cat. No. 10970).

Gene Editing Reagents and mRNA Productions:
TALEN and mRNA Production

STAT3-specific TALE-nucleases, TALEN-i7 (SEQ ID NO: 21 and SEQ ID NO: 22), TALEN-i8 (SEQ ID NO: 23 and SEQ ID NO: 24), TALEN-i9 (SEQ ID NO: 25 and SEQ ID NO: 26) encoding mRNAs were produced using the HiScribe T7 ARCA mRNA Kit (NEB, Cat. No. E2065S) following the manufacturer's instructions.

### Exogenous Polynucleotide Template Constructs:

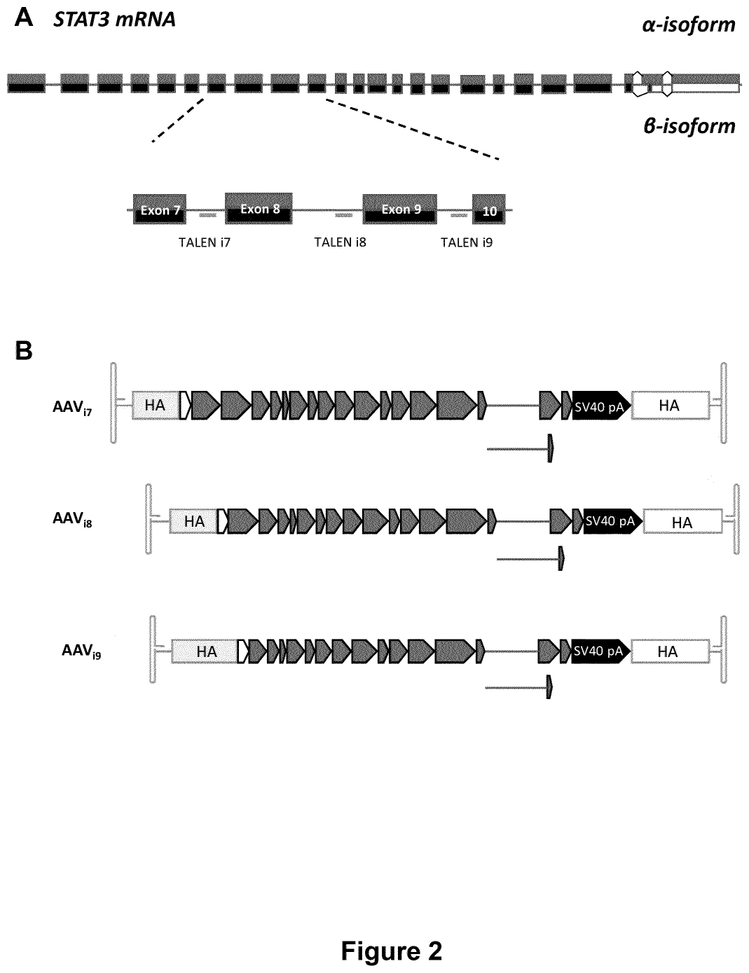

With respect to TALEN-i7 and TALEN-19 cleavage site a specific donor template was created. These vectors contained functional, codon-optimized copies of STAT3 preceded by an artificial splicing site (SEQ ID NO: 28). In all cases, the template sequence was flanked on the left and on the right by homology arms that mediate integration into the respective TALEN-cleaved STAT3 locus (FIG. 2, SEQ ID NO: 41 for TALEN-i7 and SEQ ID NO: 47 for TALEN-i9). Of note, codon-optimization was not extended to the ‘exon 22-intron 22-exon 23′ sequence, which contains the alternative splice donor and acceptor sites that generate the STAT3-α and STAT3-β isoforms. Maintaining the balance between these isoforms are deemed of importance to the therapeutic success and was assessed thoroughly when determining the best TALEN/AAV donor pairing.

Polypeptide and polynucleotide sequences involved in these experiments are detailed in Table 12 at the end of the experimental section.

### Gene Editing Protocols

For gene editing, 1×10⁶ PBMCs per condition were harvested (300×g, 5 min) and the cell pellet resuspended in 50 μl of CliniMACS Electroporation Buffer (Miltenyi Biotec, Cat. No. 170-076-625). The cell suspension was then mixed with TALEN mRNA (1 μg of each TALEN arm), transferred to a 2 mm electroporation cuvette (VWR, Cat. No. 732-1136) and electroporated using a CliniMACS Prodigy device (Miltenyi Biotec). After electroporation, cells were immediately transferred to 400 μl of pre-warmed culture medium. After a recovery of 15 min at 37° C., 1-10×10⁴ genome copies (GC)/cell of the respective AAV6 donor were added and cells cultured at 32° C. overnight. Cells were then transferred to the 37° C. incubator and cells expanded for up to 2 months until final analyses.

### Extraction of Genomic DNA:

Cells were harvested and centrifuged at 300×g for 5 min. After removing the supernatant, genomic DNA was extracted using the NucleoSpin® Tissue Kit (Macherey-Nagel, Cat. No. 740952.250) following the manufacturer's instructions. Genomic DNA concentration was measured with a Nanodrop 2000c spectrophotometer (Thermo Fisher Scientific, USA).

### Assessment of TALEN Cleavage Efficiency by T7E1 Assay;

Mutagenic activity of the respective TALEN was assessed by performing a T7 Endonuclease 1 (17E1) assay as previously published by Dreyer et al. [15]. PCR was carried out using primers and program as indicated in Table 1 and 2, respectively.

TABLE 1
primers used for T7E1 assay

| | | | | Amplicon |
|---|---|---|---|---|
| target | Oligo# | Sequence (5′−>3′) | SEQ ID#: | size |
| i7 (TALEN 3) | 2628 | agtcagtgaccaggcagaag | 48 | 396 bp |
| | 2852 | cagttttctagccgatctaggcag | 49 | |
| i8 (TALEN 5) | 4961 | ctcttcatgcaaggggatgc | 50 | 498 bp |
| | 5029 | ccaccaccactcccggataa | 51 | |
| i9 (TALEN 6) | 3036 | cttctccatctcacctgtatacattcac | 52 | 527 bp |
| | 4206 | agaatgaccctggccaccaac | 53 | |

TABLE 2
PCR program for T7E1 assay
PCR program-TALEN STAT3

| | | |
|---|---|---|
| 98° C. | 2 min | |
| 98° C. | 10 sec | X 30 cycles |
| 67° C. | 20 sec | |
| 72° C. | 15 sec | |
| 72° C. | 3 min | |
| 12° C. | hold | |

### Assessment of TALEN-Induced Indel Frequency by Targeted Amplicon Sequencing

The target sites of the TALENs were amplified by PCR using primers as indicated in Table 3 and Q5 Polymerase (Cat. No. M0493L) on treated PBMCs genomic DNA. Concentration of purified PCR amplicons was determined using the Qubit Fluorometer. Pooled samples (20 ng each) were subjected to DNA library preparation using the NEBNext Ultra II Library Prep Kit for Illumina (NEB, cat. no. #E7645L). DNA libraries were quantified prior to sequencing using the ddPCR Library Quantification Kit for Illumina TruSeq (Bio-Rad, cat. no. #1863040). Sequencing of prepared libraries was performed on an Illumin MiSeq system using the MiSeq Reagent kit v2 (500 cycles) (Illumina, cat. no #MS-102-2003) with paired-end reads at a concentration of 8 pM. After NGS run, reads were analyzed using CRISPResso2 with settings based on the phred33 quality score with minimum average quality score set to 20 (q 20), quantify indels within a window of 40 bp around the predicted cleavage site (w 40) and ignore substitution events for the quantification in order to not consider SNPs.

TABLE 3
Primers use in PCR

| Target | oligonucleotides | Sequence | SEQ ID# | Amplicon size |
|---|---|---|---|---|
| i7 (TALEN 3) | 2628 | agtcagtgaccaggcagaag | 48 | 298 bps |
| | 2629 | tttctgcacgtactccatcg | 54 | |
| i8 (TALEN 5) | 5480 | catatctgggctcagatgcttgtc | 55 | 310 bps |
| | 5029 | ccaccaccactcccggataa | 51 | |
| I9 (TALEN 6) | 5482 | tcaacttcagacccgtcaaca | 56 | 341 bps |
| | 5483 | aactggacgccggtcttgat | 57 | |

### Western Blot Analyses

In order to assess phosphorylation of STAT3, 1×10⁶ HD and STAT3 patient derived PBMCs, respectively, were cultured for 24 h without IL-2 and then either left untreated or stimulated for 30 min with 50 ng/ml of recombinant human IL-6 (rhIL-6, Immunotools, Cat. No. 11340066). Cells were harvested, subjected to protein lysis and run on Western Mini Protean TGX Precast Gels (Gradient: 4-15%, 10-well, 50 μl; BioRad, Cat. No. 456-8084) in accordance with the protocols from the provider. Western blots were performed with the antibodies listed in Table 4.

TABLE 4
list of antibodies used in western blot

| Epitope | Clone | Company/Cat. No. | marker | Dilution |
|---|---|---|---|---|
| human Phospho- | D3A7, | Cell signaling/ | / | 1:2,000 in 5% |
| STAT3 (Y705) | rabbit | 60184682 | | BSA TBS-T |
| human-STAT3 | 124H6, | Cell signaling/ | / | 1:1,000 in 5% |
| | mouse | 9139T | | BSA TBS-T |
| rabbit IgG | DREG- | Cell signaling/ | HRP- | 1:3,000 in 5% |
| | 56 | 60108745 | linked | BSA TBS-T |
| mouse IgG | HI100 | Cell signaling/ | HRP- | 1:3,000 in 5% |
| | | 7076P2 | linked | BSA TBS-T |

ImageJ software (https://imagej.nih.gov/ij/) was used to quantify STAT3 and phospho-STAT3 expression. After background subtraction, each phospho-STAT3 signal was normalized to the respective STAT3 band.

mRNA Extraction and cDNA Synthesis

Up to 5×10⁶ cells were harvested and washed with PBS (300×g, 5 min). The pellets were lysed and mRNA extracted using the RNeasy Mini Kit (Qiagen, Cat. No. 74106) following the manufacturing instructions. Reverse transcription into cDNA was performed using the QuantiTect Reverse Transcription Kit (Qiagen, Cat. No. 205314) according to the manufacturer's instructions.

### Assessment of STAT3 Isoform Expression Using RT-PCR

Amplification of STAT3 isoforms was performed with primers and programs as indicated in Tables 5 and 6 and Taq Polymerase (NEB, Cat. No. M0495S) according to the manufacturer's protocol with 7.5 ng cDNA. The PCR products were analyzed by gel electrophoresis on a 2% agarose gel containing 0.3 μg/ml ethidium bromide in TAE buffer.

TABLE 5
primers used for RT-PCR

| Target | Oligo# | Sequence | SEQ ID# | Amplicon size |
|---|---|---|---|---|
| endogene | 4963 | tgacattcccaaggaggagg | 58 | 248 bps (STAT3α) & |
| | 5037 | caaaggtgagggactcaaactg | 59 | 198 bps (STAT3β) |
| transgene | 5325 | agtactgtcgcccggagtca | 60 | 220 bps (STAT3α) & |
| | 5326 | tcaaacgtaaggctctcaaactgt | 61 | 150 bps (STAT3β) |

TABLE 6
PCR program for RT-PCR
PCR program-STAT3 isoforms

| | | |
|---|---|---|
| 95° C. | 2 min | |
| 95° C. | 20 sec | X 27 cycles |
| 53° C. | 30 sec | |
| 68° C. | 30 sec | |
| 68° C. | 5 min | |
| 12° C. | hold | |

Assessment of Integration Efficiency by ddPCR

Quantification of donor integration was performed by ddPCR using EvaGreen Supermix (Bio-Rad, Cat. No. #1864034) with 25 ng of genomic DNA according to the manufacturer's protocol. In/out-PCRs from both ends were performed with program and primers indicated in Tables 7 and 8, respectively.

TABLE 7
ddPCR program for integration efficiency assessment
PCR program-ddPCR

| | | | |
|---|---|---|---|
| 95° C. | 5 min | | ramp rate 2° C./sec |
| 95° C. | 30 sec | X 40 cycles | ramp rate 2° C./sec |
| 60° C. | 1 min | | ramp rate 2° C./sec |
| 72° C. | 2 min | | ramp rate 2° C./sec |
| 4° C. | 5 min | | ramp rate 2° C./sec |
| 90° C. | 5 min | | ramp rate 2° C./sec |
| 4° C. | hold | | ramp rate 2° C./sec |

TABLE 8
primers used for integration efficiency assessment

| Target | Oligo# | Sequence | SEQ ID# | Amplicon size |
|---|---|---|---|---|
| TALENi7 5′ | 5526 | aaggagcctggtcattaagg | 62 | 970 bps |
| | 5728 | catgattagcaaaagggcctagc | 63 | |
| TALENi7 3′ | 5729 | cctctacaaatgtggtacggcttg | 64 | 470 bps |
| | 5722 | tgatccgcccatcttggtc | 65 | |
| | 5725 | cctgagcctcctgagagattac | 67 | |
| TALENi9 5′ | 5726 | ccttgttcttattgtagtggtctcc | 66 | 399 bps |
| | 5728 | catgattagcaaaagggcctagc | 63 | |
| TALENi9 3′ | 5729 | cctctacaaatgtggtacggcttg | 64 | 695 bps |
| | 5341 | aacacatgctgtgcagctg | 68 | |
| STAT3 Ref. 1 | 5730 | cttgggcaacataacaagacacc | 69 | 520 bps |
| | 5485 | tggctgcagtctgtagaagg | 70 | |
| STAT3 Ref. 2 | 3035 | tggagtctggctgtagccca | 71 | 648 bps |
| | 4025 | gtcctgtttctccttgtcctcagt | 72 | |

### Quantitative Assessment of Isoform Expression

RT-ddPCR was used to quantify expression of the transgene or expression of SOCS3 using EvaGreen Supermix (Bio-Rad, Cat. No. 1864034) according to the manufacturer's protocol with 2-4 ng of cDNA. Thereto, RT-PCR reactions for each target were performed and for normalization to a reaction on HPRT1. The ddPCR program and the primers used are provided in Tables 9 and 10.

TABLE 9
ddPCR program of the RT-ddPCR
PCR program-ddPCR

| | | | |
|---|---|---|---|
| 95° C. | 5 min | | ramp rate 2° C./sec |
| 95° C. | 30 sec | x 40 cycles | ramp rate 2° C./sec |
| 60° C. | 1 min | | ramp rate 2° C./sec |
| 4° C. | 5 min | | ramp rate 2° C./sec |
| 90° C. | 5 min | | ramp rate 2° C./sec |
| 4° C. | hold | | ramp rate 2° C./sec |

TABLE 10
primers used for RT-ddPCR

| | | | SEQ | Amplicon |
|---|---|---|---|---|
| Target | Oligo# | Sequence | ID# | size |
| STAT3α_ | 4963 | TGACATTCCCAAGGAGGAGG | 58 | 136 bps |
| endo | 4965 | ATTGCTGCAGGTCGTTGGTG | 73 | |
| STAT3ß_ | 4963 | TGACATTCCCAAGGAGGAGG | 58 | 140 bps |
| endo | 4964 | TCCAAACTGCATCAATGAATGGTG | 74 | |
| STAT3α_ | 5325 | AGTACTGTCGCCCGGAGTCA | 60 | 107 bps |
| trans | 4965 | ATTGCTGCAGGTCGTTGGTG | 73 | |
| STAT3B_ | 5325 | AGTACTGTCGCCCGGAGTCA | 60 | 111 bps |
| trans | 4964 | TCCAAACTGCATCAATGAATGGTG | 74 | |
| SOCS3 | 4428 | CTTCGATTCGGGACCAGCC | 75 | 99 bps |
| | 4429 | CGGAGCCAGCGTGGATC | 76 | |
| HPRT1 | 4406 | CCCTGGCGTCGTGATTAG | 77 | 104 bps |
| | 4407 | CATGAGGAATAAACACCCTTTCC | 78 | |

### Assessment of Activation and T Cell Subsets

STAT3 patient cells or HD control cells were characterized by staining with anti-CD4, anti-CD25, anti-CD62L and anti-CD45RA antibodies (provided in Table 11). For flow cytometric analyses, 2-5×10⁵ cells were harvested and resuspended in 25 μl of FACS-Buffer consisting of PBS supplemented with 5% FCS (PAN Biotech, Cat. No. P40-47500). Cells were incubated for 30 min at 4° C. in the dark, washed once with FACS buffer (300×g, 5 min) and then resuspended in 150 μl of FACS buffer.

TABLE 11
antibodies used for flow cytometry

| Epitope | Clone | Company/Cat. No. | Dye | Dilution |
|---|---|---|---|---|
| human CD4 | VIT4 | MACS Miltenyi Biotec/ | APC | 1:25 |
| | | 130098158 | | |
| human CD25 | 4E3 | MACS Miltenyi Biotec/ | PE | 1:50 |
| | | 130113282 | | |
| human CD62L | DREG- | BD Biosciences/555543 | FITC | 1:25 |
| | 56 | | | |
| human CD45RA | HI100 | BioLegend/304106 | APC | 1:25 |

Samples were analyzed on a BD Accuri (BD Biosciences) and evaluated with BD Accuri and FlowJo Version 10 software.

### Cytokine Release Assay (CBA)

Supernatants of untreated and edited PBMCs were subjected to a cytometric bead array (CBA, BD Biosciences) to assess IFNγ, TNF, IL-17 and IL-10 release after stimulation. 1×10⁶ cells were cultured in 200 μl of IMDM (Life technologies) supplemented with 10% FCS and 100 IU/ml rhIL-2 in the presence or absence of 10 ng phorbol myristate acetate (PMA, Sigma-Aldrich, Cat. No. P1585) and 200 ng lonomycin (Sigma-Aldrich, Cat. No. 10634). After 5h of incubation, 25 μl of supernatant were harvested, centrifuged (300×g, 5 min) to remove debris and subjected to the CBA according to the manufacturer's instructions with one deviating: the volumes of capture bead and detection antibody per sample was scaled down from 1 μl to 0.3 μl. After final incubation, each sample was resuspended in 300 μl of FACS Buffer and analyzed on a FACS Canto II (BD Biosciences). Data analyses were performed using FlowJo Version 10 by gating on the bead population in the FSC/SSC plot, then identifying the respective capture beads based on the APC/APC-Cy7 pattern, and by then quantifying the cytokine profile of each cytokine based on the mean fluorescence intensity (MFI) in the PE channel.

### Example 2: Identification of the Best TALEN/Template Combination in HD PBMCs

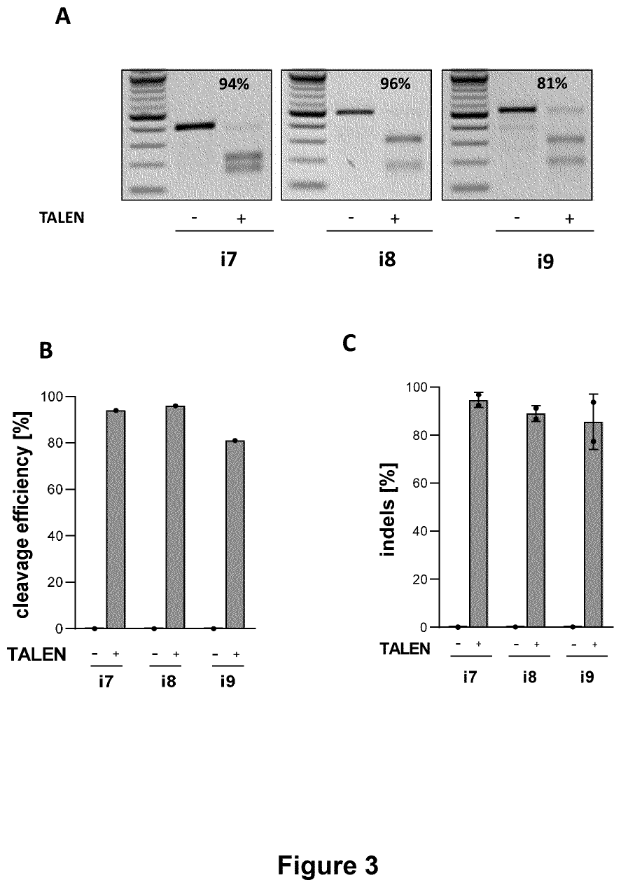

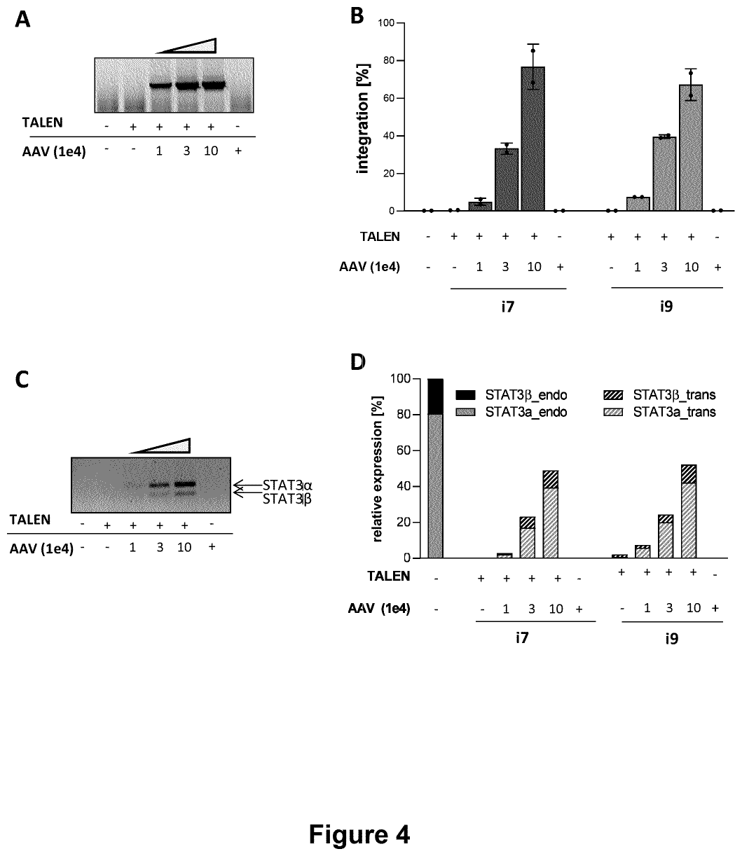

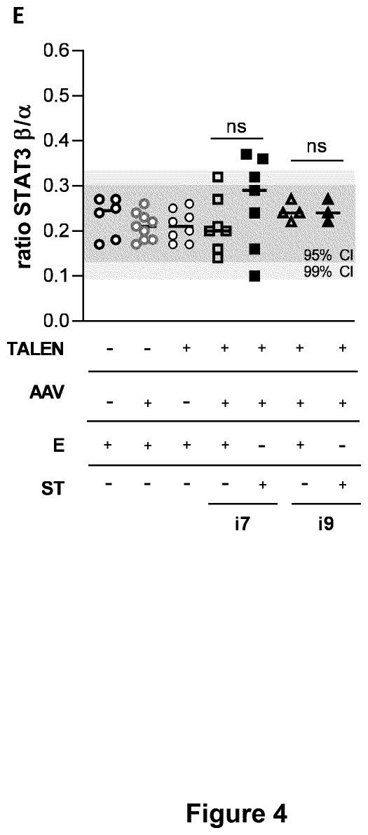

PBMCs of healthy donors (HDs) were first electroporated with the TALEN mRNAs targeting either intron7 (TALEN-i7 SEQ ID NO: 21 and SEQ ID NO: 22), intron8 (TALEN-18 SEQ ID NO: 23 and SEQ ID NO: 24) or intron9 (TALEN-19 SEQ ID NO: 25 and SEQ ID NO: 26). TALEN induced indel frequencies, as determined by T7E1 assay (FIG. 3A, B) and NGS (FIG. 3C), were comparably high for all three TALEN pairs, ranging from 80-95% with both analysis tools. Next, in order to identify the best TALEN/Template combination, percentages of integration and relative expression of the STAT3 isoforms were assessed with TALEN targeting intron7 or intron9 and their respective donor template. To this end, PBMCs were electroporated with the corresponding TALEN mRNAs and then transduced with 1-10×10⁴ GC/cell of the respective AAV6 donor template (FIG. 4). The highest AAV6 donor amount (105 GC/cell) yielded the highest integration frequencies for all tested combinations, ranging between 55-80% (FIG. 4A, B). Relative STAT3 transgene expression was then assessed and comparable level of expression for the two TALEN/AAV6 donor combinations, with about 50% of STAT3 expression stemming from the integrated transgenic STAT3 at the highest AAV6 dose (FIG. 4C, D). Moreover, the STAT3 β/α transgene expression ratios upon genome editing with the two combinations tested were within the correct range (FIG. 4E). TALEN i7 with 105 GC/cell of AAVi7 was selected as the best candidate to go forward.

### Example 3: STAT3 Correction in PBMCs of Three Exemplary STAT3 Patients

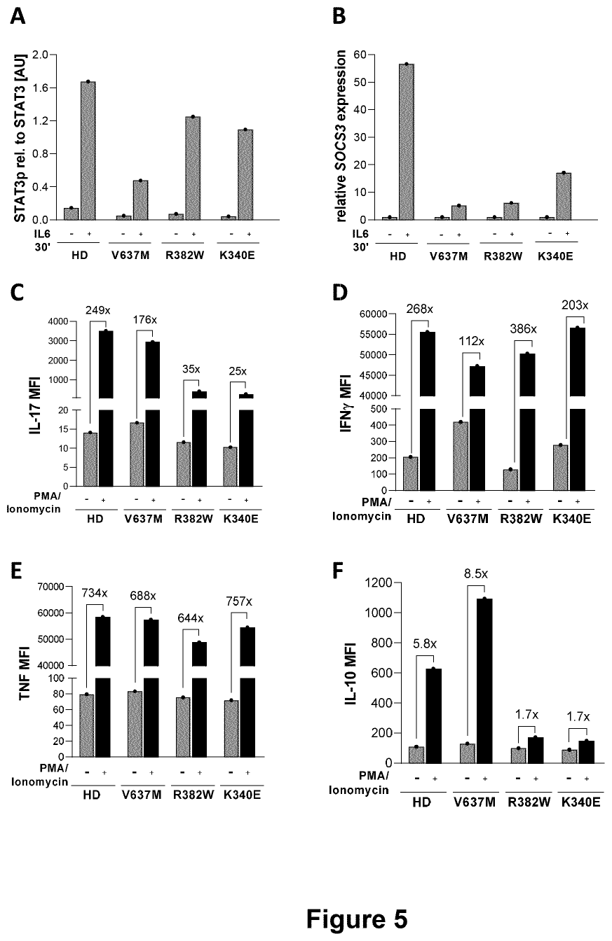

In order to validate, that a TALEN i7/AAVi7-based therapy would be able to serve as a therapy for all Hyper IgE Syndrome patients, independently of the underlying mutation and phenotypical differences, three STAT3 patients (with mutations V637M, R382W and K340E) of the Freiburg cohort were chosen. As expected, the relative STAT3p/STAT3 expression in patient cells that had been stimulated with the STAT3-specific stimulus IL-6, was significantly lower than in HD cells (FIG. 5A). Furthermore, expression of the STAT3 downstream-target SOCS3 was significantly lower (FIG. 5B). On note, Hyper IgE Syndrome patient cells also differed in the cytokine release profile of other hallmark cytokines, such as IL-17, IFNγ, TNF and IL-10 (FIG. 5C-F).

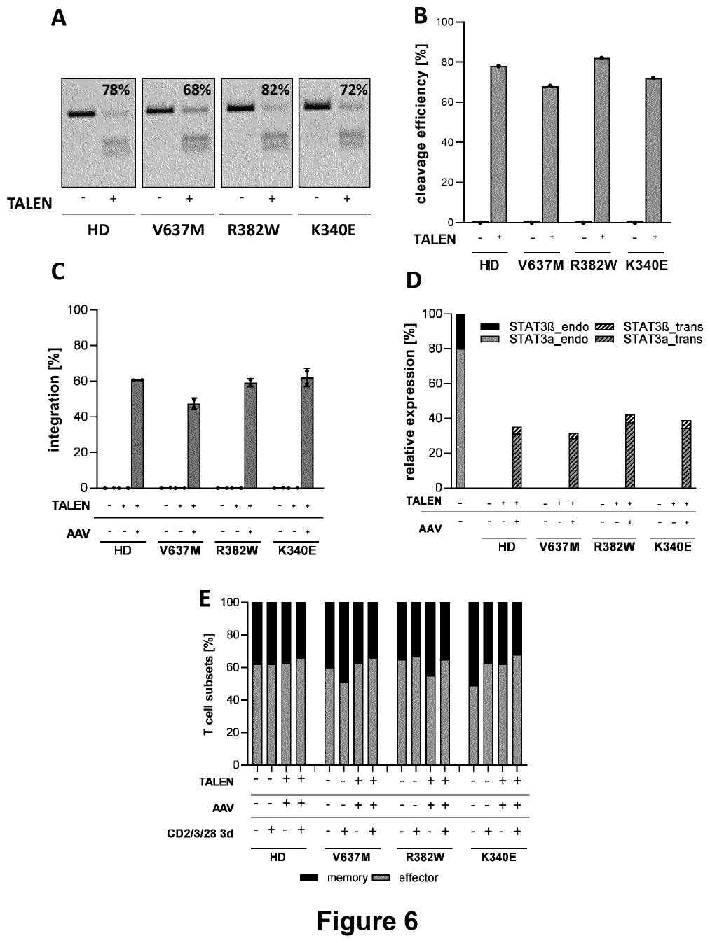

To address this point, cleavage efficiencies after TALEN i7 treatment as well as integration and relative transgene expression after subsequent AAVi7 transduction were assessed. Electroporation of PBMCs with TALEN i7 mRNA resulted in high indel frequencies (68-82%, FIG. 6A, B), integration frequencies (50-60%, FIG. 6C) and STAT3 transgene expression (35-45% of total STAT3, FIG. 6D) which was comparable to HD control. T cell subset analysis after long-term culture revealed a constant memory T cell population (around 40%) in untreated, TALEN i7/AAVi7-edited and restimulated TALEN i7/AAVi7-edited samples (FIG. 6E).

In summary, these findings highlight the suitability of this therapy approach for clinical translation.

TABLE 12
preferred polypeptide and polynucleotide sequences used
in the methods as per the present invention

| SEQ ID NO: # | Name | Polypeptide or polynucleotide sequence |
|---|---|---|
| 21 | TALEN i7 | MGDPKKKRKVIDIADLRTLGYSQQQQEKIKPKVRSTVAQHHEALVGHGFTHA |
| | Left monomer | HIVALSQHPAALGTVAVKYQDMIAALPEATHEAIVGVGKQWSGARALEALLT |
| | | VAGELRGPPLQLDTGQLLKIAKRGGVTAVEAVHAWRNALTGAPLNLTPEQV |
| | | VAIASHDGGKQALETVQRLLPVLCQAHGLTPQQVVAIASNGGGKQALETVQ |
| | | RLLPVLCQAHGLTPEQWVAIASNIGGKQALETVQALLPVLCQAHGLTPEQVV |
| | | AIASNIGGKQALETVQALLPVLCQAHGLTPQQVVAIASNNGGKQALETVQRL |
| | | LPVLCQAHGLTPEQVVAIASNIGGKQALETVQALLPVLCQAHGLTPEQVVAIA |
| | | SNIGGKQALETVQALLPVLCQAHGLTPQQVVAIASNNGGKQALETVQRLLPV |
| | | LCQAHGLTPQQVVAIASNGGGKQALETVQRLLPVLCQAHGLTPQQVVAIAS |
| | | NGGGKQALETVQRLLPVLCQAHGLTPEQWVAIASHDGGKQALETVQRLLPV |
| | | LCQAHGLTPEQVVAIASHDGGKQALETVQRLLPVLCQAHGLTPQQVVAIASN |
| | | GGGKQALETVQRLLPVLCQAHGLTPQQVVAIASNNGGKQALETVQRLLPVL |
| | | CQAHGLTPEQVVAIASHDGGKQALETVQRLLPVLCQAHGLTPQQVVAIASN |
| | | GGGRPALESIVAQLSRPDPALAALTNDHLVALACLGGRPALDAVKKGLGDPI |
| | | SRSQLVKSELEEKKSELRHKLKYVPHEYIELIEIARNSTQDRILEMKVMEFFM |
| | | KVYGYRGKHLGGSRKPDGAIYTVGSPIDYGVIVDTKAYSGGYNLPIGQADEM |
| | | QRYVEENQTRNKHINPNEWWKVYPSSVTEFKFLFVSGHFKGNYKAQLTRLN |
| | | HITNCNGAVLSVEELLIGGEMIKAGTLTLEEVRRKFNNGEINFAAD- |
| 22 | TALEN i7 | MGDPKKKRKVIDIADLRTLGYSQQQQEKIKPKVRSTVAQHHEALVGHGFTHA |
| | right | HIVALSQHPAALGTVAVKYQDMIAALPEATHEAIVGVGKQWSGARALEALLT |
| | monomer | VAGELRGPPLQLDTGQLLKIAKRGGVTAVEAVHAWRNALTGAPLNLTPQQV |
| | | VAIASNNGGKQALETVQRLLPVLCQAHGLTPQQVVAIASNGGGKQALETVQ |
| | | RLLPVLCQAHGLTPEQVVAIASHDGGKQALETVQRLLPVLCQAHGLTPQQV |
| | | VAIASNGGGKQALETVQRLLPVLCQAHGLTPEQVVAIASHDGGKQALETVQ |
| | | RLLPVLCQAHGLTPQQVVAIASNGGGKQALETVQRLLPVLCQAHGLTPEQV |
| | | VAIASNIGGKQALETVQALLPVLCQAHGLTPEQVVAIASNIGGKQALETVQAL |
| | | LPVLCQAHGLTPEQVVAIASHDGGKQALETVQRLLPVLCQAHGLTPEQVVAI |
| | | ASHDGGKQALETVQRLLPVLCQAHGLTPEQVVAIASNIGGKQALETVQALLP |
| | | VLCQAHGLTPEQVVAIASHDGGKQALETVQRLLPVLCQAHGLTPEQVVAIAS |
| | | NIGGKQALETVQALLPVLCQAHGLTPQQVVAIASNGGGKQALETVQRLLPVL |
| | | CQAHGLTPQQVVAIASNGGGKQALETVQRLLPVLCQAHGLTPQQVVAIASN |
| | | GGGRPALESIVAQLSRPDPALAALTNDHLVALACLGGRPALDAVKKGLGDPI |
| | | SRSQLVKSELEEKKSELRHKLKYVPHEYIELIEIARNSTQDRILEMKVMEFFM |
| | | KVYGYRGKHLGGSRKPDGAIYTVGSPIDYGVIVDTKAYSGGYNLPIGQADEM |
| | | QRYVEENQTRNKHINPNEWWKVYPSSVTEFKFLFVSGHFKGNYKAQLTRLN |
| | | HITNCNGAVLSVEELLIGGEMIKAGTLTLEEVRRKFNNGEINFAAD- |
| 23 | TALEN i8b | MGDPKKKRKVIDIADLRTLGYSQQQQEKIKPKVRSTVAQHHEALVGHGFTHA |
| | Left monomer | HIVALSQHPAALGTVAVKYQDMIAALPEATHEAIVGVGKQWSGARALEALLT |
| | | VAGELRGPPLQLDTGQLLKIAKRGGVTAVEAVHAWRNALTGAPLNLTPEQV |
| | | VAIASNIGGKQALETVQALLPVLCQAHGLTPQQVVAIASNNGGKQALETVQR |
| | | LLPVLCQAHGLTPEQVVAIASNIGGKQALETVQALLPVLCQAHGLTPEQVVAI |
| | | ASNIGGKQALETVQALLPVLCQAHGLTPEQVVAIASNIGGKQALETVQALLPV |
| | | LCQAHGLTPQQVVAIASNGGGKQALETVQRLLPVLCQAHGLTPQQVVAIAS |
| | | NGGGKQALETVQRLLPVLCQAHGLTPQQVVAIASNGGGKQALETVQRLLPV |
| | | LCQAHGLTPQQVVAIASNNGGKQALETVQRLLPVLCQAHGLTPEQVVAIASN |
| | | IGGKQALETVQALLPVLCQAHGLTPQQVVAIASNNGGKQALETVQRLLPVLC |
| | | QAHGLTPEQVVAIASNIGGKQALETVQALLPVLCQAHGLTPQQVVAIASNGG |
| | | GKQALETVQRLLPVLCQAHGLTPQQVVAIASNGGGKQALETVQRLLPVLCQ |
| | | AHGLTPQQVVAIASNGGGKQALETVQRLLPVLCQAHGLTPQQVVAIASNGG |
| | | GRPALESIVAQLSRPDPALAALTNDHLVALACLGGRPALDAVKKGLGDPISR |
| | | SQLVKSELEEKKSELRHKLKYVPHEYIELIEIARNSTQDRILEMKVMEFFMKVY |
| | | GYRGKHLGGSRKPDGAIYTVGSPIDYGVIVDTKAYSGGYNLPIGQADEMQR |
| | | YVEENQTRNKHINPNEWWKVYPSSVTEFKFLFVSGHFKGNYKAQLTRLNHIT |
| | | NCNGAVLSVEELLIGGEMIKAGTLTLEEVRRKFNNGEINFAAD-- |
| 24 | TALEN i8b | MGDPKKKRKVIDIADLRTLGYSQQQQEKIKPKVRSTVAQHHEALVGHGFTHA |
| | right | HIVALSQHPAALGTVAVKYQDMIAALPEATHEAIVGVGKQWSGARALEALLT |
| | monomer | VAGELRGPPLQLDTGQLLKIAKRGGVTAVEAVHAWRNALTGAPLNLTPQQV |
| | | VAIASNGGGKQALETVQRLLPVLCQAHGLTPQQVVAIASNGGGKQALETVQ |
| | | RLLPVLCQAHGLTPEQVVAIASNIGGKQALETVQALLPVLCQAHGLTPQQVV |
| | | AIASNGGGKQALETVQRLLPVLCQAHGLTPQQVVAIASNGGGKQALETVQR |
| | | LLPVLCQAHGLTPQQVVAIASNGGGKQALETVQRLLPVLCQAHGLTPQQVV |
| | | AIASNGGGKQALETVQRLLPVLCQAHGLTPEQVVAIASNIGGKQALETVQALL |
| | | PVLCQAHGLTPEQVVAIASHDGGKQALETVQRLLPVLCQAHGLTPEQVVAIA |
| | | SHDGGKQALETVQRLLPVLCQAHGLTPQQVVAIASNGGGKQALETVQRLLP |
| | | VLCQAHGLTPQQVVAIASNGGGKQALETVQRLLPVLCQAHGLTPQQVVAIAS |
| | | NGGGKQALETVQRLLPVLCQAHGLTPQQVVAIASNGGGKQALETVQRLLPV |
| | | LCQAHGLTPEQVVAIASNIGGKQALETVQALLPVLCQAHGLTPQQVVAIASN |
| | | GGGRPALESIVAQLSRPDPALAALTNDHLVALACLGGRPALDAVKKGLGDPI |
| | | SRSQLVKSELEEKKSELRHKLKYVPHEYIELIEIARNSTQDRILEMKVMEFFM |
| | | KVYGYRGKHLGGSRKPDGAIYTVGSPIDYGVIVDTKAYSGGYNLPIGQADEM |
| | | QRYVEENQTRNKHINPNEWWKVYPSSVTEFKFLFVSGHFKGNYKAQLTRLN |
| | | HITNCNGAVLSVEELLIGGEMIKAGTLTLEEVRRKFNNGEINFAAD- |
| 25 | TALEN i9 Left | MGDPKKKRKVIDIADLRTLGYSQQQQEKIKPKVRSTVAQHHEALVGHGFTHA |
| | monomer | HIVALSQHPAALGTVAVKYQDMIAALPEATHEAIVGVGKQWSGARALEALLT |
| | | VAGELRGPPLQLDTGQLLKIAKRGGVTAVEAVHAWRNALTGAPLNLTPQQV |
| | | VAIASNNGGKQALETVQRLLPVLCQAHGLTPEQVVAIASNIGGKQALETVQA |
| | | LLPVLCQAHGLTPEQVVAIASHDGGKQALETVQRLLPVLCQAHGLTPQQVVA |
| | | IASNGGGKQALETVQRLLPVLCQAHGLTPQQVVAIASNGGGKQALETVQRLL |
| | | PVLCQAHGLTPEQVVAIASHDGGKQALETVQRLLPVLCQAHGLTPQQVVAIA |
| | | SNGGGKQALETVQRLLPVLCQAHGLTPQQVVAIASNNGGKQALETVQRLLP |
| | | VLCQAHGLTPQQVVAIASNNGGKQALETVQRLLPVLCQAHGLTPQQVVAIAS |
| | | NGGGKQALETVQRLLPVLCQAHGLTPEQVVAIASHDGGKQALETVQRLLPV |
| | | LCQAHGLTPEQVVAIASNIGGKQALETVQALLPVLCQAHGLTPQQVVAIASN |
| | | GGGKQALETVQRLLPVLCQAHGLTPQQVVAIASNNGGKQALETVQRLLPVL |
| | | CQAHGLTPQQVVAIASNNGGKQALETVQRLLPVLCQAHGLTPQQVVAIASN |
| | | GGGRPALESIVAQLSRPDPALAALTNDHLVALACLGGRPALDAVHHGLGDPI |
| | | SRSQLVKSELEEKKSELRHKLKYVPHEYIELIEIARNSTQDRILEMKVMEFFM |
| | | KVYGYRGKHLGGSRKPDGAIYTVGSPIDYGVIVDTKAYSGGYNLPIGQADEM |
| | | QRYVEENQTRNKHINPNEWWKVYPSSVTEFKFLFVSGHFKGNYKAQLTRLN |
| | | HITNCNGAVLSVEELLIGGEMIKAGTLTLEEVRRKFNNGEINFAAD- |
| 26 | TALEN i9 | MGDPKKKRKVIDIADLRTLGYSQQQQEKIKPKVRSTVAQHHEALVGHGFTHA |
| | right | HIVALSQHPAALGTVAVKYQDMIAALPEATHEAIVGVGKQWSGARALEALLT |
| | monomer | VAGELRGPPLQLDTGQLLKIAKRGGVTAVEAVHAWRNALTGAPLNLTPQQV |
| | | VAIASNNGGKQALETVQRLLPVLCQAHGLTPEQVVAIASNIGGKQALETVQA |
| | | LLPVLCQAHGLTPQQVVAIASNNGGKQALETVQRLLPVLCQAHGLTPQQVV |
| | | AIASNNGGKQALETVQRLLPVLCQAHGLTPEQVVAIASNIGGKQALETVQALL |
| | | PVLCQAHGLTPEQVVAIASNIGGKQALETVQALLPVLCQAHGLTPEQVVAIAS |
| | | NIGGKQALETVQALLPVLCQAHGLTPQQVVAIASNNGGKQALETVQRLLPVL |
| | | CQAHGLTPEQVVAIASNIGGKQALETVQALLPVLCQAHGLTPQQVVAIASNN |
| | | GGKQALETVQRLLPVLCQAHGLTPEQVVAIASNIGGKQALETVQALLPVLCQ |
| | | AHGLTPEQVVAIASNIGGKQALETVQALLPVLCQAHGLTPQQVVAIASNNGG |
| | | KQALETVQRLLPVLCQAHGLTPEQVVAIASNIGGKQALETVQALLPVLCQAH |
| | | GLTPQQVVAIASNGGGKQALETVQRLLPVLCQAHGLTPQQVVAIASNGGGR |
| | | PALESIVAQLSRPDPALAALTNDHLVALACLGGRPALDAVKKGLGDPISRSQL |
| | | VKSELEEKKSELRHKLKYVPHEYIELIEIARNSTQDRILEMKVMEFFMKVYGY |
| | | RGKHLGGSRKPDGAIYTVGSPIDYGVIVDTKAYSGGYNLPIGQADEMQRYVE |
| | | ENQTRNKHINPNEWWKVYPSSVTEFKFLFVSGHFKGNYKAQLTRLNHITNC |
| | | NGAVLSVEELLIGGEMIKAGTLTLEEVRRKFNNGEINFAAD- |
| 38 | target | TCTAAGAAGTTCCTGCTCTGGAGTTGACTAAAGAATGTGGTTAGAGACA |
| | sequence | |
| | TALEN i7 | |
| 39 | target | TAGAAATTTGAGATTTTAGGAAGGGACTAGTAATAAAAGGTAAAATAAA |
| | sequence | |
| | TALEN i8b | |
| 40 | target | TGACTTCTGGTCATGGCCGTGGCGCGTGAGCCCATCTTCTCTTTCCTCA |
| | sequence | |
| | TALEN i9 | |
| 33 | STAT3 | TGATACAAGCTAGGCCCTTTTGCTAATCATGTTCATACTGTCCCTTTTTTT |
| | inserted | TCCACAGAGCATCGTGAGTGAGCTGGCTGGGTTGCTTTCAGCTATGGAA |
| | template | TATGTGCAAAAGACGTTGACAGACGAGGAACTGGCCGACTGGAAGCGGA |
| | sequence 1 | GGCAACAAATTGCTTGCATTGGAGGACCACCCAATATATGCCTGGATAGA |
| | (@ i7) | TTGGAGAACTGGATAACGTCATTAGCAGAAAGCCAACTGCAAACTAGACA |
| | | GCAAATAAAGAAGCTGGAAGAACTCCAGCAAAAAGTTTCCTATAAGGGTG |
| | | ACCCTATAGTGCAGCACAGGCCCATGTTGGAGGAACGCATTGTGGAGTT |
| | | GTTTAGAAATCTGATGAAAAGTGCCTTTGTGGTGGAGCGACAACCGTGCA |
| | | TGCCTATGCACCCTGACCGGCCCTTGGTGATAAAGACCGGAGTACAATT |
| | | CACTACTAAAGTGAGGCTGCTCGTAAAGTTCCCTGAACTCAATTACCAGT |
| | | TGAAGATAAAAGTGTGCATCGATAAGGACTCCGGCGATGTTGCCGCGCT |
| | | CCGGGGAAGTCGAAAGTTCAACATCTTGGGCACCAATACAAAGGTGATG |
| | | AACATGGAGGAATCAAACAACGGGTCCCTTTCCGCGGAGTTCAAACATTT |
| | | GACATTGCGCGAGCAGAGATGTGGGAACGGGGGAAGAGCAAATTGTGA |
| | | CGCTTCTCTGATCGTAACTGAGGAGCTGCATCTCATAACATTCGAGACTG |
| | | AGGTCTACCATCAGGGATTGAAGATCGACCTCGAAACTCATTCACTGCCT |
| | | GTAGTTGTTATTAGTAATATTTGCCAGATGCCGAATGCGTGGGCTAGCAT |
| | | ACTTTGGTATAACATGCTTACGAATAATCCAAAGAACGTTAATTTTTTCAC |
| | | TAAACCTCCAATCGGAACTTGGGACCAGGTCGCAGAAGTGCTGAGCTGG |
| | | CAATTTTCCTCCACCACCAAACGCGGTCTTAGCATCGAACAATTGACGAC |
| | | CCTCGCAGAAAAGCTTTTGGGTCCTGGCGTTAATTACTCAGGGTGTCAGA |
| | | TAACTTGGGCAAAGTTCTGCAAAGAAAACATGGCAGGTAAGGGCTTTTCA |
| | | TTTTGGGTCTGGCTGGATAACATTATAGATTTGGTGAAAAAGTACATTCTT |
| | | GCCCTGTGGAACGAAGGCTACATCATGGGTTTCATTAGCAAGGAGCGCG |
| | | AGAGAGCGATCCTGTCAACTAAACCCCCTGGAACGTTTCTCCTTAGATTT |
| | | TCCGAAAGCAGCAAGGAAGGTGGCGTGACTTTCACGTGGGTTGAAAAGG |
| | | ACATCAGCGGAAAGACGCAAATTCAGAGCGTTGAACCTTACACCAAGCA |
| | | GCAACTTAACAACATGAGCTTCGCCGAGATAATAATGGGTTACAAAATAA |
| | | TGGACGCTACTAATATCCTCGTCTCACCCCTCGTGTACTTGTATCCAGAC |
| | | ATTCCCAAAGAGGAGGCGTTCGGGAAGTACTGTCGCCCGGAGTCACAAG |
| | | AACATCCTGAAGCTGACCCAGGAAGTGCAGCTCCGTACCTTAAGACCAA |
| | | GTTTATCTGTGTGACACCGTAAGTGGCTTCCTTTCCCCGTTTTGCCTTCAT |
| | | TTCTAATATCCTCAGTTATCCCTGGGAATGGGACACTGGGTGAGAGTTAA |
| | | TCTGCCAAAGGTTGGAAGCCCCTGGGCTATGTTTAGTACTCAAAGTGACC |
| | | TTGTGTGTTTAAAAAGCTTGAGCTTTTATTTTTCTGTTGGAAGACCAGAGT |
| | | TTGATGGCTTGTGTGTGTGTGTTTTGTTCTTTTTTTTTTTTCCATTGTGTCT |
| | | TGTCAACCCCCCGTTTCCCCTCCTGCTGCCCCCCATTTCCTACAGAACGA |
| | | CCTGCAGCAATACCATTGACCTGCCGATGTCCCCCCGCACTTTAGATTCA |
| | | TTGATGCAGTTTGGAAATAATGGTGAAGGTGCTGAACCATCCGCTGGTG |
| | | GACAGTTTGAGAGCCTTACGTTTGATATGGAGTTGACAAGCGAGTGTGCT |
| | | ACATCACCTATGTGAGCAGATCCAGACATGATAAGATACATTGATGAGTT |
| | | TGGACAAACCACAACTAGAATGCAGTGAAAAAAATGCTTTATTTGTGAAAT |
| | | TTGTGATGCTATTGCTTTATTTGTAACCATTATAAGCTGCAATAAACAAGT |
| | | TAACAACAACAATTGCATTCATTTTATGTTTCAGGTTCAGGGGGAGGTGT |
| | | GGGAGGTTTTTTAAAGCAAGTAAAACCTCTACAAATGTGGTACGGCTTGA |
| | | TTATGATCA |
| 34 | STAT3 | TGATACAAGCTAGGCCCTTTTGCTAATCATGTTCATACTGTCCCTTTTTTT |
| | inserted | TCCTCAGAGCATCGTGAGTGAGCTGGCTGGGTTGCTTTCAGCTATGGAA |
| | template | TATGTGCAAAAGACGTTGACAGACGAGGAACTGGCCGACTGGAAGCGGA |
| | sequence | GGCAACAAATTGCTTGCATTGGAGGACCACCCAATATATGCCTGGATAGA |
| | 1bis (@ i7) | TTGGAGAACTGGATAACGTCATTAGCAGAAAGCCAACTGCAAACTAGACA |
| | pCLS37601 | GCAAATAAAGAAGCTGGAAGAACTCCAGCAAAAAGTTTCCTATAAGGGTG |
| | | ACCCTATAGTGCAGCACAGGCCCATGTTGGAGGAACGCATTGTGGAGTT |
| | | GTTTAGAAATCTGATGAAAAGTGCCTTTGTGGTGGAGCGACAACCGTGCA |
| | | TGCCTATGCACCCTGACCGGCCCTTGGTGATAAAGACCGGAGTACAATT |
| | | CACTACTAAAGTGAGGCTGCTCGTAAAGTTCCCTGAACTCAATTACCAGT |
| | | TGAAGATAAAAGTGTGCATCGATAAGGACTCCGGCGATGTTGCCGCGCT |
| | | CCGGGGAAGTCGAAAGTTCAACATCTTGGGCACCAATACAAAGGTGATG |
| | | AACATGGAGGAATCAAACAACGGGTCCCTTTCCGCGGAGTTCAAACATTT |
| | | GACATTGCGCGAGCAGAGATGTGGGAACGGGGGAAGAGCAAATTGTGA |
| | | CGCTTCTCTGATCGTAACTGAGGAGCTGCATCTCATAACATTCGAGACTG |
| | | AGGTCTACCATCAGGGATTGAAGATCGACCTCGAAACTCATTCACTGCCT |
| | | GTAGTTGTTATTAGTAATATTTGCCAGATGCCGAATGCGTGGGCTAGCAT |
| | | ACTTTGGTATAACATGCTTACGAATAATCCAAAGAACGTTAATTTTTTCAC |
| | | TAAACCTCCAATCGGAACTTGGGACCAGGTCGCAGAAGTGCTGAGCTGG |
| | | CAATTTTCCTCCACCACCAAACGCGGTCTTAGCATCGAACAATTGACGAC |
| | | CCTCGCAGAAAAGCTTTTGGGTCCTGGCGTTAATTACTCAGGGTGTCAGA |
| | | TAACTTGGGCAAAGTTCTGCAAAGAAAACATGGCAGGCAAGGGCTTTTCA |
| | | TTTTGGGTCTGGCTGGATAACATTATAGATTTGGTGAAAAAGTACATTCTT |
| | | GCCCTGTGGAACGAAGGCTACATCATGGGTTTCATTAGCAAGGAGCGCG |
| | | AGAGAGCGATCCTGTCAACTAAACCCCCTGGAACGTTTCTCCTTCGCTTT |
| | | TCCGAAAGCAGCAAGGAAGGTGGCGTGACTTTCACGTGGGTTGAAAAGG |
| | | ACATCAGCGGAAAGACGCAAATTCAGAGCGTTGAACCTTACACCAAGCA |
| | | GCAACTTAACAACATGAGCTTCGCCGAGATAATAATGGGTTACAAAATAA |
| | | TGGACGCTACTAATATCCTCGTCTCACCCCTCGTGTACTTGTATCCGGAC |
| | | ATTCCCAAAGAGGAGGCGTTCGGGAAGTACTGTCGCCCGGAGTCACAAG |
| | | AACATCCTGAAGCTGACCCAGGAAGTGCAGCTCCGTACCTTAAGACCAA |
| | | GTTTATCTGCGTGACACCGTAAGTGGCTTCCTTTCCCCGTTTTGCCTTCA |
| | | TTTCTAATATCCTCAGTTATCCCTGGGAATGGGACACTGGGTGAGAGTTA |
| | | ATCTGCCAAAGGTTGGAAGCCCCTGGGCTATGTTTAGTACTCAAAGTGAC |
| | | CTTGTGTGTTTAAAAAGCTTGAGCTTTTATTTTTCTGTTGGAGACCAGAGT |
| | | TTGATGGCTTGTGTGTGTGTGTTTTGTTCTTTTTTTTTTTTCCATTGTGTCT |
| | | TGTCAACCCCCCGTTTCCCCTCCTGCTGCCCCCCATTTCCTACAGAACGA |
| | | CCTGCAGCAATACCATTGACCTGCCGATGTCCCCCCGCACTTTAGATTCA |
| | | TTGATGCAGTTTGGAAATAATGGTGAAGGTGCTGAACCATCCGCTGGTG |
| | | GACAGTTTGAGAGCCTTACGTTTGATATGGAGTTGACAAGCGAGTGTGCT |
| | | ACATCACCTATGTGAGCAGATCCAGACATGATAAGATACATTGATGAGTT |
| | | TGGACAAACCACAACTAGAATGCAGTGAAAAAAATGCTTTATTTGTGAAAT |
| | | TTGTGATGCTATTGCTTTATTTGTAACCATTATAAGCTGCAATAAACAAGT |
| | | TAACAACAACAATTGCATTCATTTTATGTTTCAGGTTCAGGGGGAGGTGT |
| | | GGGAGGTTTTTTAAAGCAAGTAAAACCTCTACAAATGTGGTACGGCTTGA |
| | | TTATGATCA |
| 35 | STAT3 | TGATACAAGCTAGGCCCTTTTGCTAATCATGTTCATACTGTCCCTTTTTTT |
| | inserted | TCCTCAGAGCATCGTGAGTGAGCTGGCTGGGTTGCTTTCAGCTATGGAA |
| | template | TATGTGCAAAAGACGTTGACAGACGAGGAACTGGCCGACTGGAAGCGGA |
| | sequence | GGCAACAAATTGCTTGCATTGGAGGACCACCCAATATATGCCTGGATAGA |
| | 1tris (@ i7) | TTGGAGAACTGGATAACGTCATTAGCAGAAAGCCAACTGCAAACTAGACA |
| | pCLS37602 | GCAAATAAAGAAGCTGGAAGAACTCCAGCAAAAAGTTTCCTATAAGGGTG |
| | | ACCCTATAGTGCAGCACAGGCCCATGTTGGAGGAACGCATTGTGGAGTT |
| | | GTTTAGAAATCTGATGAAAAGTGCCTTTGTGGTGGAGCGACAACCGTGCA |
| | | TGCCTATGCACCCTGACCGGCCCTTGGTGATAAAGACCGGAGTACAATT |
| | | CACTACTAAAGTGAGGCTGCTCGTAAAGTTCCCTGAACTCAATTACCAGT |
| | | TGAAGATAAAAGTGTGCATCGATAAGGACTCCGGCGATGTTGCCGCGCT |
| | | CCGGGGAAGTCGAAAGTTCAACATCTTGGGCACCAATACAAAGGTGATG |
| | | AACATGGAGGAATCAAACAACGGGTCCCTTTCCGCGGAGTTCAAACATTT |
| | | GACATTGCGCGAGCAGAGATGTGGGAACGGGGGAAGAGCAAATTGTGA |
| | | CGCTTCTCTGATCGTAACTGAGGAGCTGCATCTCATAACATTCGAGACTG |
| | | AGGTCTACCATCAGGGATTGAAGATCGACCTCGAAACTCATTCACTGCCT |
| | | GTAGTTGTTATTAGTAATATTTGCCAGATGCCGAATGCGTGGGCTAGCAT |
| | | ACTTTGGTATAACATGCTTACGAATAATCCAAAGAACGTTAATTTTTTCAC |
| | | TAAACCTCCAATCGGAACTTGGGACCAGGTCGCAGAAGTGCTGAGCTGG |
| | | CAATTTTCCTCCACCACCAAACGCGGTCTTAGCATCGAACAATTGACGAC |
| | | CCTCGCAGAAAAGCTTTTGGGTCCTGGCGTTAATTACTCAGGGTGTCAGA |
| | | TAACTTGGGCAAAGTTCTGCAAAGAAAACATGGCAGGCAAGGGCTTTTCA |
| | | TTTTGGGTCTGGCTGGATAACATTATAGATTTGGTGAAAAAGTACATTCTT |
| | | GCCCTGTGGAACGAAGGCTACATCATGGGTTTCATTAGCAAGGAGCGCG |
| | | AGAGAGCGATCCTGTCAACTAAACCCCCTGGAACGTTTCTCCTTCGCTTT |
| | | TCCGAAAGCAGCAAGGAAGGTGGCGTGACTTTCACGTGGGTTGAAAAGG |
| | | ACATCAGCGGAAAGACGCAAATTCAGAGCGTTGAACCTTACACCAAGCA |
| | | GCAACTTAACAACATGAGCTTCGCCGAGATAATAATGGGTTACAAAATAA |
| | | TGGACGCTACTAATATCCTCGTCTCACCCCTCGTGTACTTGTATCCGGAC |
| | | ATTCCCAAAGAGGAGGCGTTCGGGAAGTACTGTCGCCCGGAGTCACAAG |
| | | AACATCCTGAAGCTGACCCAGGAAGTGCAGCTCCGTACCTTAAGACCAA |
| | | GTTTATCTGTGTGACACCGTAAGTGGCTTCCTTTCCCCGTTTTGCCTTCAT |
| | | TTCTAATATCCTCAGTTATCCCTGGGAATGGGACACTGGGTGAGAGTTAA |
| | | TCTGCCAAAGGTTGGAAGCCCCTGGGCTATGTTTAGTACTCAAAGTGACC |
| | | TTGTGTGTTTAAAAAGCTTGAGCTTTTATTTTTCTGTTGGAGACCAGAGTT |
| | | TGATGGCTTGTGTGTGTGTGTTTTGTTCTTTTTTTTTTTTCCATTGTGTCTT |
| | | GTCAACCCCCCGTTTCCCCTCCTGCTGCCCCCCATTTCCTACAGAACGA |
| | | CCTGCAGCAATACCATTGACCTGCCGATGTCCCCCCGCACTTTAGATTCA |
| | | TTGATGCAGTTTGGAAATAATGGTGAAGGTGCTGAACCATCCGCTGGTG |
| | | GACAGTTTGAGAGCCTTACGTTTGATATGGAGTTGACAAGCGAGTGTGCT |
| | | ACATCACCTATGTGAGCAGATCCAGACATGATAAGATACATTGATGAGTT |
| | | TGGACAAACCACAACTAGAATGCAGTGAAAAAAATGCTTTATTTGTGAAAT |
| | | TTGTGATGCTATTGCTTTATTTGTAACCATTATAAGCTGCAATAAACAAGT |
| | | TAACAACAACAATTGCATTCATTTTATGTTTCAGGTTCAGGGGGAGGTGT |
| | | GGGAGGTTTTTTAAAGCAAGTAAAACCTCTACAAATGTGGTACGGCTTGA |
| | | TTATGATCA |
| 36 | STAT3 | TGATACAAGCTAGGCCCTTTTGCTAATCATGTTCATACTGTCCCTTTTTTT |
| | inserted | TCCTCAGAGCATCGTGAGTGAGCTGGCGGGGCTTTTGTCAGCGATGGAG |
| | template | TACGTGCAGAAAACTCTCACGGACGAGGAGCTGGCTGACTGGAAGAGG |
| | sequence | CGGCAACAGATTGCCTGCATTGGAGGCCCGCCCAACATCTGCCTAGATC |
| | 1quater | GGCTAGAAAACTGGATAACGTCATTAGCAGAATCTCAACTTCAGACCCGT |
| | (@ i7) | CAACAAATTAAGAAACTGGAGGAGTTGCAGCAAAAAGTTTCCTACAAAGG |
| | pCLS37603 | GGACCCCATTGTACAGCACCGGCCGATGCTGGAGGAGAGAATCGTGGA |
| | | GCTGTTTAGAAACTTAATGAAAAGCGCCTTTGTGGTGGAGCGGCAGCCC |
| | | TGCATGCCCATGCATCCTGACCGGCCCCTCGTCATCAAGACCGGCGTCC |
| | | AGTTCACTACTAAAGTCAGATTGCTGGTCAAATTCCCTGAGTTGAATTATC |
| | | AGCTTAAAATTAAAGTGTGCATTGACAAAGACTCTGGGGACGTTGCAGCT |
| | | CTCAGAGGATCCCGGAAGTTCAACATCTTGGGCACCAACACAAAAGTGA |
| | | TGAACATGGAAGAATCCAACAACGGCAGCCTCTCTGCCGAATTCAAACAC |
| | | TTGACCCTGAGGGAGCAGAGATGTGGGAATGGGGGCCGAGCCAATTGT |
| | | GATGCTTCCCTGATTGTGACTGAGGAGCTGCACCTGATCACCTTTGAGAC |
| | | CGAGGTGTATCACCAAGGCCTCAAGATTGACCTAGAGACCCACTCCTTG |
| | | CCAGTTGTGGTGATCTCCAACATCTGTCAGATGCCAAATGCCTGGGCGT |
| | | CCATCCTGTGGTACAACATGCTGACCAACAATCCCAAGAATGTAAACTTT |
| | | TTTACCAAGCCCCCAATTGGAACCTGGGATCAAGTGGCCGAGGTCCTGA |
| | | GCTGGCAGTTCTCCTCCACCACCAAGCGAGGACTGAGCATCGAACAATT |
| | | GACGACCCTGGCAGAGAAACTCTTGGGACCTGGTGTGAATTATTCAGGG |
| | | TGTCAGATCACATGGGCTAAATTTTGCAAAGAAAACATGGCTGGCAAGGG |
| | | CTTCTCCTTCTGGGTCTGGCTGGACAATATCATTGACCTTGTGAAAAAGT |
| | | ACATCCTGGCCCTTTGGAACGAAGGATACATCATGGGCTTTATCAGTAAG |
| | | GAGCGGGAGAGAGCGATCCTGTCAACTAAACCTCCAGGCACCTTCCTGC |
| | | TAAGATTCAGTGAAAGCAGCAAAGAAGGAGGCGTCACTTTCACTTGGGT |
| | | GGAGAAGGACATCAGCGGAAAGACGCAAATTCAGAGCGTGGAACCATAC |
| | | ACAAAGCAGCAGCTGAACAACATGTCATTTGCTGAAATCATCATGGGCTA |
| | | TAAGATCATGGATGCTACCAATATCCTGGTGTCTCCACTGGTCTATCTCT |
| | | ATCCGGACATTCCCAAAGAGGAGGCGTTCGGAAAGTACTGTCGCCCGGA |
| | | GTCACAGGAGCATCCTGAAGCTGACCCAGGCAGCGCTGCCCCATACCTG |
| | | AAGACCAAGTTTATCTGTGTGACACCGTAAGTGGCTTCCTTTCCCCGTTT |
| | | TGCCTTCATTTCTAATATCCTCAGTTATCCCTGGGAATGGGACACTGGGT |
| | | GAGAGTTAATCTGCCAAAGGTTGGAAGCCCCTGGGCTATGTTTAGTACTC |
| | | AAAGTGACCTTGTGTGTTTAAAAAGCTTGAGCTTTTATTTTTCTGTTGGAG |
| | | ACCAGAGTTTGATGGCTTGTGTGTGTGTGTTTTGTTCTTTTTTTTTTTTCCA |
| | | TTGTGTCTTGTCAACCCCCCGTTTCCCCTCCTGCTGCCCCCCATTTCCTA |
| | | CAGAACGACCTGCAGCAATACCATTGACCTGCCGATGTCCCCCCGCACT |
| | | TTAGATTCATTGATGCAGTTTGGAAATAATGGTGAAGGTGCTGAACCCTC |
| | | AGCAGGAGGACAGTTTGAGAGCCTTACGTTTGATATGGAGTTGACCTCG |
| | | GAGTGCGCTACCTCCCCCATGTGAGCAGATCCAGACATGATAAGATACA |
| | | TTGATGAGTTTGGACAAACCACAACTAGAATGCAGTGAAAAAAATGCTTT |
| | | ATTTGTGAAATTTGTGATGCTATTGCTTTATTTGTAACCATTATAAGCTGCA |
| | | ATAAACAAGTTAACAACAACAATTGCATTCATTTTATGTTTCAGGTTCAGG |
| | | GGGAGGTGTGGGAGGTTTTTTAAAGCAAGTAAAACCTCTACAAATGTGGT |
| | | ACGGCTTGATTATGATCA |
| 37 | STAT3 | TGATACAAGCTAGGCCCTTTTGCTAATCATGTTCATACTGTCCCTTTTTTT |
| | inserted | TCCTCAGAGCATCGTGAGTGAGCTGGCTGGGTTGCTTTCAGCTATGGAA |
| | template | TATGTGCAAAAGACGTTGACAGACGAGGAACTGGCCGACTGGAAGCGGA |
| | sequence | GGCAACAAATTGCTTGCATTGGAGGACCACCCAATATATGCCTGGATAGA |
| | 1penta (@ i7) | TTGGAGAACTGGATAACGTCATTAGCAGAAAGCCAACTGCAAACTAGACA |
| | pCLS37604 | GCAAATAAAGAAGCTGGAAGAACTCCAGCAAAAAGTTTCCTATAAGGGTG |
| | | ACCCTATAGTGCAGCACAGGCCCATGTTGGAGGAACGCATTGTGGAGTT |
| | | GTTTAGAAATCTGATGAAAAGTGCCTTTGTGGTGGAGCGACAACCGTGCA |
| | | TGCCTATGCACCCTGACCGGCCCTTGGTGATAAAGACCGGAGTACAATT |
| | | CACTACTAAAGTGAGGCTGCTCGTAAAGTTCCCTGAACTCAATTACCAGT |
| | | TGAAGATAAAAGTGTGCATCGATAAAGACTCTGGGGACGTTGCAGCTCTC |
| | | AGAGGATCCCGGAAGTTCAACATCTTGGGCACCAACACAAAAGTGATGA |
| | | ACATGGAAGAATCCAACAACGGCAGCCTCTCTGCCGAATTCAAACACTTG |
| | | ACCCTGAGGGAGCAGAGATGTGGGAATGGGGGCCGAGCCAATTGTGAT |
| | | GCTTCCCTGATTGTGACTGAGGAGCTGCACCTGATCACCTTTGAGACCG |
| | | AGGTGTATCACCAAGGCCTCAAGATTGACCTAGAGACCCACTCCTTGCC |
| | | AGTTGTGGTGATCTCCAACATCTGTCAGATGCCAAATGCCTGGGCGTCC |
| | | ATCCTGTGGTACAACATGCTGACCAACAATCCCAAGAATGTAAACTTTTTT |
| | | ACCAAGCCCCCAATTGGAACCTGGGATCAAGTGGCCGAGGTCCTGAGCT |
| | | GGCAGTTCTCCTCCACCACCAAGCGAGGACTGAGCATCGAACAATTGAC |
| | | GACCCTGGCAGAGAAACTCTTGGGACCTGGTGTGAATTATTCAGGGTGT |
| | | CAGATCACATGGGCTAAATTTTGCAAAGAAAACATGGCTGGCAAGGGCTT |
| | | CTCCTTCTGGGTCTGGCTGGACAATATCATTGACCTTGTGAAAAAGTACA |
| | | TCCTGGCCCTTTGGAACGAAGGATACATCATGGGCTTTATCAGTAAGGAG |
| | | CGGGAGAGAGCGATCCTGTCAACTAAACCTCCAGGCACCTTCCTGCTAA |
| | | GATTCAGTGAAAGCAGCAAAGAAGGAGGCGTCACTTTCACTTGGGTGGA |
| | | GAAGGACATCAGCGGAAAGACGCAAATTCAGAGCGTGGAACCATACACA |
| | | AAGCAGCAGCTGAACAACATGTCATTTGCTGAAATCATCATGGGCTATAA |
| | | GATCATGGATGCTACCAATATCCTGGTGTCTCCACTGGTCTATCTCTATC |
| | | CGGACATTCCCAAAGAGGAGGCGTTCGGAAAGTACTGTCGCCCGGAGTC |
| | | ACAGGAGCATCCTGAAGCTGACCCAGGCAGCGCTGCCCCATACCTGAAG |
| | | ACCAAGTTTATCTGTGTGACACCGTAAGTGGCTTCCTTTCCCCGTTTTGC |
| | | CTTCATTTCTAATATCCTCAGTTATCCCTGGGAATGGGACACTGGGTGAG |
| | | AGTTAATCTGCCAAAGGTTGGAAGCCCCTGGGCTATGTTTAGTACTCAAA |
| | | GTGACCTTGTGTGTTTAAAAAGCTTGAGCTTTTATTTTTCTGTTGGAGACC |
| | | AGAGTTTGATGGCTTGTGTGTGTGTGTTTTGTTCTTTTTTTTTTTTCCATTG |
| | | TGTCTTGTCAACCCCCCGTTTCCCCTCCTGCTGCCCCCCATTTCCTACAG |
| | | AACGACCTGCAGCAATACCATTGACCTGCCGATGTCCCCCCGCACTTTA |
| | | GATTCATTGATGCAGTTTGGAAATAATGGTGAAGGTGCTGAACCCTCAGC |
| | | AGGAGGACAGTTTGAGAGCCTTACGTTTGATATGGAGTTGACCTCGGAG |
| | | TGCGCTACCTCCCCCATGTGAGCAGATCCAGACATGATAAGATACATTGA |
| | | TGAGTTTGGACAAACCACAACTAGAATGCAGTGAAAAAAATGCTTTATTTG |
| | | TGAAATTTGTGATGCTATTGCTTTATTTGTAACCATTATAAGCTGCAATAAA |
| | | CAAGTTAACAACAACAATTGCATTCATTTTATGTTTCAGGTTCAGGGGGA |
| | | GGTGTGGGAGGTTTTTTAAAGCAAGTAAAACCTCTACAAATGTGGTACGG |
| | | CTTGATTATGATCA |
| 41 | STAT3 with | GTCAAGGAGTATTCCCTCAGGTCAAGGAGTTTTTTCTTCCTTCGCAGACA |
| | homologies | TGCAAGATCTGAATGGAAACAACCAGTCAGTGACCAGGCAGAAGATGCA |
| | template | GCAGCTGGAACAGATGCTCACTGCGCTGGACCAGATGCGGAGAGTAAG |
| | sequence 1 | GGCATAGGTCGGACCACTTCCCCCATGTGTCTCGCTCACTTGCGGGATT |
| | (@ i7) | TCAGCGTCTTGTGGCAGAACTTGCTTGGTTTCTAAGAAGTTCCTGCTCTG |
| | pCLS35053 | GAGTTGATACAAGCTAGGCCCTTTTGCTAATCATGTTCATACTGTCCCTTT |
| | | TTTTTCCACAGAGCATCGTGAGTGAGCTGGCTGGGTTGCTTTCAGCTATG |
| | | GAATATGTGCAAAAGACGTTGACAGACGAGGAACTGGCCGACTGGAAGC |
| | | GGAGGCAACAAATTGCTTGCATTGGAGGACCACCCAATATATGCCTGGA |
| | | TAGATTGGAGAACTGGATAACGTCATTAGCAGAAAGCCAACTGCAAACTA |
| | | GACAGCAAATAAAGAAGCTGGAAGAACTCCAGCAAAAAGTTTCCTATAAG |
| | | GGTGACCCTATAGTGCAGCACAGGCCCATGTTGGAGGAACGCATTGTGG |
| | | AGTTGTTTAGAAATCTGATGAAAAGTGCCTTTGTGGTGGAGCGACAACCG |
| | | TGCATGCCTATGCACCCTGACCGGCCCTTGGTGATAAAGACCGGAGTAC |
| | | AATTCACTACTAAAGTGAGGCTGCTCGTAAAGTTCCCTGAACTCAATTAC |
| | | CAGTTGAAGATAAAAGTGTGCATCGATAAGGACTCCGGCGATGTTGCCG |
| | | CGCTCCGGGGAAGTCGAAAGTTCAACATCTTGGGCACCAATACAAAGGT |
| | | GATGAACATGGAGGAATCAAACAACGGGTCCCTTTCCGCGGAGTTCAAA |
| | | CATTTGACATTGCGCGAGCAGAGATGTGGGAACGGGGGAAGAGCAAATT |
| | | GTGACGCTTCTCTGATCGTAACTGAGGAGCTGCATCTCATAACATTCGAG |
| | | ACTGAGGTCTACCATCAGGGATTGAAGATCGACCTCGAAACTCATTCACT |
| | | GCCTGTAGTTGTTATTAGTAATATTTGCCAGATGCCGAATGCGTGGGCTA |
| | | GCATACTTTGGTATAACATGCTTACGAATAATCCAAAGAACGTTAATTTTT |
| | | TCACTAAACCTCCAATCGGAACTTGGGACCAGGTCGCAGAAGTGCTGAG |
| | | CTGGCAATTTTCCTCCACCACCAAACGCGGTCTTAGCATCGAACAATTGA |
| | | CGACCCTCGCAGAAAAGCTTTTGGGTCCTGGCGTTAATTACTCAGGGTG |
| | | TCAGATAACTTGGGCAAAGTTCTGCAAAGAAAACATGGCAGGTAAGGGC |
| | | TTTTCATTTTGGGTCTGGCTGGATAACATTATAGATTTGGTGAAAAAGTAC |
| | | ATTCTTGCCCTGTGGAACGAAGGCTACATCATGGGTTTCATTAGCAAGGA |
| | | GCGCGAGAGAGCGATCCTGTCAACTAAACCCCCTGGAACGTTTCTCCTT |
| | | AGATTTTCCGAAAGCAGCAAGGAAGGTGGCGTGACTTTCACGTGGGTTG |
| | | AAAAGGACATCAGCGGAAAGACGCAAATTCAGAGCGTTGAACCTTACAC |
| | | CAAGCAGCAACTTAACAACATGAGCTTCGCCGAGATAATAATGGGTTACA |
| | | AAATAATGGACGCTACTAATATCCTCGTCTCACCCCTCGTGTACTTGTATC |
| | | CAGACATTCCCAAAGAGGAGGCGTTCGGGAAGTACTGTCGCCCGGAGTC |
| | | ACAAGAACATCCTGAAGCTGACCCAGGAAGTGCAGCTCCGTACCTTAAG |
| | | ACCAAGTTTATCTGTGTGACACCGTAAGTGGCTTCCTTTCCCCGTTTTGC |
| | | CTTCATTTCTAATATCCTCAGTTATCCCTGGGAATGGGACACTGGGTGAG |
| | | AGTTAATCTGCCAAAGGTTGGAAGCCCCTGGGCTATGTTTAGTACTCAAA |
| | | GTGACCTTGTGTGTTTAAAAAGCTTGAGCTTTTATTTTTCTGTTGGAAGAC |
| | | CAGAGTTTGATGGCTTGTGTGTGTGTGTTTTGTTCTTTTTTTTTTTTCCATT |
| | | GTGTCTTGTCAACCCCCCGTTTCCCCTCCTGCTGCCCCCCATTTCCTACA |
| | | GAACGACCTGCAGCAATACCATTGACCTGCCGATGTCCCCCCGCACTTT |
| | | AGATTCATTGATGCAGTTTGGAAATAATGGTGAAGGTGCTGAACCATCCG |
| | | CTGGTGGACAGTTTGAGAGCCTTACGTTTGATATGGAGTTGACAAGCGA |
| | | GTGTGCTACATCACCTATGTGAGCAGATCCAGACATGATAAGATACATTG |
| | | ATGAGTTTGGACAAACCACAACTAGAATGCAGTGAAAAAAATGCTTTATTT |
| | | GTGAAATTTGTGATGCTATTGCTTTATTTGTAACCATTATAAGCTGCAATA |
| | | AACAAGTTAACAACAACAATTGCATTCATTTTATGTTTCAGGTTCAGGGGG |
| | | AGGTGTGGGAGGTTTTTTAAAGCAAGTAAAACCTCTACAAATGTGGTACG |
| | | GCTTGATTATGATCATGACTAAAGAATGTGGTTAGAGACAGTCTGAGGAA |
| | | ATGTTTTCTGACTTTGTTTTGGTTTCCAACCAGAGCATCGTGAGTGAGCT |
| | | GGCGGGGCTTTTGTCAGCGATGGAGTACGTGCAGAAAACTCTCACGGAC |
| | | GAGGAGCTGGCTGACTGGAAGAGGCGGCAACAGATTGCCTGCATTGGA |
| | | GGCCCGCCCAACATCTGCCTAGATCGGCTAGAAAACTGGTAAAGGATGA |
| | | AAGAAGCTTTTCCTTTCTTTCTCGAAAGCTAGATTGAATTCTGATCTTAAC |
| | | TGCAGGCCCACAGAATTGGTACTATATCTCCAACGTGGGGACTTTTCCAT |
| | | ATTCAAATTTAGCCCAAGAATTAAAGTTTTTACTTTATTTCGGCCAGGCGC |
| | | TGTGGCTCACACCTGTAATCCCAGCACTTTGGGAGA |
| 42 | STAT3 with | GTCAAGGAGTATTCCCTCAGGTCAAGGAGTTTTTTCTTCCTTCGCAGACA |
| | homologies | TGCAAGATCTGAATGGAAACAACCAGTCAGTGACCAGGCAGAAGATGCA |
| | template | GCAGCTGGAACAGATGCTCACTGCGCTGGACCAGATGCGGAGAGTAAG |
| | sequence | GGCATAGGTCGGACCACTTCCCCCATGTGTCTCGCTCACTTGCGGGATT |
| | 1bis (@ i7) | TCAGCGTCTTGTGGCAGAACTTGCTTGGTTTCTAAGAAGTTCCTGCTCTG |
| | pCLS37601 | GAGTTGATACAAGCTAGGCCCTTTTGCTAATCATGTTCATACTGTCCCTTT |
| | | TTTTTCCTCAGAGCATCGTGAGTGAGCTGGCTGGGTTGCTTTCAGCTATG |
| | | GAATATGTGCAAAAGACGTTGACAGACGAGGAACTGGCCGACTGGAAGC |
| | | GGAGGCAACAAATTGCTTGCATTGGAGGACCACCCAATATATGCCTGGA |
| | | TAGATTGGAGAACTGGATAACGTCATTAGCAGAAAGCCAACTGCAAACTA |
| | | GACAGCAAATAAAGAAGCTGGAAGAACTCCAGCAAAAAGTTTCCTATAAG |
| | | GGTGACCCTATAGTGCAGCACAGGCCCATGTTGGAGGAACGCATTGTGG |
| | | AGTTGTTTAGAAATCTGATGAAAAGTGCCTTTGTGGTGGAGCGACAACCG |
| | | TGCATGCCTATGCACCCTGACCGGCCCTTGGTGATAAAGACCGGAGTAC |
| | | AATTCACTACTAAAGTGAGGCTGCTCGTAAAGTTCCCTGAACTCAATTAC |
| | | CAGTTGAAGATAAAAGTGTGCATCGATAAGGACTCCGGCGATGTTGCCG |
| | | CGCTCCGGGGAAGTCGAAAGTTCAACATCTTGGGCACCAATACAAAGGT |
| | | GATGAACATGGAGGAATCAAACAACGGGTCCCTTTCCGCGGAGTTCAAA |
| | | CATTTGACATTGCGCGAGCAGAGATGTGGGAACGGGGGAAGAGCAAATT |
| | | GTGACGCTTCTCTGATCGTAACTGAGGAGCTGCATCTCATAACATTCGAG |
| | | ACTGAGGTCTACCATCAGGGATTGAAGATCGACCTCGAAACTCATTCACT |
| | | GCCTGTAGTTGTTATTAGTAATATTTGCCAGATGCCGAATGCGTGGGCTA |
| | | GCATACTTTGGTATAACATGCTTACGAATAATCCAAAGAACGTTAATTTTT |
| | | TCACTAAACCTCCAATCGGAACTTGGGACCAGGTCGCAGAAGTGCTGAG |
| | | CTGGCAATTTTCCTCCACCACCAAACGCGGTCTTAGCATCGAACAATTGA |
| | | CGACCCTCGCAGAAAAGCTTTTGGGTCCTGGCGTTAATTACTCAGGGTG |
| | | TCAGATAACTTGGGCAAAGTTCTGCAAAGAAAACATGGCAGGCAAGGGC |
| | | TTTTCATTTTGGGTCTGGCTGGATAACATTATAGATTTGGTGAAAAAGTAC |
| | | ATTCTTGCCCTGTGGAACGAAGGCTACATCATGGGTTTCATTAGCAAGGA |
| | | GCGCGAGAGAGCGATCCTGTCAACTAAACCCCCTGGAACGTTTCTCCTT |
| | | CGCTTTTCCGAAAGCAGCAAGGAAGGTGGCGTGACTTTCACGTGGGTTG |
| | | AAAAGGACATCAGCGGAAAGACGCAAATTCAGAGCGTTGAACCTTACAC |
| | | CAAGCAGCAACTTAACAACATGAGCTTCGCCGAGATAATAATGGGTTACA |
| | | AAATAATGGACGCTACTAATATCCTCGTCTCACCCCTCGTGTACTTGTATC |
| | | CGGACATTCCCAAAGAGGAGGCGTTCGGGAAGTACTGTCGCCCGGAGT |
| | | CACAAGAACATCCTGAAGCTGACCCAGGAAGTGCAGCTCCGTACCTTAA |
| | | GACCAAGTTTATCTGCGTGACACCGTAAGTGGCTTCCTTTCCCCGTTTTG |
| | | CCTTCATTTCTAATATCCTCAGTTATCCCTGGGAATGGGACACTGGGTGA |
| | | GAGTTAATCTGCCAAAGGTTGGAAGCCCCTGGGCTATGTTTAGTACTCAA |
| | | AGTGACCTTGTGTGTTTAAAAAGCTTGAGCTTTTATTTTTCTGTTGGAGAC |
| | | CAGAGTTTGATGGCTTGTGTGTGTGTGTTTTGTTCTTTTTTTTTTTTCCATT |
| | | GTGTCTTGTCAACCCCCCGTTTCCCCTCCTGCTGCCCCCCATTTCCTACA |
| | | GAACGACCTGCAGCAATACCATTGACCTGCCGATGTCCCCCCGCACTTT |
| | | AGATTCATTGATGCAGTTTGGAAATAATGGTGAAGGTGCTGAACCATCCG |
| | | CTGGTGGACAGTTTGAGAGCCTTACGTTTGATATGGAGTTGACAAGCGA |
| | | GTGTGCTACATCACCTATGTGAGCAGATCCAGACATGATAAGATACATTG |
| | | ATGAGTTTGGACAAACCACAACTAGAATGCAGTGAAAAAAATGCTTTATTT |
| | | GTGAAATTTGTGATGCTATTGCTTTATTTGTAACCATTATAAGCTGCAATA |
| | | AACAAGTTAACAACAACAATTGCATTCATTTTATGTTTCAGGTTCAGGGGG |
| | | AGGTGTGGGAGGTTTTTTAAAGCAAGTAAAACCTCTACAAATGTGGTACG |
| | | GCTTGATTATGATCATGACTAAAGAATGTGGTTAGAGACAGTCTGAGGAA |
| | | ATGTTTTCTGACTTTGTTTTGGTTTCCAACCAGAGCATCGTGAGTGAGCT |
| | | GGCGGGGCTTTTGTCAGCGATGGAGTACGTGCAGAAAACTCTCACGGAC |
| | | GAGGAGCTGGCTGACTGGAAGAGGCGGCAACAGATTGCCTGCATTGGA |
| | | GGCCCGCCCAACATCTGCCTAGATCGGCTAGAAAACTGGTAAAGGATGA |
| | | AAGAAGCTTTTCCTTTCTTTCTCGAAAGCTAGATTGAATTCTGATCTTAAC |
| | | TGCAGGCCCACAGAATTGGTACTATATCTCCAACGTGGGGACTTTTCCAT |
| | | ATTCAAATTTAGCCCAAGAATTAAAGTTTTTACTTTATTTCGGCCAGGCGC |
| | | TGTGGCTCACACCTGTAATCCCAGCACTTTGGGAGA |
| 43 | STAT3 with | GTCAAGGAGTATTCCCTCAGGTCAAGGAGTTTTTTCTTCCTTCGCAGACA |
| | homologies | TGCAAGATCTGAATGGAAACAACCAGTCAGTGACCAGGCAGAAGATGCA |
| | template | GCAGCTGGAACAGATGCTCACTGCGCTGGACCAGATGCGGAGAGTAAG |
| | sequence | GGCATAGGTCGGACCACTTCCCCCATGTGTCTCGCTCACTTGCGGGATT |
| | 1tris (@ i7) | TCAGCGTCTTGTGGCAGAACTTGCTTGGTTTCTAAGAAGTTCCTGCTCTG |
| | pCLS37602 | GAGTTGATACAAGCTAGGCCCTTTTGCTAATCATGTTCATACTGTCCCTTT |
| | | TTTTTCCTCAGAGCATCGTGAGTGAGCTGGCTGGGTTGCTTTCAGCTATG |
| | | GAATATGTGCAAAAGACGTTGACAGACGAGGAACTGGCCGACTGGAAGC |
| | | GGAGGCAACAAATTGCTTGCATTGGAGGACCACCCAATATATGCCTGGA |
| | | TAGATTGGAGAACTGGATAACGTCATTAGCAGAAAGCCAACTGCAAACTA |
| | | GACAGCAAATAAAGAAGCTGGAAGAACTCCAGCAAAAAGTTTCCTATAAG |
| | | GGTGACCCTATAGTGCAGCACAGGCCCATGTTGGAGGAACGCATTGTGG |
| | | AGTTGTTTAGAAATCTGATGAAAAGTGCCTTTGTGGTGGAGCGACAACCG |
| | | TGCATGCCTATGCACCCTGACCGGCCCTTGGTGATAAAGACCGGAGTAC |
| | | AATTCACTACTAAAGTGAGGCTGCTCGTAAAGTTCCCTGAACTCAATTAC |
| | | CAGTTGAAGATAAAAGTGTGCATCGATAAGGACTCCGGCGATGTTGCCG |
| | | CGCTCCGGGGAAGTCGAAAGTTCAACATCTTGGGCACCAATACAAAGGT |
| | | GATGAACATGGAGGAATCAAACAACGGGTCCCTTTCCGCGGAGTTCAAA |
| | | CATTTGACATTGCGCGAGCAGAGATGTGGGAACGGGGGAAGAGCAAATT |
| | | GTGACGCTTCTCTGATCGTAACTGAGGAGCTGCATCTCATAACATTCGAG |
| | | ACTGAGGTCTACCATCAGGGATTGAAGATCGACCTCGAAACTCATTCACT |
| | | GCCTGTAGTTGTTATTAGTAATATTTGCCAGATGCCGAATGCGTGGGCTA |
| | | GCATACTTTGGTATAACATGCTTACGAATAATCCAAAGAACGTTAATTTTT |
| | | TCACTAAACCTCCAATCGGAACTTGGGACCAGGTCGCAGAAGTGCTGAG |
| | | CTGGCAATTTTCCTCCACCACCAAACGCGGTCTTAGCATCGAACAATTGA |
| | | CGACCCTCGCAGAAAAGCTTTTGGGTCCTGGCGTTAATTACTCAGGGTG |
| | | TCAGATAACTTGGGCAAAGTTCTGCAAAGAAAACATGGCAGGCAAGGGC |
| | | TTTTCATTTTGGGTCTGGCTGGATAACATTATAGATTTGGTGAAAAAGTAC |
| | | ATTCTTGCCCTGTGGAACGAAGGCTACATCATGGGTTTCATTAGCAAGGA |
| | | GCGCGAGAGAGCGATCCTGTCAACTAAACCCCCTGGAACGTTTCTCCTT |
| | | CGCTTTTCCGAAAGCAGCAAGGAAGGTGGCGTGACTTTCACGTGGGTTG |
| | | AAAAGGACATCAGCGGAAAGACGCAAATTCAGAGCGTTGAACCTTACAC |
| | | CAAGCAGCAACTTAACAACATGAGCTTCGCCGAGATAATAATGGGTTACA |
| | | AAATAATGGACGCTACTAATATCCTCGTCTCACCCCTCGTGTACTTGTATC |
| | | CGGACATTCCCAAAGAGGAGGCGTTCGGGAAGTACTGTCGCCCGGAGT |
| | | CACAAGAACATCCTGAAGCTGACCCAGGAAGTGCAGCTCCGTACCTTAA |
| | | GACCAAGTTTATCTGTGTGACACCGTAAGTGGCTTCCTTTCCCCGTTTTG |
| | | CCTTCATTTCTAATATCCTCAGTTATCCCTGGGAATGGGACACTGGGTGA |
| | | GAGTTAATCTGCCAAAGGTTGGAAGCCCCTGGGCTATGTTTAGTACTCAA |
| | | AGTGACCTTGTGTGTTTAAAAAGCTTGAGCTTTTATTTTTCTGTTGGAGAC |
| | | CAGAGTTTGATGGCTTGTGTGTGTGTGTTTTGTTCTTTTTTTTTTTTCCATT |
| | | GTGTCTTGTCAACCCCCCGTTTCCCCTCCTGCTGCCCCCCATTTCCTACA |
| | | GAACGACCTGCAGCAATACCATTGACCTGCCGATGTCCCCCCGCACTTT |
| | | AGATTCATTGATGCAGTTTGGAAATAATGGTGAAGGTGCTGAACCATCCG |
| | | CTGGTGGACAGTTTGAGAGCCTTACGTTTGATATGGAGTTGACAAGCGA |
| | | GTGTGCTACATCACCTATGTGAGCAGATCCAGACATGATAAGATACATTG |
| | | ATGAGTTTGGACAAACCACAACTAGAATGCAGTGAAAAAAATGCTTTATTT |
| | | GTGAAATTTGTGATGCTATTGCTTTATTTGTAACCATTATAAGCTGCAATA |
| | | AACAAGTTAACAACAACAATTGCATTCATTTTATGTTTCAGGTTCAGGGGG |
| | | AGGTGTGGGAGGTTTTTTAAAGCAAGTAAAACCTCTACAAATGTGGTACG |
| | | GCTTGATTATGATCATGACTAAAGAATGTGGTTAGAGACAGTCTGAGGAA |
| | | ATGTTTTCTGACTTTGTTTTGGTTTCCAACCAGAGCATCGTGAGTGAGCT |
| | | GGCGGGGCTTTTGTCAGCGATGGAGTACGTGCAGAAAACTCTCACGGAC |
| | | GAGGAGCTGGCTGACTGGAAGAGGCGGCAACAGATTGCCTGCATTGGA |
| | | GGCCCGCCCAACATCTGCCTAGATCGGCTAGAAAACTGGTAAAGGATGA |
| | | AAGAAGCTTTTCCTTTCTTTCTCGAAAGCTAGATTGAATTCTGATCTTAAC |
| | | TGCAGGCCCACAGAATTGGTACTATATCTCCAACGTGGGGACTTTTCCAT |
| | | ATTCAAATTTAGCCCAAGAATTAAAGTTTTTACTTTATTTCGGCCAGGCGC |
| | | TGTGGCTCACACCTGTAATCCCAGCACTTTGGGAGA |
| 44 | STAT3 with | GTCAAGGAGTATTCCCTCAGGTCAAGGAGTTTTTTCTTCCTTCGCAGACA |
| | homologies | TGCAAGATCTGAATGGAAACAACCAGTCAGTGACCAGGCAGAAGATGCA |
| | template | GCAGCTGGAACAGATGCTCACTGCGCTGGACCAGATGCGGAGAGTAAG |
| | sequence | GGCATAGGTCGGACCACTTCCCCCATGTGTCTCGCTCACTTGCGGGATT |
| | 1 quater | TCAGCGTCTTGTGGCAGAACTTGCTTGGTTTCTAAGAAGTTCCTGCTCTG |
| | (@ i7) | GAGTTGATACAAGCTAGGCCCTTTTGCTAATCATGTTCATACTGTCCCTTT |
| | pCLS37603 | TTTTTCCTCAGAGCATCGTGAGTGAGCTGGCGGGGCTTTTGTCAGCGAT |
| | | GGAGTACGTGCAGAAAACTCTCACGGACGAGGAGCTGGCTGACTGGAA |
| | | GAGGCGGCAACAGATTGCCTGCATTGGAGGCCCGCCCAACATCTGCCTA |
| | | GATCGGCTAGAAAACTGGATAACGTCATTAGCAGAATCTCAACTTCAGAC |
| | | CCGTCAACAAATTAAGAAACTGGAGGAGTTGCAGCAAAAAGTTTCCTACA |
| | | AAGGGGACCCCATTGTACAGCACCGGCCGATGCTGGAGGAGAGAATCG |
| | | TGGAGCTGTTTAGAAACTTAATGAAAAGCGCCTTTGTGGTGGAGCGGCA |
| | | GCCCTGCATGCCCATGCATCCTGACCGGCCCCTCGTCATCAAGACCGGC |
| | | GTCCAGTTCACTACTAAAGTCAGATTGCTGGTCAAATTCCCTGAGTTGAA |
| | | TTATCAGCTTAAAATTAAAGTGTGCATTGACAAAGACTCTGGGGACGTTG |
| | | CAGCTCTCAGAGGATCCCGGAAGTTCAACATCTTGGGCACCAACACAAA |
| | | AGTGATGAACATGGAAGAATCCAACAACGGCAGCCTCTCTGCCGAATTC |
| | | AAACACTTGACCCTGAGGGAGCAGAGATGTGGGAATGGGGGCCGAGCC |
| | | AATTGTGATGCTTCCCTGATTGTGACTGAGGAGCTGCACCTGATCACCTT |
| | | TGAGACCGAGGTGTATCACCAAGGCCTCAAGATTGACCTAGAGACCCAC |
| | | TCCTTGCCAGTTGTGGTGATCTCCAACATCTGTCAGATGCCAAATGCCTG |
| | | GGCGTCCATCCTGTGGTACAACATGCTGACCAACAATCCCAAGAATGTAA |
| | | ACTTTTTTACCAAGCCCCCAATTGGAACCTGGGATCAAGTGGCCGAGGT |
| | | CCTGAGCTGGCAGTTCTCCTCCACCACCAAGCGAGGACTGAGCATCGAA |
| | | CAATTGACGACCCTGGCAGAGAAACTCTTGGGACCTGGTGTGAATTATTC |
| | | AGGGTGTCAGATCACATGGGCTAAATTTTGCAAAGAAAACATGGCTGGCA |
| | | AGGGCTTCTCCTTCTGGGTCTGGCTGGACAATATCATTGACCTTGTGAAA |
| | | AAGTACATCCTGGCCCTTTGGAACGAAGGATACATCATGGGCTTTATCAG |
| | | TAAGGAGCGGGAGAGAGCGATCCTGTCAACTAAACCTCCAGGCACCTTC |
| | | CTGCTAAGATTCAGTGAAAGCAGCAAAGAAGGAGGCGTCACTTTCACTTG |
| | | GGTGGAGAAGGACATCAGCGGAAAGACGCAAATTCAGAGCGTGGAACC |
| | | ATACACAAAGCAGCAGCTGAACAACATGTCATTTGCTGAAATCATCATGG |
| | | GCTATAAGATCATGGATGCTACCAATATCCTGGTGTCTCCACTGGTCTAT |
| | | CTCTATCCGGACATTCCCAAAGAGGAGGCGTTCGGAAAGTACTGTCGCC |
| | | CGGAGTCACAGGAGCATCCTGAAGCTGACCCAGGCAGCGCTGCCCCAT |
| | | ACCTGAAGACCAAGTTTATCTGTGTGACACCGTAAGTGGCTTCCTTTCCC |
| | | CGTTTTGCCTTCATTTCTAATATCCTCAGTTATCCCTGGGAATGGGACACT |
| | | GGGTGAGAGTTAATCTGCCAAAGGTTGGAAGCCCCTGGGCTATGTTTAG |
| | | TACTCAAAGTGACCTTGTGTGTTTAAAAAGCTTGAGCTTTTATTTTTCTGTT |
| | | GGAGACCAGAGTTTGATGGCTTGTGTGTGTGTGTTTTGTTCTTTTTTTTTT |
| | | TTCCATTGTGTCTTGTCAACCCCCCGTTTCCCCTCCTGCTGCCCCCCATT |
| | | TCCTACAGAACGACCTGCAGCAATACCATTGACCTGCCGATGTCCCCCC |
| | | GCACTTTAGATTCATTGATGCAGTTTGGAAATAATGGTGAAGGTGCTGAA |
| | | CCCTCAGCAGGAGGACAGTTTGAGAGCCTTACGTTTGATATGGAGTTGA |
| | | CCTCGGAGTGCGCTACCTCCCCCATGTGAGCAGATCCAGACATGATAAG |
| | | ATACATTGATGAGTTTGGACAAACCACAACTAGAATGCAGTGAAAAAAAT |
| | | GCTTTATTTGTGAAATTTGTGATGCTATTGCTTTATTTGTAACCATTATAAG |
| | | CTGCAATAAACAAGTTAACAACAACAATTGCATTCATTTTATGTTTCAGGT |
| | | TCAGGGGGAGGTGTGGGAGGTTTTTTAAAGCAAGTAAAACCTCTACAAAT |
| | | GTGGTACGGCTTGATTATGATCATGACTAAAGAATGTGGTTAGAGACAGT |
| | | CTGAGGAAATGTTTTCTGACTTTGTTTTGGTTTCCAACCAGAGCATCGTG |
| | | AGTGAGCTGGCGGGGCTTTTGTCAGCGATGGAGTACGTGCAGAAAACTC |
| | | TCACGGACGAGGAGCTGGCTGACTGGAAGAGGCGGCAACAGATTGCCT |
| | | GCATTGGAGGCCCGCCCAACATCTGCCTAGATCGGCTAGAAAACTGGTA |
| | | AAGGATGAAAGAAGCTTTTCCTTTCTTTCTCGAAAGCTAGATTGAATTCTG |
| | | ATCTTAACTGCAGGCCCACAGAATTGGTACTATATCTCCAACGTGGGGAC |
| | | TTTTCCATATTCAAATTTAGCCCAAGAATTAAAGTTTTTACTTTATTTCGGC |
| | | CAGGCGCTGTGGCTCACACCTGTAATCCCAGCACTTTGGGAGA |
| 45 | STAT3 with | GTCAAGGAGTATTCCCTCAGGTCAAGGAGTTTTTTCTTCCTTCGCAGACA |
| | homologies | TGCAAGATCTGAATGGAAACAACCAGTCAGTGACCAGGCAGAAGATGCA |
| | template | GCAGCTGGAACAGATGCTCACTGCGCTGGACCAGATGCGGAGAGTAAG |
| | sequence | GGCATAGGTCGGACCACTTCCCCCATGTGTCTCGCTCACTTGCGGGATT |
| | 1 penta | TCAGCGTCTTGTGGCAGAACTTGCTTGGTTTCTAAGAAGTTCCTGCTCTG |
| | (@ i7) | GAGTTGATACAAGCTAGGCCCTTTTGCTAATCATGTTCATACTGTCCCTTT |
| | pCLS37604 | TTTTTCCTCAGAGCATCGTGAGTGAGCTGGCTGGGTTGCTTTCAGCTATG |
| | | GAATATGTGCAAAAGACGTTGACAGACGAGGAACTGGCCGACTGGAAGC |
| | | GGAGGCAACAAATTGCTTGCATTGGAGGACCACCCAATATATGCCTGGA |
| | | TAGATTGGAGAACTGGATAACGTCATTAGCAGAAAGCCAACTGCAAACTA |
| | | GACAGCAAATAAAGAAGCTGGAAGAACTCCAGCAAAAAGTTTCCTATAAG |
| | | GGTGACCCTATAGTGCAGCACAGGCCCATGTTGGAGGAACGCATTGTGG |
| | | AGTTGTTTAGAAATCTGATGAAAAGTGCCTTTGTGGTGGAGCGACAACCG |
| | | TGCATGCCTATGCACCCTGACCGGCCCTTGGTGATAAAGACCGGAGTAC |
| | | AATTCACTACTAAAGTGAGGCTGCTCGTAAAGTTCCCTGAACTCAATTAC |
| | | CAGTTGAAGATAAAAGTGTGCATCGATAAAGACTCTGGGGACGTTGCAG |
| | | CTCTCAGAGGATCCCGGAAGTTCAACATCTTGGGCACCAACACAAAAGT |
| | | GATGAACATGGAAGAATCCAACAACGGCAGCCTCTCTGCCGAATTCAAA |
| | | CACTTGACCCTGAGGGAGCAGAGATGTGGGAATGGGGGCCGAGCCAAT |
| | | TGTGATGCTTCCCTGATTGTGACTGAGGAGCTGCACCTGATCACCTTTGA |
| | | GACCGAGGTGTATCACCAAGGCCTCAAGATTGACCTAGAGACCCACTCC |
| | | TTGCCAGTTGTGGTGATCTCCAACATCTGTCAGATGCCAAATGCCTGGGC |
| | | GTCCATCCTGTGGTACAACATGCTGACCAACAATCCCAAGAATGTAAACT |
| | | TTTTTACCAAGCCCCCAATTGGAACCTGGGATCAAGTGGCCGAGGTCCT |
| | | GAGCTGGCAGTTCTCCTCCACCACCAAGCGAGGACTGAGCATCGAACAA |
| | | TTGACGACCCTGGCAGAGAAACTCTTGGGACCTGGTGTGAATTATTCAG |
| | | GGTGTCAGATCACATGGGCTAAATTTTGCAAAGAAAACATGGCTGGCAAG |
| | | GGCTTCTCCTTCTGGGTCTGGCTGGACAATATCATTGACCTTGTGAAAAA |
| | | GTACATCCTGGCCCTTTGGAACGAAGGATACATCATGGGCTTTATCAGTA |
| | | AGGAGCGGGAGAGAGCGATCCTGTCAACTAAACCTCCAGGCACCTTCCT |
| | | GCTAAGATTCAGTGAAAGCAGCAAAGAAGGAGGCGTCACTTTCACTTGG |
| | | GTGGAGAAGGACATCAGCGGAAAGACGCAAATTCAGAGCGTGGAACCAT |
| | | ACACAAAGCAGCAGCTGAACAACATGTCATTTGCTGAAATCATCATGGGC |
| | | TATAAGATCATGGATGCTACCAATATCCTGGTGTCTCCACTGGTCTATCT |
| | | CTATCCGGACATTCCCAAAGAGGAGGCGTTCGGAAAGTACTGTCGCCCG |
| | | GAGTCACAGGAGCATCCTGAAGCTGACCCAGGCAGCGCTGCCCCATAC |
| | | CTGAAGACCAAGTTTATCTGTGTGACACCGTAAGTGGCTTCCTTTCCCCG |
| | | TTTTGCCTTCATTTCTAATATCCTCAGTTATCCCTGGGAATGGGACACTGG |
| | | GTGAGAGTTAATCTGCCAAAGGTTGGAAGCCCCTGGGCTATGTTTAGTAC |
| | | TCAAAGTGACCTTGTGTGTTTAAAAAGCTTGAGCTTTTATTTTTCTGTTGG |
| | | AGACCAGAGTTTGATGGCTTGTGTGTGTGTGTTTTGTTCTTTTTTTTTTTT |
| | | CCATTGTGTCTTGTCAACCCCCCGTTTCCCCTCCTGCTGCCCCCCATTTC |
| | | CTACAGAACGACCTGCAGCAATACCATTGACCTGCCGATGTCCCCCCGC |
| | | ACTTTAGATTCATTGATGCAGTTTGGAAATAATGGTGAAGGTGCTGAACC |
| | | CTCAGCAGGAGGACAGTTTGAGAGCCTTACGTTTGATATGGAGTTGACCT |
| | | CGGAGTGCGCTACCTCCCCCATGTGAGCAGATCCAGACATGATAAGATA |
| | | CATTGATGAGTTTGGACAAACCACAACTAGAATGCAGTGAAAAAAATGCT |
| | | TTATTTGTGAAATTTGTGATGCTATTGCTTTATTTGTAACCATTATAAGCTG |
| | | CAATAAACAAGTTAACAACAACAATTGCATTCATTTTATGTTTCAGGTTCA |
| | | GGGGGAGGTGTGGGAGGTTTTTTAAAGCAAGTAAAACCTCTACAAATGT |
| | | GGTACGGCTTGATTATGATCATGACTAAAGAATGTGGTTAGAGACAGTCT |
| | | GAGGAAATGTTTTCTGACTTTGTTTTGGTTTCCAACCAGAGCATCGTGAG |
| | | TGAGCTGGCGGGGCTTTTGTCAGCGATGGAGTACGTGCAGAAAACTCTC |
| | | ACGGACGAGGAGCTGGCTGACTGGAAGAGGCGGCAACAGATTGCCTGC |
| | | ATTGGAGGCCCGCCCAACATCTGCCTAGATCGGCTAGAAAACTGGTAAA |
| | | GGATGAAAGAAGCTTTTCCTTTCTTTCTCGAAAGCTAGATTGAATTCTGAT |
| | | CTTAACTGCAGGCCCACAGAATTGGTACTATATCTCCAACGTGGGGACTT |
| | | TTCCATATTCAAATTTAGCCCAAGAATTAAAGTTTTTACTTTATTTCGGCCA |
| | | GGCGCTGTGGCTCACACCTGTAATCCCAGCACTTTGGGAGA |
| 47 | STAT 3 with | GTCTCCATGTCTTCAGTATTTCCTTCCCCTTCTCCATCTCACCTGTATACA |
| | homologies | TTCACTTTGGTAATTAGCATCTTTCTTAATTTATTGGCAGGATAACGTCATT |
| | template | AGCAGAATCTCAACTTCAGACCCGTCAACAAATTAAGAAACTGGAGGAGT |
| | sequence | TGCAGCAAAAAGTTTCCTACAAAGGGGACCCCATTGTACAGCACCGGCC |
| | (@i9) | GATGCTGGAGGAGAGAATCGTGGAGCTGTTTAGAAACTTAATGAAAAGG |
| | pCLS35054 | TAATTTAGCATCCTTGTCCCTTTCCCTCATCTAAAAAATACCTAAAGACTC |
| | | ACGTGGTAGAGTGAGAGGCGGGCTGACTTCTGGTCATGGCCGTGGCGT |
| | | GATACAAGCTAGGCCCTTTTGCTAATCATGTTCATACTGTCCCTTTTTTTT |
| | | CCACAGTGCCTTTGTGGTGGAGCGACAACCGTGCATGCCTATGCACCCT |
| | | GACCGGCCCTTGGTGATAAAGACCGGAGTACAATTCACTACTAAAGTGA |
| | | GGCTGCTCGTAAAGTTCCCTGAACTCAATTACCAGTTGAAGATAAAAGTG |
| | | TGCATCGATAAGGACTCCGGCGATGTTGCCGCGCTCCGGGGAAGTCGA |
| | | AAGTTCAACATCTTGGGCACCAATACAAAGGTGATGAACATGGAGGAATC |
| | | AAACAACGGGTCCCTTTCCGCGGAGTTCAAACATTTGACATTGCGCGAG |
| | | CAGAGATGTGGGAACGGGGGAAGAGCAAATTGTGACGCTTCTCTGATCG |
| | | TAACTGAGGAGCTGCATCTCATAACATTCGAGACTGAGGTCTACCATCAG |
| | | GGATTGAAGATCGACCTCGAAACTCATTCACTGCCTGTAGTTGTTATTAG |
| | | TAATATTTGCCAGATGCCGAATGCGTGGGCTAGCATACTTTGGTATAACA |
| | | TGCTTACGAATAATCCAAAGAACGTTAATTTTTTCACTAAACCTCCAATCG |
| | | GAACTTGGGACCAGGTCGCAGAAGTGCTGAGCTGGCAATTTTCCTCCAC |
| | | CACCAAACGCGGTCTTAGCATCGAACAATTGACGACCCTCGCAGAAAAG |
| | | CTTTTGGGTCCTGGCGTTAATTACTCAGGGTGTCAGATAACTTGGGCAAA |
| | | GTTCTGCAAAGAAAACATGGCAGGTAAGGGCTTTTCATTTTGGGTCTGGC |
| | | TGGATAACATTATAGATTTGGTGAAAAAGTACATTCTTGCCCTGTGGAAC |
| | | GAAGGCTACATCATGGGTTTCATTAGCAAGGAGCGCGAGAGAGCGATCC |
| | | TGTCAACTAAACCCCCTGGAACGTTTCTCCTTAGATTTTCCGAAAGCAGC |
| | | AAGGAAGGTGGCGTGACTTTCACGTGGGTTGAAAAGGACATCAGCGGAA |
| | | AGACGCAAATTCAGAGCGTTGAACCTTACACCAAGCAGCAACTTAACAAC |
| | | ATGAGCTTCGCCGAGATAATAATGGGTTACAAAATAATGGACGCTACTAA |
| | | TATCCTCGTCTCACCCCTCGTGTACTTGTATCCAGACATTCCCAAAGAGG |
| | | AGGCGTTCGGGAAGTACTGTCGCCCGGAGTCACAAGAACATCCTGAAGC |
| | | TGACCCAGGAAGTGCAGCTCCGTACCTTAAGACCAAGTTTATCTGTGTGA |
| | | CACCGTAAGTGGCTTCCTTTCCCCGTTTTGCCTTCATTTCTAATATCCTCA |
| | | GTTATCCCTGGGAATGGGACACTGGGTGAGAGTTAATCTGCCAAAGGTT |
| | | GGAAGCCCCTGGGCTATGTTTAGTACTCAAAGTGACCTTGTGTGTTTAAA |
| | | AAGCTTGAGCTTTTATTTTTCTGTTGGAAGACCAGAGTTTGATGGCTTGTG |
| | | TGTGTGTGTTTTGTTCTTTTTTTTTTTTCCATTGTGTCTTGTCAACCCCCCG |
| | | TTTCCCCTCCTGCTGCCCCCCATTTCCTACAGAACGACCTGCAGCAATAC |
| | | CATTGACCTGCCGATGTCCCCCCGCACTTTAGATTCATTGATGCAGTTTG |
| | | GAAATAATGGTGAAGGTGCTGAACCATCCGCTGGTGGACAGTTTGAGAG |
| | | CCTTACGTTTGATATGGAGTTGACAAGCGAGTGTGCTACATCACCTATGT |
| | | GAGCAGATCCAGACATGATAAGATACATTGATGAGTTTGGACAAACCACA |
| | | ACTAGAATGCAGTGAAAAAAATGCTTTATTTGTGAAATTTGTGATGCTATT |
| | | GCTTTATTTGTAACCATTATAAGCTGCAATAAACAAGTTAACAACAACAAT |
| | | TGCATTCATTTTATGTTTCAGGTTCAGGGGGAGGTGTGGGAGGTTTTTTA |
| | | AAGCAAGTAAAACCTCTACAAATGTGGTACGGCTTGATTATGATCACGTG |
| | | AGCCCATCTTCTCTTTCCTCAGTGCCTTTGTGGTGGAGCGGCAGCCCTG |
| | | CATGCCCATGCATCCTGACCGGCCCCTCGTCATCAAGACCGGCGTCCAG |
| | | TTCACTACTAAAGTCAGGTAGGCCATGCCACTTCCATTTCCAGTAGAGAT |
| | | TTTACTGAGGGACACTGTTAGGGTGAGGGTAGAGTTGGTGGCCAGGGTC |
| | | ATTCTTTCCAGGTGTGGTGTCACAGGCAGTACACTGTTGCGGGGTTGAA |
| | | ATTTGTTGCCATACTATCTGCTTGCTCTCTGATTCTGATGTCAAAAGCAAA |
| | | AGAGCAGTCATCTTTTTGAAGGTACCTGGGCATATTCCTATGATTGTAGA |
| | | CCTGGAGTCTCAGGCCACAGCTTCTCCTTCTGCCCAAGGGACAAAATAAT |
| | | GTCATCTATTTTCTGTTCTTTGAGGCTACTCTTCCCTGTGGATTTTAAGGG |
| | | AAAGAGTAAGGCTTAGTGATGGGGAAGCTGAGAGGCCCCAGGGCAGGT |

### REFERENCES CITED IN THE APPLICATION

[1] Grimbacher B, Holland S M, Gallin J I, et al. Hyper-IgE syndrome with recurrent infections—an autosomal dominant multisystem disorder. N Engl J Med. 340(9), 692-702 (1999)
[2] Holland S M, DeLeo F R, Elloumi H Z, et al. STAT3 mutations in the hyper-IgE syndrome. N Engl J Med. 357(16), 1608-1619 (2007)
[3] Minegishi Y, Saito M, Tsuchiya S, et al. Dominant-negative mutations in the DNA-binding domain of STAT3 cause hyper-IgE syndrome. Nature. 448(7157): 1058-1062 (2007)
[4] Jiao H, Toth B, Erdos M, et al. Novel and recurrent STAT3 mutations in hyper-IgE syndrome patients from different ethnic groups. Mol Immunol. 46(1), 202-206 (2008).
[5] Renner E D, Rylaarsdam S, Anover-Sombke S, et al. Novel signal transducer and activator of transcription 3 (STAT3) mutations, reduced T(H)17 cell numbers, and variably defective STAT3 phosphorylation in hyper-IgE syndrome. J Allergy Clin Immunol. 122(1), 181-187 (2008)
[6] Takeda K, Noguchi K, Shi W, et al. Targeted disruption of the mouse Stat3 gene leads to early embryonic lethality. Proc Natl Acad Sci USA. 94(8), 3801-3804 (1997)
[7] de Beaucoudrey L, Puel A, Filipe-Santos O, et al. Mutations in STAT3 and IL12RB1 impair the development of human IL-17-producing T cells. J Exp Med. 205(7), 1543-1550 (2008)
[8] Harris T J, Grosso J F, Yen H R, et al. Cutting edge: An in vivo requirement for STAT3 signaling in TH17 development and TH17-dependent autoimmunity. J Immunol. 179(7), 4313-4317 (2007)
[9] Ma C S, Chew G Y, Simpson N, et al. Deficiency of Th17 cells in hyper IgE syndrome due to mutations in STAT3. J Exp Med. 205(7), 1551-1557 (2008)
[10] Milner J D, Brenchley J M, Laurence A, et al. Impaired T(H) 17 cell differentiation in subjects with autosomal dominant hyper-IgE syndrome. Nature 452(7188), 773-776 (2008)
[11] Aigner P, Just V, Stoiber D. STAT3 isoforms: Alternative fates in cancer? Cytokine (2018)
[12] Lorenzini T, Dotta L, Giacomelli M, Vairo D, Badolato R. STAT mutations as program switchers: turning primary immunodeficiencies into autoimmune diseases. J Leukoc Biol. 101(1), 29-38 (2017)
[13] Cornu TI, Mussolino C, Cathomen T. Refining strategies to translate genome editing to the clinic. Nat Med. 23(4), 415-423 (2017)
[14] Dreyer A-K, Hoffmann D, Lachmann N, et al. TALEN-mediated functional correction of X-linked chronic granulomatous disease in patient-derived induced pluripotent stem cells. Biomaterials 69, 191-200 (2015)
[15] Owens, B. et al. Transcription activator like effector (TALE)-directed piggyBac transposition in human cells. Nucleic Acids Res. 41(19), 9197-207 (2013)
[16] Voigt, K. et al. Retargeting Sleeping Beauty Transposon Insertions by Engineered Zinc Finger DNA-binding Domains. Molecular Therapy 20(10), 1852-1862 (2012)
[17] Bhatt S. and Chalmers R. Targeted DNA transposition in vitro using a dCas9-transposase fusion protein. Nucleic Acids Res. 47(15), 8126-8135 (2019)
[18] Sather, B. D. et al. Efficient modification of CCR5 in primary human hematopoietic cells using a megaTAL nuclease and AAV donor template. Science translational medicine, 7(307), 307ra156 (2015)
[19] Komor et al. Programmable editing of a target base in genomic DNA without double-stranded DNA cleavage. Nature 533(7603), 420-424 (2016)
[20] Urnov F., et al. Highly efficient endogenous human gene correction using designed zinc-finger nucleases. Nature 435, 646-651 (2005)
[21] Mussolino et al. A novel TALE nuclease scaffold enables high genome editing activity in combination with low toxicity. Nucl. Acids Res. 39(21), 9283-9293 (2011)
[22] Boissel et al. MegaTALs: a rare-cleaving nuclease architecture for therapeutic genome engineering. Nucleic Acids Research 42 (4), 2591-2601 (2013)
[23] Mussolino et al. TALEN® facilitate targeted genome editing in human cells with high specificity and low cytotoxicity. Nucl. Acids Res. 42(10), 6762-6773 (2014)
[24] Doudna, J. et al. The new frontier of genome engineering with CRISPR-Cas9. Science 346 (6213), 1077 (2014)
[25] Zetsche, B. et al. Cpf1 Is a Single RNA-Guided Endonuclease of a Class 2 CRISPR-Cas System. Cell 163(3), 759-771 (2015)

1-55. (canceled)

56. A population of engineered hematopoietic stem cells (HSCs) or T-cells originating from a patient suffering from Hyper-lgE Syndrome (HIES), comprising:
an exogenous polynucleotide comprising a partial or complete polynucleotide sequence encoding a functional STAT3 protein, wherein the exogenous polynucleotide is integrated in an endogenous STAT3 gene comprising at least one mutation causing HIES;
wherein the exogenous polynucleotide comprises an intronic sequence comprising a splice site that is:
(1) located upstream of the partial or complete polynucleotide sequence encoding the functional STAT3 protein; or
(2) located upstream of Exon 23 (SEQ ID NO:17) of STAT3 and enables an alternative splicing to Exon 23.

57. The population of engineered HSCs or T-cells according to claim 56, wherein the splice site is an artificial splice and comprises SEQ ID NO: 28 or SEQ ID NO: 29.

58. The population of engineered HSCs or T-cells according to claim 56, wherein the intronic sequence comprises at least 80% nucleic acid sequence identity with Intron 22 of STAT3 (SEQ ID NO: 27).

59. The population of engineered HSCs or T-cells according to claim 56, wherein the exogenous polynucleotide integrated into the endogenous STAT3 gene further comprises any one of Exons 8 to 24 of STAT3 (SEQ ID NOs: 2 to 18, respectively).

60. The population of engineered HSCs or T-cells according to claim 56, wherein the exogenous polynucleotide sequence is inserted into an intron of the endogenous STAT3 gene selected from Intron 7 (SEQ ID NO; 32), Intron 8 (SEQ ID NO: 33), or Intron 9 (SEQ ID NO: 34).

61. The population of engineered HSCs or T-cells according to claim 56, wherein the partial or complete polynucleotide sequence encoding the functional STAT3 protein is codon optimized.

62. The population of engineered HSCs or T-cells according to claim 56, wherein the exogenous polynucleotide sequence facilitates expression of STAT3alpha and STAT3beta in the engineered HSCs or T-cells.

63. The population of engineered HSCs or T-cells according to claim 56, wherein the engineered cells express STAT3alpha and STAT3beta isoforms in a ratio from about 3:1 to about 7:1.

64. The population of engineered HSCs or T-cells according to claim 56, wherein the engineered cells express STAT3alpha and STAT3beta isoforms in a ratio from about 4:1 to about 6:1.

65. A pharmaceutical composition comprising the population of cells of claim 56, and a pharmaceutically acceptable excipient and/or carrier.

66. A method of treating a subject having Hyper-IgE Syndrome (HIES) comprising administering the pharmaceutical composition of claim 65 to the subject.

67. The method of claim 66, wherein the method comprises stem cell transplantation and/or bone marrow transplantation.

68. A polynucleotide donor template comprising at least a partial or complete polynucleotide sequence encoding a functional STAT3 protein, wherein the donor template comprises at least one exon selected from Exons 8 to 24 of STAT3 (SEQ ID NOs: 2 to 18, respectively).

69. The polynucleotide donor template according to claim 68, further comprising at least Exon 23 of STAT3 (SEQ ID NO: 17), and optionally further comprising Exon 24 of STAT3 (SEQ ID NO: 18).

70. The polynucleotide donor template according to claim 69, comprising:
(1) Intron 22 of STAT3 (SEQ ID NO: 27), located upstream of Exon 23 and enables an alternative splicing to Exon 23; or
(2) an artificial splice site upstream of the partial or complete polynucleotide sequence encoding the functional STAT3 protein, wherein the artificial splice site comprises SEQ ID NO: 28 or SEQ ID NO: 29.

71. The polynucleotide donor template according to claim 68, wherein the partial or complete polynucleotide sequence encoding the functional STAT3 protein is codon optimized.

72. A method for engineering a population of T-cells or HSCs comprising the steps of:
introducing the polynucleotide donor template of claim 68 into a population of T-cells or HSCs originating from a patient suffering from HIES;
introducing into the T-cells or HSCs a sequence-specific reagent to cleave an intron sequence in the endogenous STAT3 gene, wherein the intron sequence comprises Intron 7 (SEQ ID NO; 32), Intron 8 (SEQ ID NO: 33), or Intron 9 (SEQ ID NO: 34), and inserting at this locus the polynucleotide donor template of claim 68 by homologous recombination or NHEJ; and
optionally, culturing the cells for expression of STAT3alpha and STAT3beta isoforms.
